(19) United States
(12) Patent Application Publication
(10) Pub. No.: US 2024/0360412 A1
(43) Pub. Date: Oct. 31, 2024

(54) CELL CULTURE MEDIUM FOR NATURAL KILLER (NK) CELLS

(71) Applicant: ANYADI, LLC, Wilmington, DE (US)

(72) Inventors: Rachit OHRI, Ellicott City, MD (US); Donna SONNTAG, Merrimack, NH (US); Jason CAHOON, Farmington, CT (US); Graeme LAMBERT, Canaan, NH (US)

(21) Appl. No.: 18/294,221

(22) PCT Filed: Aug. 4, 2022
(86) PCT No.: PCT/US2022/039376

§ 371 Date: Feb. 1, 2024

Related U.S. Application Data
(60) Provisional application No. 63/230,189, filed on Aug. 6, 2021.

(51) Int. Cl. C12N 5/0783

(52) U.S. Cl. CPC C12N 5/0646; C12N 2500/24; C12N 2500/32; C12N 2500/38; C12N 2500/76; C12N 2501/105; C12N 2501/33; C12N 2501/599

(57) ABSTRACT

The invention of the current disclosure provides cell culture medium formulations useful for the culture of Natural Killer (NK) immune cells. Also included are methods of culture, cryopreservation, and transduction of NK cells comprising said culture medium formulations.

### CROSS REFERENCE TO RELATED APPLICATION

The present application is entitled to priority under 35 U.S.C. § 119(e) to U.S. Provisional Patent Application No. 63/230,189, filed Aug. 6, 2021, which is hereby incorporated by reference in its entirety herein.

### BACKGROUND OF THE INVENTION

The ex vivo and/or in vitro maintenance, research, or expansion of cellular therapies requires liquid media that are specially formulated for each category or type of cell—as a relevant source of energy and compounds for the maintenance of cellular health, function and phenotype of the cells, as well as for regulation of their cell cycle.

Existing media for the cell culture of NK cells is limited in supporting the activation and cytotoxic performance of the NK cells cultured in them for maintenance and/or proliferation, especially in the absence of supplemental cytokines and/or feeder cells. Specifically, the dependence of these media on specific ingredients (e.g. cytokines like Interleukin-15, IL-15 and others) results in the “exhaustion” of cultured NK cells, characterized by the decreased production of effector molecules (e.g. Interferon-gamma, IFN-γ), and impaired cytotoxic activity against target cells such as cancer cells. Moreover, these media often require significant amounts of animal serum (e.g. fetal bovine serum, human serum etc) typically in the range of 10-20% volume/volume (v/v), which then becomes the source of increased batch-to-batch variability, as well as increased cost and contamination risk of NK cellular immunotherapies. Additionally, the co-culturing of Antigen Presenting Cells (APCs, i.e. feeder cells) to achieve adequate expansion of NK cells introduces undesirable cost, complexity, and quality challenges for NK cellular therapies. Cryopreservation of NK cells too is challenging, due to decrease in viability, post-thaw cytotoxic activity, and other functions of NK cells. NK cell metabolism, transduction, subpopulation/subset content and differentiation are also sub-optimal.

Therefore, there is a need in the art for NK cell culture media formulations and techniques that address the challenges and problems with current in vitro and ex vivo culture systems for the generation of more efficacious NK cellular immunotherapies. The current invention addresses this need.

### SUMMARY OF THE INVENTION

The present invention relates to media formulations useful for the ex vivo culturing or genetic modification or preservation of Natural Killer (NK) immune cells. In particular, aspects of the present invention relate to research & development (R&D) on NK cellular immunotherapies, as well as the biomanufacturing (e.g. bioreactor expansion) and clinical use of cellular immunotherapies using Natural Killer (NK) cells.

As such, in one aspect, the invention includes a two-part media formulation for culturing Natural Killer (NK) cells comprising:
a. a first media formulation comprising at least six of following components:
   i. Glycine at a concentration between 3.50E−03 mM and 3.50E+02 mM;
   ii. L-Alanine at a concentration between 1.10E−02 mM and 1.10E+03 mM;
   iii. L-Asparagine at a concentration between 1.50E−03 mM and 1.50E+02 mM;
   iv. L-Aspartic acid at a concentration between 1.50E−03 mM and 1.50E+02 mM;
   v. L-Glutamic Acid at a concentration between 1.50E−03 mM and 1.50E+02 mM;
   vi. L-Proline at a concentration between 2.50E−03 mM and 2.50E+02 mM;
   vii. L-Serine at a concentration between 3.50E−03 mM and 3.50E+02 mM;
   viii. L-Arginine HCl at a concentration between 1.30E−02 mM and 1.30E+03 mM;
   ix. L-Cystine at a concentration between 2.00E−03 mM and 2.00E+02 mM;
   x. L-Histidine HCl—H₂O at a concentration between 3.50E−03 mM and 3.50E+02 mM;
   xi. L-Isoleucine at a concentration between 8.20E−03 mM and 8.20E+02 mM;
   xii. L-Leucine at a concentration between 8.50E−03 mM and 8.50E+02 mM;
   xiii. L-Lysine hydrochloride at a concentration between 8.96E−03 mM and 8.96E+02 mM;
   xiv. L-Methionine at a concentration between 2.21E−03 mM and 2.21E+02 mM;
   xv. L-Phenylalanine at a concentration between 4.20E−03 mM and 4.20E+02 mM;
   xvi. L-Threonine at a concentration between 8.50E−03 mM and 8.50E+02 mM;
   xvii. L-Tryptophan at a concentration between 9.00E−04 mM and 9.00E+01 mM;
   xviii. L-Tyrosine at a concentration between 4.09E−03 mM and 4.09E+02 mM;
   xix. L-Valine at a concentration between 8.50E−03 mM and 8.50E+02 mM;
   xx. Sodium Pyruvate at a concentration between 1.00E−02 mM and 1.00E+03 mM;
   xxi. Arachidonic acid at a concentration between 3.28E−07 mM and 3.28E−02 mM;
   xxii. Linoleic acid at a concentration between 3.78E−06 mM and 3.78E−01 mM;
   xxiii. Linolenic acid at a concentration between 1.80E−06 mM and 1.80E−01 mM;
   xxiv. Myristic acid at a concentration between 2.19E−06 mM and 2.19E−01 mM;
   xxv. Oleic acid at a concentration between 1.77E−06 mM and 1.77E−01 mM;
   xxvi. Palmitic acid at a concentration between 1.95E−06 mM and 1.95E−01 mM;
   xxvii. Stearic acid at a concentration between 1.76E−06 mM and 1.76E−01 mM;
   xxviii. Cholesterol at a concentration between 2.84E−05 mM and 2.84E+00 mM;
   xxix. Tween-80 at a concentration between 8.40E−05 mM and 8.40E+00 mM;
   xxx. Tocopherol acetate at a concentration between 7.40E−06 mM and 7.40E−01 mM;
   xxxi. Pluronic F-68 at a concentration between 5.95E−04 mM and 5.95E+01 mM;
   xxxii. Insulin at a concentration between 1.72E−05 mM and 1.72E+00 mM;
   xxxiii. Transferrin at a concentration between 6.88E−07 mM and 6.88E−02 mM;
   xxxiv. Sodium Selenite at a concentration between 3.87E−07 mM and 3.87E−02 mM;
   xxxv. myo-Inositol at a concentration between 2.00E−03 mM and 2.00E+02 mM;
   xxxvi. Glucose at a concentration between 2.22E−01 mM and 2.22E+04 mM;
   xxxvii. Human Serum Albumin (HSA) at a concentration between 1.50E−05 mM and 1.50E+00 mM;
   xxxviii. Biotin at a concentration between 8.10E−06 mM and 8.10E−01 mM;
   xxxix. Folic Acid at a concentration between 2.40E−04 mM and 2.40E+01 mM;
   xL. Vitamin B12 at a concentration between 1.00E−02 mM and 1.00E+03 mM;
   xLi. Vitamin C at a concentration between 2.84E−03 mM and 2.84E+02 mM;
   xLii. Vitamin E at a concentration between 2.40E−03 mM and 2.40E+02 mM;
   xLiii. Zinc at a concentration between 1.50E−08 mM and 1.50E−03 mM;
   xLiv. L-Ornithine at a concentration between 5.00E−04 mM and 5.00E+01 mM;
   xLv. Creatine Anhydrous at a concentration between 1.00E−02 mM and 1.00E+03 mM;
   xLvi. PolyVinyl Alcohol (PVA) at a concentration between 1.16E−01 and 1.16E+04 mM;
   xLvii. Ethanolamine at a concentration between 5.00E−04 and 5.00E+01 mM;
   xLviii. 2-mercaptoethanol (2-ME) at a concentration between 1.00E−03 mM and 1.00E+02 mM;
   xLix. Androstenedione at a concentration between 1.00E−05 and 1.00E+00 mM;
   L. Dextran at a concentration between 1.00E+01 and 1.00E+06 mg/L;
   Li. L-Alanyl-L-Glutamine (Glutamax) at a concentration between 2.50E−02 mM and 2.50E+03 mM;
   Lii. a polyamine supplement at a concentration between 0.01× and 1000× of a 1000× stock (polyamine supplement being Sigma-Aldrich Catalog #P8483 or equivalent); and
   Liii. an antioxidant supplement at a concentration between 0.01× and 1000× of a 1000× (antioxidant supplement being Sigma-Aldrich Catalog #A1345 or equivalent); and
b. a second media formulation comprising the first media formulation and at least three of the following components:
   Liv. Nicotinamide at a concentration between 2.50E−02 mM and 2.50E+03 mM;
   Lv. IGF-1 (Insulin like Growth Factor 1) at a concentration between 2.60E−08 mM and 2.60E−03 mM;
   Lvi. 4-1BBL [4-1BB Ligand or ligand for Cluster of Differentiation 137 (CD137)] at a concentration between 4.20E−09 mM and 4.20E−04 mM;
   Lvii. Ashwagandha (botanical name:Withania Somnifera) plant extract at a concentration between 6.00E−01 mg/L and 6.00E+04 mg/L;
   Lviii. Kumquat (botanical name: Fortunella Japonica) plant extract at a concentration between 1.40E−22 mg/L and 1.40E−02 mg/L;
   Lix. Umckaloabo (botanical name: Pelargonium sidoides) plant extract at a concentration between 5.00E−01 mg/L and 5.00E+05 mg/L;
   Lx. Echinacea (botanical name: Echinacea Purpurea) plant extract at a concentration between 1.01E−01 mg/L and 1.00E+05 mg/L;
   Lxi. Red Ginseng (botanical name: Panax ginseng) plant extract at a concentration between 8.43E−02 mg/L and 8.43E+03 mg/L;
   Lxii. 2,3-Butanediol at a concentration between 9.01E−02 mg/L and 9.01E+03 mg/L; and
   Lxiii. Elderberry (botanical name: Sambucus nigra) plant extract at a concentration between 1.06E−01 mg/L and 1.06E+04 mg/L;
      wherein the NK cells are cultured in the first media formulation prior to culture in the second media formulation.

In another aspect, the invention includes a one-part media formulation for culturing Natural Killer (NK) cells comprising at least six of the following components:
i. Glycine at a concentration between 3.50E−03 mM and 3.50E+02 mM;
ii. L-Alanine at a concentration between 1.10E−02 mM and 1.10E+03 mM;
iii. L-Asparagine at a concentration between 1.50E−03 mM and 1.50E+02 mM;
iv. L-Aspartic acid at a concentration between 1.50E−03 mM and 1.50E+02 mM;
v. L-Glutamic Acid at a concentration between 1.50E−03 mM and 1.50E+02 mM;
vi. L-Proline at a concentration between 2.50E−03 mM and 2.50E+02 mM;
vii. L-Serine at a concentration between 3.50E−03 mM and 3.50E+02 mM;
viii. L-Arginine HCl at a concentration between 1.30E−02 mM and 1.30E+03 mM;
ix. L-Cystine at a concentration between 2.00E−03 mM and 2.00E+02 mM;
x. L-Histidine HCl—H₂O at a concentration between 3.50E−03 mM and 3.50E+02 mM;
xi. L-Isoleucine at a concentration between 8.20E−03 mM and 8.20E+02 mM;
xii. L-Leucine at a concentration between 8.50E−03 mM and 8.50E+02 mM;
xiii. L-Lysine hydrochloride at a concentration between 8.96E−03 mM and 8.96E+02 mM;
xiv. L-Methionine at a concentration between 2.21E−03 mM and 2.21E+02 mM;
xv. L-Phenylalanine at a concentration between 4.20E−03 mM and 4.20E+02 mM;
xvi. L-Threonine at a concentration between 8.50E−03 mM and 8.50E+02 mM;
xvii. L-Tryptophan at a concentration between 9.00E−04 mM and 9.00E+01 mM;
xviii. L-Tyrosine at a concentration between 4.09E−03 mM and 4.09E+02 mM;
xix. L-Valine at a concentration between 8.50E−03 mM and 8.50E+02 mM;
xx. Sodium Pyruvate at a concentration between 1.00E−02 mM and 1.00E+03 mM;
xxi. Arachidonic acid at a concentration between 3.28E−07 mM and 3.28E−02 mM;
xxii. Linoleic acid at a concentration between 3.78E−06 mM and 3.78E−01 mM;
xxiii. Linolenic acid at a concentration between 1.80E−06 mM and 1.80E−01 mM;
xxiv. Myristic acid at a concentration between 2.19E−06 mM and 2.19E−01 mM;
xxv. Oleic acid at a concentration between 1.77E−06 mM and 1.77E−01 mM;
xxvi. Palmitic acid at a concentration between 1.95E−06 mM and 1.95E−01 mM;
xxvii. Stearic acid at a concentration between 1.76E−06 mM and 1.76E−01 mM;
xxviii. Cholesterol at a concentration between 2.84E−05 mM and 2.84E+00 mM;
xxix. Tween-80 at a concentration between 8.40E−05 mM and 8.40E+00 mM;
xxx. Tocopherol acetate at a concentration between 7.40E−06 mM and 7.40E−01 mM;
xxxi. Pluronic F-68 at a concentration between 5.95E−04 mM and 5.95E+01 mM;
xxxii. Insulin at a concentration between 1.72E−05 mM and 1.72E+00 mM;
xxxiii. Transferrin at a concentration between 6.88E−07 mM and 6.88E−02 mM;
xxxiv. Sodium Selenite at a concentration between 3.87E−07 mM and 3.87E−02 mM;
xxxv. myo-Inositol at a concentration between 2.00E−03 mM and 2.00E+02 mM;
xxxvi. Glucose at a concentration between 2.22E−01 mM and 2.22E+04 mM;
xxxvii. Human Serum Albumin (HSA) at a concentration between 1.50E−05 mM and 1.50E+00 mM;
xxxviii. Biotin at a concentration between 8.10E−06 mM and 8.10E−01 mM;
xxxix. Folic Acid at a concentration between 2.40E−04 mM and 2.40E+01 mM;
xL. Vitamin B12 at a concentration between 1.00E−02 mM and 1.00E+03 mM;
xLi. Vitamin C at a concentration between 2.84E−03 mM and 2.84E+02 mM;
xLii. Vitamin E at a concentration between 2.40E−03 mM and 2.40E+02 mM;
xLiii. Zinc at a concentration between 1.50E−08 mM and 1.50E−03 mM;
xLiv. L-Ornithine at a concentration between 5.00E−04 mM and 5.00E+01 mM;
xLv. Creatine Anhydrous at a concentration between 1.00E−02 mM and 1.00E+03 mM;
xLvi. PolyVinyl Alcohol (PVA) at a concentration between 1.16E−01 and 1.16E+04 mM;
xLvii. Ethanolamine at a concentration between 5.00E−04 and 5.00E+01 mM;
xLviii. 2-mercaptoethanol (2-ME) at a concentration between 1.00E−03 mM and 1.00E+02 mM;
xLix. Androstenedione at a concentration between 1.00E−05 and 1.00E+00 mM;
L. Dextran at a concentration between 1.00E+01 and 1.00E+06 mg/L;
Li. L-Alanyl-L-Glutamine (Glutamax) at a concentration between 2.50E−02 mM and 2.50E+03 mM;
Lii. a polyamine supplement at a concentration between 0.01× and 1000× of a 1000× stock (polyamine supplement being Sigma-Aldrich Catalog #P8483 or equivalent); and
Liii. an antioxidant supplement at a concentration between 0.01× and 1000× of a 1000× (antioxidant supplement being Sigma-Aldrich Catalog #A1345 or equivalent).

In another aspect, the invention includes a one-part media formulation culturing Natural Killer (NK) cells comprising at least nine of the following components:
i. Glycine at a concentration between 3.50E−03 mM and 3.50E+02 mM;
ii. L-Alanine at a concentration between 1.10E−02 mM and 1.10E+03 mM;
iii. L-Asparagine at a concentration between 1.50E−03 mM and 1.50E+02 mM;
iv. L-Aspartic acid at a concentration between 1.50E−03 mM and 1.50E+02 mM;
v. L-Glutamic Acid at a concentration between 1.50E−03 mM and 1.50E+02 mM;
vi. L-Proline at a concentration between 2.50E−03 mM and 2.50E+02 mM;
vii. L-Serine at a concentration between 3.50E−03 mM and 3.50E+02 mM;
viii. L-Arginine HCl at a concentration between 1.30E−02 mM and 1.30E+03 mM;
ix. L-Cystine at a concentration between 2.00E−03 mM and 2.00E+02 mM;
x. L-Histidine HCl—H₂O at a concentration between 3.50E−03 mM and 3.50E+02 mM;
xi. L-Isoleucine at a concentration between 8.20E−03 mM and 8.20E+02 mM;
xii. L-Leucine at a concentration between 8.50E−03 mM and 8.50E+02 mM;
xiii. L-Lysine hydrochloride at a concentration between 8.96E−03 mM and 8.96E+02 mM;
xiv. L-Methionine at a concentration between 2.21E−03 mM and 2.21E+02 mM;
xv. L-Phenylalanine at a concentration between 4.20E−03 mM and 4.20E+02 mM;
xvi. L-Threonine at a concentration between 8.50E−03 mM and 8.50E+02 mM;
xvii. L-Tryptophan at a concentration between 9.00E−04 mM and 9.00E+01 mM;
xviii. L-Tyrosine at a concentration between 4.09E−03 mM and 4.09E+02 mM;
xix. L-Valine at a concentration between 8.50E−03 mM and 8.50E+02 mM;
xx. Sodium Pyruvate at a concentration between 1.00E−02 mM and 1.00E+03 mM;
xxi. Arachidonic acid at a concentration between 3.28E−07 mM and 3.28E−02 mM;
xxii. Linoleic acid at a concentration between 3.78E−06 mM and 3.78E−01 mM;
xxiii. Linolenic acid at a concentration between 1.80E−06 mM and 1.80E−01 mM;
xxiv. Myristic acid at a concentration between 2.19E−06 mM and 2.19E−01 mM;
xxv. Oleic acid at a concentration between 1.77E−06 mM and 1.77E−01 mM;
xxvi. Palmitic acid at a concentration between 1.95E−06 mM and 1.95E−01 mM;
xxvii. Stearic acid at a concentration between 1.76E−06 mM and 1.76E−01 mM;
xxviii. Cholesterol at a concentration between 2.84E−05 mM and 2.84E+00 mM;
xxix. Tween-80 at a concentration between 8.40E−05 mM and 8.40E+00 mM;
xxx. Tocopherol acetate at a concentration between 7.40E−06 mM and 7.40E−01 mM;
xxxi. Pluronic F-68 at a concentration between 5.95E−04 mM and 5.95E+01 mM;
xxxii. Insulin at a concentration between 1.72E−05 mM and 1.72E+00 mM;
xxxiii. Transferrin at a concentration between 6.88E−07 mM and 6.88E−02 mM;
xxxiv. Sodium Selenite at a concentration between 3.87E−07 mM and 3.87E−02 mM;
xxxv. myo-Inositol at a concentration between 2.00E−03 mM and 2.00E+02 mM;
xxxvi. Glucose at a concentration between 2.22E−01 mM and 2.22E+04 mM;
xxxvii. Human Serum Albumin (HSA) at a concentration between 1.50E−05 mM and 1.50E+00 mM;
xxxviii. Biotin at a concentration between 8.10E−06 mM and 8.10E−01 mM;
xxxix. Folic Acid at a concentration between 2.40E−04 mM and 2.40E+01 mM;
xL. Vitamin B12 at a concentration between 1.00E−02 mM and 1.00E+03 mM;
xLi. Vitamin C at a concentration between 2.84E−03 mM and 2.84E+02 mM;
xLii. Vitamin E at a concentration between 2.40E−03 mM and 2.40E+02 mM;
xLiii. Zinc at a concentration between 1.50E−08 mM and 1.50E−03 mM;
xLiv. L-Ornithine at a concentration between 5.00E−04 mM and 5.00E+01 mM;
xLv. Creatine Anhydrous at a concentration between 1.00E−02 mM and 1.00E+03 mM;
xLvi. PolyVinyl Alcohol (PVA) at a concentration between 1.16E−01 and 1.16E+04 mM;
xLvii. Ethanolamine at a concentration between 5.00E−04 and 5.00E+01 mM;
xLviii. 2-mercaptoethanol (2-ME) at a concentration between 1.00E−03 mM and 1.00E+02 mM;
xLix. Androstenedione at a concentration between 1.00E−05 and 1.00E+00 mM;
L. Dextran at a concentration between 1.00E+01 and 1.00E+06 mg/L;
Li. L-Alanyl-L-Glutamine (Glutamax) at a concentration between 2.50E−02 mM and 2.50E+03 mM;
Lii. a polyamine supplement at a concentration between 0.01× and 1000× of a 1000× stock (polyamine supplement being Sigma-Aldrich Catalog #P8483 or equivalent);
Liii. an antioxidant supplement at a concentration between 0.01× and 1000× of a 1000× (antioxidant supplement being Sigma-Aldrich Catalog #A1345 or equivalent);
Liv. Nicotinamide at a concentration between 2.50E−02 mM and 2.50E+03 mM;
Lv. IGF-1 (Insulin like Growth Factor 1) at a concentration between 2.60E−08 mM and 2.60E−03 mM;
Lvi. 4-1BBL [4-1BB Ligand or ligand for Cluster of Differentiation 137 (CD137)] at a concentration between 4.20E−09 mM and 4.20E−04 mM;
Lvii. Ashwagandha (botanical name: Withania Somnifera) plant extract at a concentration between 6.00E−01 mg/L and 6.00E+04 mg/L;
Lviii. Kumquat (botanical name: Fortunella Japonica) plant extract at a concentration between 1.40E−22 mg/L and 1.40E−02 mg/L;
Lix. Umckaloabo (botanical name: Pelargonium sidoides) plant extract at a concentration between 5.00E−01 mg/L and 5.00E+05 mg/L;
Lx. Echinacea (botanical name: Echinacea Purpurea) plant extract at a concentration between 1.01E−01 mg/L and 1.00E+05 mg/L;
Lxi. Red Ginseng (botanical name: Panax ginseng) plant extract at a concentration between 8.43E−02 mg/L and 8.43E+03 mg/L;
Lxii. 2,3-Butanediol at a concentration between 9.01E−02 mg/L and 9.01E+03 mg/L; and
Lxiii. Elderberry (botanical name: Sambucus nigra) plant extract at a concentration between 1.06E−01 mg/L and 1.06E+04 mg/L

In certain embodiments, the formulation is added to a base cell culture media as a supplement.

In another aspect, the invention includes a method for culturing Natural Killer (NK) cells or precursor cells thereof comprising culturing the NK cells or precursor cells thereof in a cell culture media formulation comprising:
a. a first media formulation comprising at least six of the following components:
   i. Glycine at a concentration between 3.50E−03 mM and 3.50E+02 mM;
   ii. L-Alanine at a concentration between 1.10E−02 mM and 1.10E+03 mM;
   iii. L-Asparagine at a concentration between 1.50E−03 mM and 1.50E+02 mM;
   iv. L-Aspartic acid at a concentration between 1.50E−03 mM and 1.50E+02 mM;
   v. L-Glutamic Acid at a concentration between 1.50E−03 mM and 1.50E+02 mM;
   vi. L-Proline at a concentration between 2.50E−03 mM and 2.50E+02 mM;
   vii. L-Serine at a concentration between 3.50E−03 mM and 3.50E+02 mM;
   viii. L-Arginine HCl at a concentration between 1.30E−02 mM and 1.30E+03 mM;
   ix. L-Cystine at a concentration between 2.00E−03 mM and 2.00E+02 mM;
   x. L-Histidine HCl—H₂O at a concentration between 3.50E−03 mM and 3.50E+02 mM;
   xi. L-Isoleucine at a concentration between 8.20E−03 mM and 8.20E+02 mM;
   xii. L-Leucine at a concentration between 8.50E−03 mM and 8.50E+02 mM;
   xiii. L-Lysine hydrochloride at a concentration between 8.96E−03 mM and 8.96E+02 mM;
   xiv. L-Methionine at a concentration between 2.21E−03 mM and 2.21E+02 mM;
   xv. L-Phenylalanine at a concentration between 4.20E−03 mM and 4.20E+02 mM;
   xvi. L-Threonine at a concentration between 8.50E−03 mM and 8.50E+02 mM;
   xvii. L-Tryptophan at a concentration between 9.00E−04 mM and 9.00E+01 mM;
   xviii. L-Tyrosine at a concentration between 4.09E−03 mM and 4.09E+02 mM;
   xix. L-Valine at a concentration between 8.50E−03 mM and 8.50E+02 mM;
   xx. Sodium Pyruvate at a concentration between 1.00E−02 mM and 1.00E+03 mM;
   xxi. Arachidonic acid at a concentration between 3.28E−07 mM and 3.28E−02 mM;
   xxii. Linoleic acid at a concentration between 3.78E−06 mM and 3.78E−01 mM;
   xxiii. Linolenic acid at a concentration between 1.80E−06 mM and 1.80E−01 mM;
   xxiv. Myristic acid at a concentration between 2.19E−06 mM and 2.19E−01 mM;
   xxv. Oleic acid at a concentration between 1.77E−06 mM and 1.77E−01 mM;
   xxvi. Palmitic acid at a concentration between 1.95E−06 mM and 1.95E−01 mM;
   xxvii. Stearic acid at a concentration between 1.76E−06 mM and 1.76E−01 mM;
   xxviii. Cholesterol at a concentration between 2.84E−05 mM and 2.84E+00 mM;
   xxix. Tween-80 at a concentration between 8.40E−05 mM and 8.40E+00 mM;
   xxx. Tocopherol acetate at a concentration between 7.40E−06 mM and 7.40E−01 mM;
   xxxi. Pluronic F-68 at a concentration between 5.95E−04 mM and 5.95E+01 mM;
   xxxii. Insulin at a concentration between 1.72E−05 mM and 1.72E+00 mM;
   xxxiii. Transferrin at a concentration between 6.88E−07 mM and 6.88E−02 mM;
   xxxiv. Sodium Selenite at a concentration between 3.87E−07 mM and 3.87E−02 mM;
   xxxv. myo-Inositol at a concentration between 2.00E−03 mM and 2.00E+02 mM;
   xxxvi. Glucose at a concentration between 2.22E−01 mM and 2.22E+04 mM;
   xxxvii. Human Serum Albumin (HSA) at a concentration between 1.50E−05 mM and 1.50E+00 mM;
   xxxviii. Biotin at a concentration between 8.10E−06 mM and 8.10E−01 mM;
   xxxix. Folic Acid at a concentration between 2.40E−04 mM and 2.40E+01 mM;
   xL. Vitamin B12 at a concentration between 1.00E−02 mM and 1.00E+03 mM;
   xLi. Vitamin C at a concentration between 2.84E−03 mM and 2.84E+02 mM;
   xLii. Vitamin E at a concentration between 2.40E−03 mM and 2.40E+02 mM;
   xLiii. Zinc at a concentration between 1.50E−08 mM and 1.50E−03 mM;
   xLiv. L-Ornithine at a concentration between 5.00E−04 mM and 5.00E+01 mM;
   xLv. Creatine Anhydrous at a concentration between 1.00E−02 mM and 1.00E+03 mM;
   xLvi. PolyVinyl Alcohol (PVA) at a concentration between 1.16E−01 and 1.16E+04 mM;
   xLvii. Ethanolamine at a concentration between 5.00E−04 and 5.00E+01 mM;
   xLviii. 2-mercaptoethanol (2-ME) at a concentration between 1.00E−03 mM and 1.00E+02 mM;
   xLix. Androstenedione at a concentration between 1.00E−05 and 1.00E+00 mM;
   L. Dextran at a concentration between 1.00E+01 and 1.00E+06 mg/L;
   Li. L-Alanyl-L-Glutamine (Glutamax) at a concentration between 2.50E−02 mM and 2.50E+03 mM;
   Lii. a polyamine supplement at a concentration between 0.01× and 1000× of a 1000× stock (polyamine supplement being Sigma-Aldrich Catalog #P8483 or equivalent); and
   Liii. an antioxidant supplement at a concentration between 0.01× and 1000× of a 1000× (antioxidant supplement being Sigma-Aldrich Catalog #A1345 or equivalent); and
b. a second media formulation comprising the first media formulation and at least three of the following components:
   Liv. Nicotinamide at a concentration between 2.50E−02 mM and 2.50E+03 mM;
   Lv. IGF-1 (Insulin like Growth Factor 1) at a concentration between 2.60E−08 mM and 2.60E−03 mM;
   Lvi. 4-1BBL [4-1BB Ligand or ligand for Cluster of Differentiation 137 (CD137)] at a concentration between 4.20E−09 mM and 4.20E−04 mM;
   Lvii. Ashwagandha (botanical name: Withania Somnifera) plant extract at a concentration between 6.00E−01 mg/L and 6.00E+04 mg/L;
   Lviii. Kumquat (botanical name: Fortunella Japonica) plant extract at a concentration between 1.40E−22 mg/L and 1.40E−02 mg/L;
   Lix. Umckaloabo (botanical name: Pelargonium sidoides) plant extract at a concentration between 5.00E−01 mg/L and 5.00E+05 mg/L;
   Lx. Echinacea (botanical name: Echinacea Purpurea) plant extract at a concentration between 1.01E−01 mg/L and 1.00E+05 mg/L;
   Lxi. Red Ginseng (botanical name: Panax ginseng) plant extract at a concentration between 8.43E−02 mg/L and 8.43E+03 mg/L;
   Lxii. 2,3-Butanediol at a concentration between 9.01E−02 mg/L and 9.01E+03 mg/L; and
   Lxiii. Elderberry (botanical name: Sambucus nigra) plant extract at a concentration between 1.06E−01 mg/L and 1.06E+04 mg/L.

In certain embodiments, the NK cells or precursor cells thereof are cultured in the first media formulation.

In certain embodiments, the NK cells or precursor cells thereof are cultured in the second media formulation.

In certain embodiments, the NK cells or precursor cells thereof are cultured in the first media formulation followed by the second formulation.

In certain embodiments, the NK cells or precursor cells thereof are cultured ex vivo.

In certain embodiments, the NK cells or precursor cells thereof are cultured in vitro.

In certain embodiments, cytotoxic activity of the NK cells against target cells is enhanced.

In certain embodiments, the target cells are cancer cells.

In certain embodiments, activation of the NK cells is enhanced.

In certain embodiments, dysfunction of the NK cells is reduced.

In certain embodiments, the dysfunction is exhaustion.

In certain embodiments, metabolic function of the NK cells is enhanced.

In certain embodiments, high viability and cell-proliferation of NK cells is achieved.

In certain embodiments, use of serum is eliminated or reduced.

In certain embodiments, use of cytokine supplements is eliminated or reduced.

In certain embodiments, the use of Antigen Presenting Cells (APCs) or feeder cells is eliminated or reduced.

In certain embodiments, the culture of NK precursor cells induces their differentiation.

In certain embodiments, the culture of NK cells or precursor cells modulates the distribution and relative populations of NK cell subsets.

In certain embodiments, the NK cell subsets are memory NK cells.

In certain embodiments, the NK cell subsets are defined by relative expression of activating and inhibitory receptors.

In another aspect, the invention includes a method for cryopreservation of Natural Killer (NK) cells, wherein freezing the NK cells is accomplished in a cryopreservation medium comprising:
a. a first media formulation comprising at least six of the following components:
   i. Glycine at a concentration between 3.50E−03 mM and 3.50E+02 mM;
   ii. L-Alanine at a concentration between 1.10E−02 mM and 1.10E+03 mM;
   iii. L-Asparagine at a concentration between 1.50E−03 mM and 1.50E+02 mM;
   iv. L-Aspartic acid at a concentration between 1.50E−03 mM and 1.50E+02 mM;
   v. L-Glutamic Acid at a concentration between 1.50E−03 mM and 1.50E+02 mM;
   vi. L-Proline at a concentration between 2.50E−03 mM and 2.50E+02 mM;
   vii. L-Serine at a concentration between 3.50E−03 mM and 3.50E+02 mM;
   viii. L-Arginine HCl at a concentration between 1.30E−02 mM and 1.30E+03 mM;
   ix. L-Cystine at a concentration between 2.00E−03 mM and 2.00E+02 mM;
   x. L-Histidine HCl—H₂O at a concentration between 3.50E−03 mM and 3.50E+02 mM;
   xi. L-Isoleucine at a concentration between 8.20E−03 mM and 8.20E+02 mM;
   xii. L-Leucine at a concentration between 8.50E−03 mM and 8.50E+02 mM;
   xiii. L-Lysine hydrochloride at a concentration between 8.96E−03 mM and 8.96E+02 mM;
   xiv. L-Methionine at a concentration between 2.21E−03 mM and 2.21E+02 mM;
   xv. L-Phenylalanine at a concentration between 4.20E−03 mM and 4.20E+02 mM;
   xvi. L-Threonine at a concentration between 8.50E−03 mM and 8.50E+02 mM;
   xvii. L-Tryptophan at a concentration between 9.00E−04 mM and 9.00E+01 mM;
   xviii. L-Tyrosine at a concentration between 4.09E−03 mM and 4.09E+02 mM;
   xix. L-Valine at a concentration between 8.50E−03 mM and 8.50E+02 mM;
   xx. Sodium Pyruvate at a concentration between 1.00E−02 mM and 1.00E+03 mM;
   xxi. Arachidonic acid at a concentration between 3.28E−07 mM and 3.28E−02 mM;
   xxii. Linoleic acid at a concentration between 3.78E−06 mM and 3.78E−01 mM;
   xxiii. Linolenic acid at a concentration between 1.80E−06 mM and 1.80E−01 mM;
   xxiv. Myristic acid at a concentration between 2.19E−06 mM and 2.19E−01 mM;
   xxv. Oleic acid at a concentration between 1.77E−06 mM and 1.77E−01 mM;
   xxvi. Palmitic acid at a concentration between 1.95E−06 mM and 1.95E−01 mM;
   xxvii. Stearic acid at a concentration between 1.76E−06 mM and 1.76E−01 mM;
   xxviii. Cholesterol at a concentration between 2.84E−05 mM and 2.84E+00 mM;
   xxix. Tween-80 at a concentration between 8.40E−05 mM and 8.40E+00 mM;
   xxx. Tocopherol acetate at a concentration between 7.40E−06 mM and 7.40E−01 mM;
   xxxi. Pluronic F-68 at a concentration between 5.95E−04 mM and 5.95E+01 mM;
   xxxii. Insulin at a concentration between 1.72E−05 mM and 1.72E+00 mM;
   xxxiii. Transferrin at a concentration between 6.88E−07 mM and 6.88E−02 mM;
   xxxiv. Sodium Selenite at a concentration between 3.87E−07 mM and 3.87E−02 mM;
   xxxv. myo-Inositol at a concentration between 2.00E−03 mM and 2.00E+02 mM;
   xxxvi. Glucose at a concentration between 2.22E−01 mM and 2.22E+04 mM;
   xxxvii. Human Serum Albumin (HSA) at a concentration between 1.50E−05 mM and 1.50E+00 mM;
   xxxviii. Biotin at a concentration between 8.10E−06 mM and 8.10E−01 mM;
   xxxix. Folic Acid at a concentration between 2.40E−04 mM and 2.40E+01 mM;
   xL. Vitamin B12 at a concentration between 1.00E−02 mM and 1.00E+03 mM;
   xLi. Vitamin C at a concentration between 2.84E−03 mM and 2.84E+02 mM;
   xLii. Vitamin E at a concentration between 2.40E−03 mM and 2.40E+02 mM;
   xLiii. Zinc at a concentration between 1.50E−08 mM and 1.50E−03 mM;
   xLiv. L-Ornithine at a concentration between 5.00E−04 mM and 5.00E+01 mM;
   xLv. Creatine Anhydrous at a concentration between 1.00E−02 mM and 1.00E+03 mM;
   xLvi. PolyVinyl Alcohol (PVA) at a concentration between 1.16E−01 and 1.16E+04 mM;
   xLvii. Ethanolamine at a concentration between 5.00E−04 and 5.00E+01 mM;
   xLviii. 2-mercaptoethanol (2-ME) at a concentration between 1.00E−03 mM and 1.00E+02 mM;
   xLix. Androstenedione at a concentration between 1.00E−05 and 1.00E+00 mM;
   L. Dextran at a concentration between 1.00E+01 and 1.00E+06 mg/L;
   Li. L-Alanyl-L-Glutamine (Glutamax) at a concentration between 2.50E−02 mM and 2.50E+03 mM;
   Lii. a polyamine supplement at a concentration between 0.01× and 1000× of a 1000× stock (polyamine supplement being Sigma-Aldrich Catalog #P8483 or equivalent); and
   Liii. an antioxidant supplement at a concentration between 0.01× and 1000× of a 1000× (antioxidant supplement being Sigma-Aldrich Catalog #A1345 or equivalent); and
b. a second media formulation comprising the first media formulation and at least three of the following components:
   Liv. Nicotinamide at a concentration between 2.50E−02 mM and 2.50E+03 mM;
   Lv. IGF-1 (Insulin like Growth Factor 1) at a concentration between 2.60E−08 mM and 2.60E−03 mM;
   Lvi. 4-1BBL [4-1BB Ligand or ligand for Cluster of Differentiation 137 (CD137)] at a concentration between 4.20E−09 mM and 4.20E−04 mM;
   Lvii. Ashwagandha (botanical name: Withania Somnifera) plant extract at a concentration between 6.00E−01 mg/L and 6.00E+04 mg/L;
   Lviii. Kumquat (botanical name: Fortunella Japonica) plant extract at a concentration between 1.40E−22 mg/L and 1.40E−02 mg/L;
   Lix. Umckaloabo (botanical name: Pelargonium sidoides) plant extract at a concentration between 5.00E−01 mg/L and 5.00E+05 mg/L;
   Lx. Echinacea (botanical name: Echinacea Purpurea) plant extract at a concentration between 1.01E−01 mg/L and 1.00E+05 mg/L;
   Lxi. Red Ginseng (botanical name: Panax ginseng) plant extract at a concentration between 8.43E−02 mg/L and 8.43E+03 mg/L;
   Lxii. 2,3-Butanediol at a concentration between 9.01E−02 mg/L and 9.01E+03 mg/L; and
   Lxiii. Elderberry (botanical name: Sambucus nigra) plant extract at a concentration between 1.06E−01 mg/L and 1.06E+04 mg/L.

In certain embodiments, the cryopreservation medium comprises the first media formulation.

In certain embodiments, the cryopreservation medium comprises the second media formulation.

In another aspect, the invention includes a method for transduction of Natural Killer (NK) immune cells comprising
a. a first cell culture step comprising culturing the NK cells in a cell culture medium comprising:
   a first media formulation comprising at least six of the following components:
      i. Glycine at a concentration between 3.50E−03 mM and 3.50E+02 mM;
      ii. L-Alanine at a concentration between 1.10E−02 mM and 1.10E+03 mM;
      iii. L-Asparagine at a concentration between 1.50E−03 mM and 1.50E+02 mM;
      iv. L-Aspartic acid at a concentration between 1.50E−03 mM and 1.50E+02 mM;
      v. L-Glutamic Acid at a concentration between 1.50E−03 mM and 1.50E+02 mM;
      vi. L-Proline at a concentration between 2.50E−03 mM and 2.50E+02 mM;
      vii. L-Serine at a concentration between 3.50E−03 mM and 3.50E+02 mM;
      viii. L-Arginine HCl at a concentration between 1.30E−02 mM and 1.30E+03 mM;
      ix. L-Cystine at a concentration between 2.00E−03 mM and 2.00E+02 mM;
      x. L-Histidine HCl—H₂O at a concentration between 3.50E−03 mM and 3.50E+02 mM;
      xi. L-Isoleucine at a concentration between 8.20E−03 mM and 8.20E+02 mM;
      xii. L-Leucine at a concentration between 8.50E−03 mM and 8.50E+02 mM;
      xiii. L-Lysine hydrochloride at a concentration between 8.96E−03 mM and 8.96E+02 mM;
      xiv. L-Methionine at a concentration between 2.21E−03 mM and 2.21E+02 mM;
      xv. L-Phenylalanine at a concentration between 4.20E−03 mM and 4.20E+02 mM;
      xvi. L-Threonine at a concentration between 8.50E−03 mM and 8.50E+02 mM;
      xvii. L-Tryptophan at a concentration between 9.00E−04 mM and 9.00E+01 mM;
      xviii. L-Tyrosine at a concentration between 4.09E−03 mM and 4.09E+02 mM;
      xix. L-Valine at a concentration between 8.50E−03 mM and 8.50E+02 mM;
      xx. Sodium Pyruvate at a concentration between 1.00E−02 mM and 1.00E+03 mM;
      xxi. Arachidonic acid at a concentration between 3.28E−07 mM and 3.28E−02 mM;
      xxii. Linoleic acid at a concentration between 3.78E−06 mM and 3.78E−01 mM;
      xxiii. Linolenic acid at a concentration between 1.80E−06 mM and 1.80E−01 mM;
      xxiv. Myristic acid at a concentration between 2.19E−06 mM and 2.19E−01 mM;
      xxv. Oleic acid at a concentration between 1.77E−06 mM and 1.77E−01 mM;
      xxvi. Palmitic acid at a concentration between 1.95E−06 mM and 1.95E−01 mM;
      xxvii. Stearic acid at a concentration between 1.76E−06 mM and 1.76E−01 mM;
      xxviii. Cholesterol at a concentration between 2.84E−05 mM and 2.84E+00 mM;
      xxix. Tween-80 at a concentration between 8.40E−05 mM and 8.40E+00 mM;
      xxx. Tocopherol acetate at a concentration between 7.40E−06 mM and 7.40E−01 mM;
      xxxi. Pluronic F-68 at a concentration between 5.95E−04 mM and 5.95E+01 mM;
      xxxii. Insulin at a concentration between 1.72E−05 mM and 1.72E+00 mM;
      xxxiii. Transferrin at a concentration between 6.88E−07 mM and 6.88E−02 mM;
      xxxiv. Sodium Selenite at a concentration between 3.87E−07 mM and 3.87E−02 mM;
      xxxv. myo-Inositol at a concentration between 2.00E−03 mM and 2.00E+02 mM;
      xxxvi. Glucose at a concentration between 2.22E−01 mM and 2.22E+04 mM;
      xxxvii. Human Serum Albumin (HSA) at a concentration between 1.50E−05 mM and 1.50E+00 mM;
      xxxviii. Biotin at a concentration between 8.10E−06 mM and 8.10E−01 mM;
      xxxix. Folic Acid at a concentration between 2.40E−04 mM and 2.40E+01 mM;
      xL. Vitamin B12 at a concentration between 1.00E−02 mM and 1.00E+03 mM;
      xLi. Vitamin C at a concentration between 2.84E−03 mM and 2.84E+02 mM;
      xLii. Vitamin E at a concentration between 2.40E−03 mM and 2.40E+02 mM;
      xLiii. Zinc at a concentration between 1.50E−08 mM and 1.50E−03 mM;
      xLiv. L-Ornithine at a concentration between 5.00E−04 mM and 5.00E+01 mM;
      xLv. Creatine Anhydrous at a concentration between 1.00E−02 mM and 1.00E+03 mM;
      xLvi. PolyVinyl Alcohol (PVA) at a concentration between 1.16E−01 and 1.16E+04 mM;
      xLvii. Ethanolamine at a concentration between 5.00E−04 and 5.00E+01 mM;
      xLviii. 2-mercaptoethanol (2-ME) at a concentration between 1.00E−03 mM and 1.00E+02 mM;
      xLix. Androstenedione at a concentration between 1.00E−05 and 1.00E+00 mM;
      L. Dextran at a concentration between 1.00E+01 and 1.00E+06 mg/L;
      Li. L-Alanyl-L-Glutamine (Glutamax) at a concentration between 2.50E−02 mM and 2.50E+03 mM;
      Lii. a polyamine supplement at a concentration between 0.01× and 1000× of a 1000× stock (polyamine supplement being Sigma-Aldrich Catalog #P8483 or equivalent); and
      Liii. an antioxidant supplement at a concentration between 0.01× and 1000× of a 1000× (antioxidant supplement being Sigma-Aldrich Catalog #A1345 or equivalent); and
   a second media formulation comprising the first media formulation and at least three of the following components:
      Liv. Nicotinamide at a concentration between 2.50E−02 mM and 2.50E+03 mM;
      Lv. IGF-1 (Insulin like Growth Factor 1) at a concentration between 2.60E−08 mM and 2.60E−03 mM;
      Lvi. 4-1BBL [4-1BB Ligand or ligand for Cluster of Differentiation 137 (CD137)] at a concentration between 4.20E−09 mM and 4.20E−04 mM;
      Lvii. Ashwagandha (botanical name: Withania Somnifera) plant extract at a concentration between 6.00E−01 mg/L and 6.00E+04 mg/L;
      Lviii. Kumquat (botanical name: Fortunella Japonica) plant extract at a concentration between 1.40E−22 mg/L and 1.40E−02 mg/L;
      Lix. Umckaloabo (botanical name: Pelargonium sidoides) plant extract at a concentration between 5.00E−01 mg/L and 5.00E+05 mg/L;
      Lx. Echinacea (botanical name: Echinacea Purpurea) plant extract at a concentration between 1.01E−01 mg/L and 1.00E+05 mg/L;
      Lxi. Red Ginseng (botanical name: Panax ginseng) plant extract at a concentration between 8.43E−02 mg/L and 8.43E+03 mg/L;
      Lxii. 2,3-Butanediol at a concentration between 9.01E−02 mg/L and 9.01E+03 mg/L; and
      Lxiii. Elderberry (botanical name: Sambucus nigra) plant extract at a concentration between 1.06E−01 mg/L and 1.06E+04 mg/L;
b. contacting the NK cells with a viral vector; and
c. a second cell culture step comprising culturing the transduced NK cells in a cell culture medium comprising the cell culture medium formulation of step a.

In certain embodiments, the first and second cell cultures steps are carried out in the first cell culture formulation.

In certain embodiments, the first and second cell culture steps are carried out in the second cell culture formulation.

In certain embodiments, the first cell culture step is carried out in the first cell culture formulation and the second cell culture step is carried out in the second cell culture formulation.

In certain embodiments, the vector encodes a chimeric antigen receptor (CAR).

In another aspect, the invention provides a method for differentiating Natural Killer (NK) cells from progenitor cells comprising culturing the progenitor cells in a cell culture media comprising:
a. a first media formulation comprising at least six of the following components:
   i. Glycine at a concentration between 3.50E−03 mM and 3.50E+02 mM;
   ii. L-Alanine at a concentration between 1.10E−02 mM and 1.10E+03 mM;
   iii. L-Asparagine at a concentration between 1.50E−03 mM and 1.50E+02 mM;
   iv. L-Aspartic acid at a concentration between 1.50E−03 mM and 1.50E+02 mM;
   v. L-Glutamic Acid at a concentration between 1.50E−03 mM and 1.50E+02 mM;
   vi. L-Proline at a concentration between 2.50E−03 mM and 2.50E+02 mM;
   vii. L-Serine at a concentration between 3.50E−03 mM and 3.50E+02 mM;
   viii. L-Arginine HCl at a concentration between 1.30E−02 mM and 1.30E+03 mM;
   ix. L-Cystine at a concentration between 2.00E−03 mM and 2.00E+02 mM;
   x. L-Histidine HCl—H₂O at a concentration between 3.50E−03 mM and 3.50E+02 mM;
   xi. L-Isoleucine at a concentration between 8.20E−03 mM and 8.20E+02 mM;
   xii. L-Leucine at a concentration between 8.50E−03 mM and 8.50E+02 mM;
   xiii. L-Lysine hydrochloride at a concentration between 8.96E−03 mM and 8.96E+02 mM;
   xiv. L-Methionine at a concentration between 2.21E−03 mM and 2.21E+02 mM;
   xv. L-Phenylalanine at a concentration between 4.20E−03 mM and 4.20E+02 mM;
   xvi. L-Threonine at a concentration between 8.50E−03 mM and 8.50E+02 mM;
   xvii. L-Tryptophan at a concentration between 9.00E−04 mM and 9.00E+01 mM;
   xviii. L-Tyrosine at a concentration between 4.09E−03 mM and 4.09E+02 mM;
   xix. L-Valine at a concentration between 8.50E−03 mM and 8.50E+02 mM;
   xx. Sodium Pyruvate at a concentration between 1.00E−02 mM and 1.00E+03 mM;
   xxi. Arachidonic acid at a concentration between 3.28E−07 mM and 3.28E−02 mM;
   xxii. Linoleic acid at a concentration between 3.78E−06 mM and 3.78E−01 mM;
   xxiii. Linolenic acid at a concentration between 1.80E−06 mM and 1.80E−01 mM;
   xxiv. Myristic acid at a concentration between 2.19E−06 mM and 2.19E−01 mM;
   xxv. Oleic acid at a concentration between 1.77E−06 mM and 1.77E−01 mM;
   xxvi. Palmitic acid at a concentration between 1.95E−06 mM and 1.95E−01 mM;
   xxvii. Stearic acid at a concentration between 1.76E−06 mM and 1.76E−01 mM;
   xxviii. Cholesterol at a concentration between 2.84E−05 mM and 2.84E+00 mM;
   xxix. Tween-80 at a concentration between 8.40E−05 mM and 8.40E+00 mM;
   xxx. Tocopherol acetate at a concentration between 7.40E−06 mM and 7.40E−01 mM;
   xxxi. Pluronic F-68 at a concentration between 5.95E−04 mM and 5.95E+01 mM;
   xxxii. Insulin at a concentration between 1.72E−05 mM and 1.72E+00 mM;
   xxxiii. Transferrin at a concentration between 6.88E−07 mM and 6.88E−02 mM;
   xxxiv. Sodium Selenite at a concentration between 3.87E−07 mM and 3.87E−02 mM;
   xxxv. myo-Inositol at a concentration between 2.00E−03 mM and 2.00E+02 mM;
   xxxvi. Glucose at a concentration between 2.22E−01 mM and 2.22E+04 mM;
   xxxvii. Human Serum Albumin (HSA) at a concentration between 1.50E−05 mM and 1.50E+00 mM;
   xxxviii. Biotin at a concentration between 8.10E−06 mM and 8.10E−01 mM;
   xxxix. Folic Acid at a concentration between 2.40E−04 mM and 2.40E+01 mM;
   xL. Vitamin B12 at a concentration between 1.00E−02 mM and 1.00E+03 mM;
   xLi. Vitamin C at a concentration between 2.84E−03 mM and 2.84E+02 mM;
   xLii. Vitamin E at a concentration between 2.40E−03 mM and 2.40E+02 mM;
   xLiii. Zinc at a concentration between 1.50E−08 mM and 1.50E−03 mM;
   xLiv. L-Ornithine at a concentration between 5.00E−04 mM and 5.00E+01 mM;
   xLv. Creatine Anhydrous at a concentration between 1.00E−02 mM and 1.00E+03 mM;
   xLvi. PolyVinyl Alcohol (PVA) at a concentration between 1.16E−01 and 1.16E+04 mM;
   xLvii. Ethanolamine at a concentration between 5.00E−04 and 5.00E+01 mM;
   xLviii. 2-mercaptoethanol (2-ME) at a concentration between 1.00E−03 mM and 1.00E+02 mM;
   xLix. Androstenedione at a concentration between 1.00E−05 and 1.00E+00 mM;
   L. Dextran at a concentration between 1.00E+01 and 1.00E+06 mg/L;
   Li. L-Alanyl-L-Glutamine (Glutamax) at a concentration between 2.50E−02 mM and 2.50E+03 mM;
   Lii. a polyamine supplement at a concentration between 0.01× and 1000× of a 1000× stock (polyamine supplement being Sigma-Aldrich Catalog #P8483 or equivalent); and
   Liii. an antioxidant supplement at a concentration between 0.01× and 1000× of a 1000× (antioxidant supplement being Sigma-Aldrich Catalog #A1345 or equivalent); and
b. a second media formulation comprising the first media formulation and at least three of the following components:
   Liv. Nicotinamide at a concentration between 2.50E−02 mM and 2.50E+03 mM;
   Lv. IGF-1 (Insulin like Growth Factor 1) at a concentration between 2.60E−08 mM and 2.60E−03 mM;
   Lvi. 4-1BBL [4-1BB Ligand or ligand for Cluster of Differentiation 137 (CD137)] at a concentration between 4.20E−09 mM and 4.20E−04 mM;
   Lvii. Ashwagandha (botanical name: Withania Somnifera) plant extract at a concentration between 6.00E−01 mg/L and 6.00E+04 mg/L;
   Lviii. Kumquat (botanical name: Fortunella Japonica) plant extract at a concentration between 1.40E−22 mg/L and 1.40E−02 mg/L;
   Lix. Umckaloabo (botanical name: Pelargonium sidoides) plant extract at a concentration between 5.00E−01 mg/L and 5.00E+05 mg/L;
   Lx. Echinacea (botanical name: Echinacea Purpurea) plant extract at a concentration between 1.01E−01 mg/L and 1.00E+05 mg/L;
   Lxi. Red Ginseng (botanical name: Panax ginseng) plant extract at a concentration between 8.43E−02 mg/L and 8.43E+03 mg/L;
   Lxii. 2,3-Butanediol at a concentration between 9.01E−02 mg/L and 9.01E+03 mg/L; and
   Lxiii. Elderberry (botanical name: Sambucus nigra) plant extract at a concentration between 1.06E−01 mg/L and 1.06E+04 mg/L.

In certain embodiments, the culture media comprises the first media formulation.

In certain embodiments, the culture media comprises the second media formulation.

In certain embodiments, the culture media comprises the first media formulation followed by the second media formulation.

In certain embodiments, the culture of Natural Killer (NK) cells modulates the distribution and relative populations of NK cell subsets.

In certain embodiments, the NK cell subsets are memory NK cells.

In certain embodiments, the NK cell subsets are defined by relative expression of activating and inhibitory receptors.

### BRIEF DESCRIPTION OF THE DRAWINGS

The following detailed description of specific embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1 is a pictorial representation of a two-part media formulation, each part with a specific list of ingredients and their concentration ranges per Table-01, according to an embodiment of the present invention.

FIG. 2 is a pictorial representation of a two-part media formulation, each part created by the addition of a supplement to off-the-shelf media, where the supplements are formulated with a specific list of ingredients and their concentration ranges per Table-01, according to a further embodiment of the present invention.

FIG. 3 is a process flowchart illustrating the steps representing examples for using a two-part media formulation from FIG. 01 or FIG. 02, where either one or both parts of the two-part media formulation are used.

FIGS. 4A-4B illustrate the toxicity of primary and cell line NK cells grown in the media formulation of the invention. FIG. 4A corresponds to the cytotoxicity observed in target K562 leukemia cells after overnight incubation with effector KHYG1 cells, at an Effector: Target (E: T) ratio of 5:1. FIG. 4B corresponds to the cytotoxicity observed in target K562 leukemia cells after overnight incubation with effector primary NK cells (from a healthy adult donor), at an Effector:Target (E:T) ratio of 5:1. In both cases, the Control media is DMEM-F12, and the measured cytotoxicity is natural cytotoxicity alone (without ADCC, Antibody Dependent Cell-mediated Cytotoxicity). The notation used for statistical significance is as follows (unpaired t-test): ns is not significant, *p<0.05, **p<0.01,***p<0.001,****p<0.0001.

FIGS. 5A-5B illustrate that cytotoxicity of primary human NK cells expanded in Enable-NK™ media consistently outperformed NK cells from the same donor expanded in Control media, irrespective of whether the comparison is made without (FIG. 5A) or with (FIG. 5B) the presence of anti-GD2 antibody (for ADCC, Antibody Dependent Cell-mediated Cytotoxicity). Target cells were CHLA-20-GFP (human pediatric neuroblastoma cell line), at an Effector: Target (E:T) ratio of 5:1 (NK:CHLA-20) [co-culture in DMEM media]. Target cell death was determined by loss of GFP signal over time. Error bars indicate standard error of mean (SEM).

FIGS. 6A-6B illustrate that the Cytotoxicity of primary human NK cells expanded in Enable-NK™ media consistently outperformed NK cells from the same donor expanded in Control media, irrespective of whether the comparison is made without (FIG. 6A) or with (FIG. 6B) the presence of anti-GD2 antibody (for ADCC, Antibody Dependent Cell-mediated Cytotoxicity). Target cells were M21-GFP (human Melanoma cell line), at an Effector: Target (E: T) ratio of 5:1 (NK:M21) [co-culture in DMEM media]. Target cell death was determined by loss of GFP signal over time. All error bars indicate standard error of the mean (SEM).

FIGS. 7A-7B depict a comparison of the levels of interferon-gamma (IFN-γ) measured in the supernatant samples of Control media and Enable-NK media, following incubation of effector NK cells either cultured alone, or co-cultured with target K562 cells. NK cells secrete IFN-γ to recruit other immune cells against disease. FIG. 7A corresponds to IFN-γ levels measured with KHYG1 NK cells, and FIG. 7B corresponds to IFN-γ levels measured with primary NK cells from a healthy adult donor, with the Effector:Target (E:T) ratio of 5:1 following overnight incubation. Bars indicate Standard Error of Mean (SEM). The notation used for statistical significance is as follows (unpaired t-test): ns is not significant, *p<0.05, **p<0.01,***p<0.001,****p<0.0001.

FIGS. 8A-8D illustrate flow cytometry quantification of cell surface biomarkers on primary NK cells corresponding to NK cell activation i.e. [FIG. 8A] NKG2D [FIG. 8B] NKp46 [FIG. 8C] CD69 [FIG. 8D] CD16. Graphs represent cell counts (y-axis) and Fluorescence (x-axis). The notation used for statistical significance is as follows (unpaired t-test): ns is not significant, *p<0.05, **p<0.01,***p<0.001,****p<0.0001.

FIG. 9 illustrates the percentage (y-axis) of target cells (K562-GFP), and percentage (y-axis) of target cells (K562-GFP) which are dead, both as a function of time (x-axis), after the co-culture of KHYG-1 effector cells was set up with K562-GFP target cells at an Effector: Target ratio of 1:1 on Day-0. Bars indicate Standard Error of Mean. Notation used for statistical significance (unpaired t-test): ns is not significant, *p<0.05, **p<0.01, ***p<0.001, ****p<0.0001.

FIG. 10 illustrates the Mean Fluorescence Intensity comparison between the biomarker levels on KHYG-1 NK cells—at various points in the experiment (day 02, 10 and in both monoculture and co-culture experimental conditions). The notation used for statistical significance is as follows (unpaired t-test): ns is not significant, *p<0.05, **p<0.01,***p<0.001,****p<0.0001 FIG. 11 illustrates the percentage (y-axis) of target cells (K562-GFP), and percentage (y-axis) of target cells (K562-GFP) which are dead, both as a function of time (x-axis), after the co-culture of KHYG-1 effector cells was set up with K562-GFP target cells at an Effector: Target ratio of 1:1 on Day-0. Bars indicate Standard Error of Mean. Notation used for statistical significance (paired t-test): ns is not significant, *p<0.05, **p<0.01, ***p<0.001, ****p<0.0001.

FIG. 12 illustrates the Mean Fluorescence Intensity comparison between the biomarker levels on primary NK cells, at day-03 in both monoculture and co-culture experimental conditions. The represented data is for 3 healthy adult donors of primary NK cells, paired donor-wise between the two media conditions. The notation used for statistical significance is as follows (ratio paired t-test): ns (p-value) is not significant, *p<0.05, **p<0.01,***p<0.001,****p<0.0001.

FIG. 13 illustrates a comparison of the CD56^{dim}CD16⁺CD57⁺ sub-population between the two media conditions at day-03, as evaluated within primary NK cells (CD3-CD56+) from 3 healthy adult donors (data paired donor wise). The notation used for statistical significance (paired t-test) is as follows: ns (p-value) is not significant, *p<0.05, **p<0.01.

FIG. 14 illustrates a comparison of the CD56^{dim}CD57⁺NKG2A⁻ sub-population between the two media conditions at day-03, as evaluated within primary NK cells (CD3-CD56+) from 3 healthy adult donors (data paired donor wise). The notation used for statistical significance (paired t-test) is as follows: ns (p-value) is not significant, *p<0.05, **p<0.01.

FIG. 15 illustrates a comparison of the CD56_{dim}CD16+sub-population between the two media conditions at day-03, as evaluated within primary NK cells (CD3-CD56+) from 3 healthy adult donors (data paired donor wise). Notation used for statistical significance (paired t-test): ns is not significant, *p<0.05, **p<0.01, ***p<0.001, ****p<0.0001.

FIG. 16 illustrates a comparison of the CD56^{dim} sub-population between the two media conditions at day-03, as evaluated within primary NK cells (CD3-CD56+) from 3 healthy adult donors (data paired donor wise). Notation used for statistical significance (paired t-test): ns (p-value) is not significant, *p<0.05, **p<0.01, ***p<0.001, ****p<0.0001.

FIG. 17 illustrates that normalized NAD values were more than 3-fold higher for KHYG1 NK cells in the Enable-NK media conditions. Bars indicate standard deviation. The notation used for statistical significance (unpaired t-test) is as follows: *p<0.05, **p<0.01.

FIG. 18 illustrates a comparison of the efficiency of transduction on NK-92 cells in a-MEM medium (21.0%) compared to Enable-NK Part-II medium (62.8%), as confirmed by the double-staining of both NKp-44 and FLAG. In contrast, the parental cells displayed no significant staining (<1%).

FIG. 19 illustrates the number of CHLA-20-GFP target cancer cells per well of the co-culture with NK cells—as a function of time based on the microscopy observations from the Sartorius Incucyte instrument. Greater CHLA-20-GFP target cell cytotoxicity was observed for Enable-NK media vs Control media (both with and without anti-GD2 Ab). Error bars are Standard Error of Mean.

FIG. 20 illustrates the fold expansion achieved by Enable-NK media (various Parts), when NK cells were cultured ex vivo in the absence of any feeder cells, and at reduced serum levels in comparison to Control media: [A] NK cell line NK3.3 [B] NK cell line KHYG1 and [C] primary human NK cells (from a healthy adult donor). Error bars indicate Standard Error of Mean (SEM)

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used. It is also to be understood that the terminology used herein is for the purpose of describing particular embodiments only and is not intended to be limiting. The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or +10%, more preferably ±5%, even more preferably ±1%, and still more preferably ±0.1% from the specified value, as such variations are appropriate to perform the disclosed methods.

“Base Media” as used herein is interchangeable with “off the shelf media”, “starting media”, and “underlying media”, examples include but are not limited to: DMEM/F12 (Dulbecco's Modified Eagle Medium/Nutrient Mixture F-12), RPMI-1640 (RPMI: Roswell Park Memorial Institute), MEM (Minimum Essential Media), and IMDM (Iscove's Modified Dulbecco's Medium).

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to, anti-tumor activity as determined by any means suitable in the art.

The term “cell culture” or “culturing”, as used herein, refers to the maintenance of cells (e.g. NK cells) under controlled conditions, generally outside their natural environment. Cells can be cultured to expand and maintain a population of cells for subsequent therapeutic, industrial, or scientific use. Culturing of cells can be a key part of other processes involving live cells, including but not limited to cryopreservation and engineering or otherwise manipulating cell function by inserting exogenous nucleic acids through, for example, transduction or transfection.

The term “exhaustion” or “immune exhaustion”, as used herein, refers to a functional state of immune cells (e.g. NK cells) which is characterized by impaired or reduced proliferative activity, cytokine production, and/or cytotoxic activity. Exhaustion may be caused by repeated stimulation, signaling through inhibitory signaling receptors, depletion of certain nutrients in the immediate microenvironment, or a combination of all of these factors. Due to the functional impairment, the onset of exhaustion limits the clinical effectiveness of therapeutic immune cells, including NK cells.

As used herein, an “instructional material” includes a publication, a recording, a diagram, or any other medium of expression which can be used to communicate the usefulness of the compositions and methods of the invention. The instructional material of the kit of the invention may, for example, be affixed to a container which contains the nucleic acid, peptide, and/or composition of the invention or be shipped together with a container which contains the nucleic acid, peptide, and/or composition. Alternatively, the instructional material may be shipped separately from the container with the intention that the instructional material and the compound be used cooperatively by the recipient.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine. Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

As used herein “Enable-NK media” refers to the media of the present invention as defined by the claims.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The invention provided by the current disclosure includes unique media formulation(s) for the ex vivo or in vitro culturing or genetic modification or preservation of Natural Killer (NK) cells, thus enabling the generation of more potent and better performing NK cellular therapies. The new media formulation(s) incorporate a novel combination of ingredients at specific concentration ranges to create a single-part or a multi-part media for the ex vivo culturing or genetic modification or preservation of NK cells. The present invention is described in enabling detail in the following examples, which may represent more than one embodiment of the present invention.

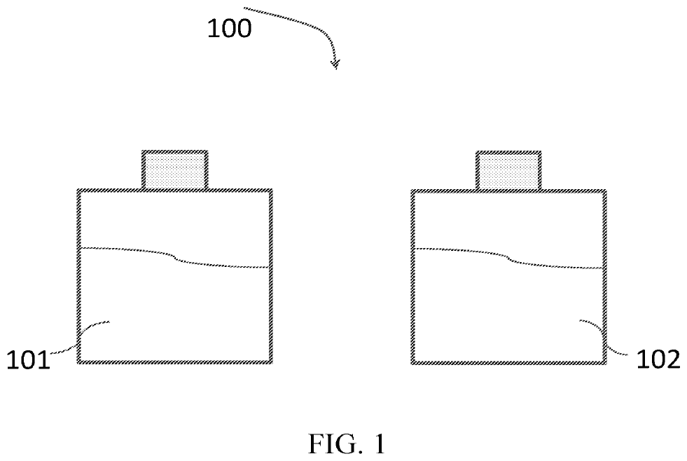

FIG. 1 is a pictorial representation of a two-part media formulation, each part with a specific list of ingredients and their concentration ranges per Table-01, according to an embodiment of the present invention, which is also the preferred embodiment. Media formulation 100 is in this example a two-part media for the culturing of NK cells. In this example, the two-part media formulation 100 has the first part 101, which is formulated using the ingredient list from the “Part 01 sub-formulation” section of Table-01, with each ingredient at a specified concentration range, as listed under “Part 01 sub-formulation” in Table-01. In this example, the two-part media formulation 100 has the second part 102, which is formulated using the ingredient list from the “Part 02 sub-formulation” section of Table-01, with each ingredient at a specified concentration range, as listed under “Part 02 sub-formulation” in Table-01.

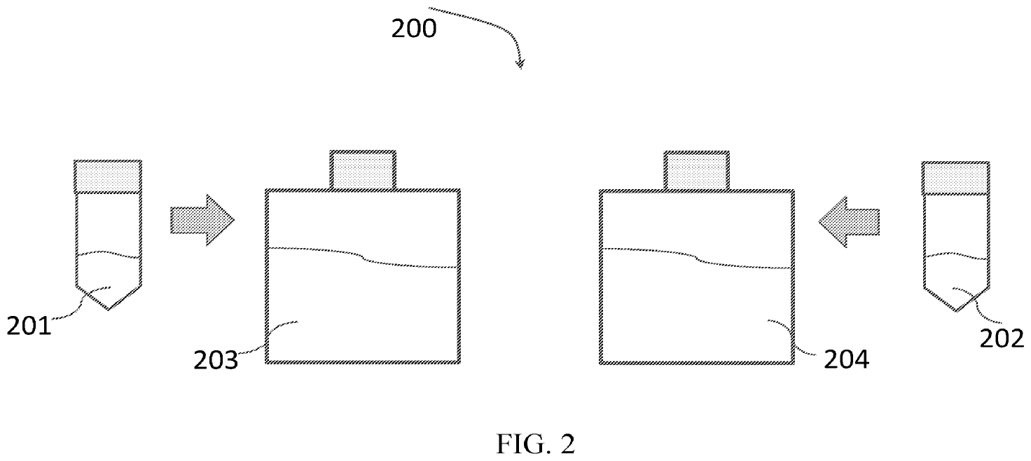

FIG. 02 is a pictorial representation of a two-part media formulation, each part created by the addition of a supplement to off-the-shelf media, where the supplements are formulated with a specific list of ingredients and their concentration ranges per Table-01, according to a further embodiment of the present invention. Media formulation 200 is in this example a two-part media for the culturing of NK cells, where part 201 is a supplement formulated using the ingredient list from the “Part 01 sub-formulation” section of Table-01, with each ingredient at a specified concentration range, as listed under “Part 01 sub-formulation” in Table-01, and where part 202 is a supplement formulated using the ingredient list from the “Part 02 sub-formulation” section of Table-01, with each ingredient at a specified concentration range, as listed under “Part 02 sub-formulation” in Table-01. In this example, the two-part media formulation 200 has a first part which is created by the addition of part 201, a supplement to part 203, an off-the-shelf media. In this example, the two-part media formulation 200 has a second part which is created by the addition of part 202, a supplement to part 204, an off-the-shelf media. In this example, “off-the-shelf” media refers to any commercial or non-commercial media formulation, such as DMEM/F12 (Dulbecco's Modified Eagle Medium/Nutrient Mixture F-12), RPMI-1640 (RPMI: Roswell Park Memorial Institute), MEM (Minimum Essential Media), IMDM (Iscove's Modified Dulbecco's Medium) and others.

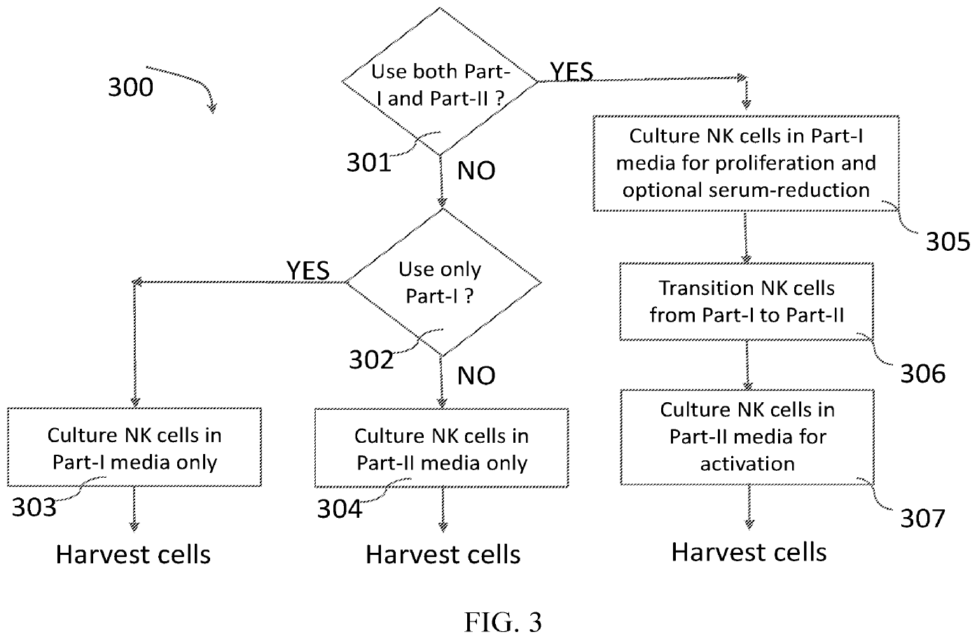

FIG. 03 is a process flowchart illustrating the steps representing examples for using a two-part media formulation from FIG. 01 or FIG. 02, where either one or both parts of the two-part media formulation are used.

When in operation to culture NK cells in one example of the process illustrated in FIG. 03, the two parts of a two-part media formulation are used in sequence, with the first part medium used for the proliferation of NK cells along with the option of serum-reduction or complete serum-elimination (i.e. serum-free media). Subsequently, the cultured NK cells are transitioned between the first part medium and the second part medium of the two-part media formulation by centrifugation, dilution of the first part medium by the second part medium, or other means. Subsequently, the second part medium is used for the culturing of NK cells for their activation.

When in operation to culture NK cells in another example of the process illustrated in FIG. 03, the first part medium of the two-part media formulation is used without the use of the second part medium. The first part medium is used for the proliferation of NK cells along with the option of serum-reduction or complete serum-elimination (i.e. serum-free media). The first part medium is also used for the culturing of NK cells for their activation.

When in operation to culture NK cells in yet another example of the process illustrated in FIG. 03, the second part medium of the two-part media formulation is used without the use of the first part medium. The second part medium is used for the proliferation of NK cells along with the option of serum-reduction or complete serum-elimination (i.e. serum-free media). The second part medium is also used for the culturing of NK cells for their activation.

The inventors provide a unique cell culture medium formulation for Natural Killer (NK) immune cells that facilitates more robust NK cell cytotoxic activity, while also supporting cell-maintenance and cell-proliferation at reduced serum levels in the medium. The above-described embodiments and examples of the invention are intended to optimally achieve desirable attributes of NK cellular therapies (e.g. serum-reduction, proliferation, higher cytotoxicity resulting from activation) through the use of individual versions of the media formulation (i.e. sub-formulations) that are optimized for specific desirable attribute(s) of the resulting of NK cellular therapies, while other desirable attribute(s) are also supported. As an example, the inventors provide unique cell culture media formulations for Natural Killer (NK) immune cells which facilitate more robust NK cell cytotoxic activity, while also supporting cell-maintenance and cell-proliferation at reduced serum levels in the media—by virtue of the specific combinations of individual ingredients which were optimized, as well as the specific concentration ranges of individual ingredients which were optimized in the media formulations, as represented by Table-01 below.

TABLE 01
Tabular summary of the ingredients and their concentration ranges for each sub-formulation
including the Part-01 sub-formulation, Part-02 sub-formulation (added on top of
the Part-01 sub-formulation ingredients), and the Part-03 sub-formulation (added
on top of the Part-01 and the Part-02 sub-formulation ingredients)

| | | Primary | | Secondary | | Tertiary | |
|---|---|---|---|---|---|---|---|
| Ingredient | Unit | Low | High | Low | High | Low | High |

Ingredient combination and concentrations for Part-01 sub-formulation

| | | | | | | | |
|---|---|---|---|---|---|---|---|
| Glycine | mM | 3.50E−03 | 3.50E+02 | 3.50E−02 | 3.50E+01 | 1.75E−01 | 7.00E+00 |
| L-Alanine | mM | 1.10E−02 | 1.10E+03 | 1.10E−01 | 1.10E+02 | 5.50E−01 | 2.20E+01 |
| L-Asparagine | mM | 1.50E−03 | 1.50E+02 | 1.50E−02 | 1.50E+01 | 7.50E−02 | 3.00E+00 |
| L-Aspartic acid | mM | 1.50E−03 | 1.50E+02 | 1.50E−02 | 1.50E+01 | 7.50E−02 | 3.00E+00 |
| L-Glutamic Acid | mM | 1.50E−03 | 1.50E+02 | 1.50E−02 | 1.50E+01 | 7.50E−02 | 3.00E+00 |
| L-Proline | mM | 2.50E−03 | 2.50E+02 | 2.50E−02 | 2.50E+01 | 1.25E−01 | 5.00E+00 |
| L-Serine | mM | 3.50E−03 | 3.50E+02 | 3.50E−02 | 3.50E+01 | 1.75E−01 | 7.00E+00 |
| L-Arginine HCl | mM | 1.30E−02 | 1.30E+03 | 1.30E−01 | 1.30E+02 | 6.50E−01 | 2.60E+01 |
| L-Cystine | mM | 2.00E−03 | 2.00E+02 | 2.00E−02 | 2.00E+01 | 1.00E−01 | 4.00E+00 |
| L-Histidine HCl—H2O | mM | 3.50E−03 | 3.50E+02 | 3.50E−02 | 3.50E+01 | 1.75E−01 | 7.00E+00 |
| L-Isoleucine | mM | 8.20E−03 | 8.20E+02 | 8.20E−02 | 8.20E+01 | 4.10E−01 | 1.64E+01 |
| L-Leucine | mM | 8.50E−03 | 8.50E+02 | 8.50E−02 | 8.50E+01 | 4.25E−01 | 1.70E+01 |
| L-Lysine hydrochloride | mM | 8.96E−03 | 8.96E+02 | 8.96E−02 | 8.96E+01 | 4.48E−01 | 1.79E+01 |
| L-Methionine | mM | 2.21E−03 | 2.21E+02 | 2.21E−02 | 2.21E+01 | 1.11E−01 | 4.43E+00 |
| L-Phenylalanine | mM | 4.20E−03 | 4.20E+02 | 4.20E−02 | 4.20E+01 | 2.10E−01 | 8.40E+00 |
| L-Threonine | mM | 8.50E−03 | 8.50E+02 | 8.50E−02 | 8.50E+01 | 4.25E−01 | 1.70E+01 |
| L-Tryptophan | mM | 9.00E−04 | 9.00E+01 | 9.00E−03 | 9.00E+00 | 4.50E−02 | 1.80E+00 |
| L-Tyrosine | mM | 4.09E−03 | 4.09E+02 | 4.09E−02 | 4.09E+01 | 2.04E−01 | 8.18E+00 |
| L-Valine | mM | 8.50E−03 | 8.50E+02 | 8.50E−02 | 8.50E+01 | 4.25E−01 | 1.70E+01 |
| Sodium Pyruvate | mM | 1.00E−02 | 1.00E+03 | 1.00E−01 | 1.00E+02 | 5.00E−01 | 2.00E+01 |
| Arachidonic acid | mM | 3.28E−07 | 3.28E−02 | 3.28E−06 | 3.28E−03 | 1.64E−05 | 6.57E−04 |
| Linoleic acid | mM | 3.78E−06 | 3.78E−01 | 3.78E−05 | 3.78E−02 | 1.89E−04 | 7.57E−03 |
| Linolenic acid | mM | 1.80E−06 | 1.80E−01 | 1.80E−05 | 1.80E−02 | 8.98E−05 | 3.59E−03 |
| Myristic acid | mM | 2.19E−06 | 2.19E−01 | 2.19E−05 | 2.19E−02 | 1.09E−04 | 4.38E−03 |
| Oleic acid | mM | 1.77E−06 | 1.77E−01 | 1.77E−05 | 1.77E−02 | 8.85E−05 | 3.54E−03 |
| Palmitic acid | mM | 1.95E−06 | 1.95E−01 | 1.95E−05 | 1.95E−02 | 9.75E−05 | 3.90E−03 |
| Stearic acid | mM | 1.76E−06 | 1.76E−01 | 1.76E−05 | 1.76E−02 | 8.79E−05 | 3.52E−03 |
| Cholesterol | mM | 2.84E−05 | 2.84E+00 | 2.84E−04 | 2.84E−01 | 1.42E−03 | 5.69E−02 |
| Tween-80 | mM | 8.40E−05 | 8.40E+00 | 8.40E−04 | 8.40E−01 | 4.20E−03 | 1.68E−01 |
| Tocopherol acetate | mM | 7.40E−06 | 7.40E−01 | 7.40E−05 | 7.40E−02 | 3.70E−04 | 1.48E−02 |
| Pluronic F-68 | mM | 5.95E−04 | 5.95E+01 | 5.95E−03 | 5.95E+00 | 2.98E−02 | 1.19E+00 |
| Insulin | mM | 1.72E−05 | 1.72E+00 | 1.72E−04 | 1.72E−01 | 8.61E−04 | 3.44E−02 |
| Transferrin | mM | 6.88E−07 | 6.88E−02 | 6.88E−06 | 6.88E−03 | 3.44E−05 | 1.38E−03 |
| Sodium Selenite | mM | 3.87E−07 | 3.87E−02 | 3.87E−06 | 3.87E−03 | 1.94E−05 | 7.75E−04 |
| myo-Inositol | mM | 2.00E−03 | 2.00E+02 | 2.00E−02 | 2.00E+01 | 1.00E−01 | 4.00E+00 |
| Glucose | mM | 2.22E−01 | 2.22E+04 | 2.22E+00 | 2.22E+03 | 1.11E+01 | 4.45E+02 |
| HSA | mM | 1.50E−05 | 1.50E+00 | 1.50E−04 | 1.50E−01 | 7.50E−04 | 3.00E−02 |
| Biotin | mM | 8.10E−06 | 8.10E−01 | 8.10E−05 | 8.10E−02 | 4.05E−04 | 1.62E−02 |
| Folic Acid | mM | 2.40E−04 | 2.40E+01 | 2.40E−03 | 2.40E+00 | 1.20E−02 | 4.80E−01 |
| Vitamin B12 | mM | 1.00E−02 | 1.00E+03 | 1.00E−01 | 1.00E+02 | 5.00E−01 | 2.00E+01 |
| Vitamin C | mM | 2.84E−03 | 2.84E+02 | 2.84E−02 | 2.84E+01 | 1.42E−01 | 5.68E+00 |
| Vitamin E | mM | 2.40E−03 | 2.40E+02 | 2.40E−02 | 2.40E+01 | 1.20E−01 | 4.80E+00 |
| Zinc | mM | 1.50E−08 | 1.50E−03 | 1.50E−07 | 1.50E−04 | 7.49E−07 | 3.00E−05 |
| L-Ornithine | mM | 5.00E−04 | 5.00E+01 | 5.00E−03 | 5.00E+00 | 2.50E−02 | 1.00E+00 |
| Creatine Anhydrous | mM | 1.00E−02 | 1.00E+03 | 1.00E−01 | 1.00E+02 | 5.00E−01 | 2.00E+01 |
| PVA | mM | 1.16E−01 | 1.16E+04 | 1.16E+00 | 1.16E+03 | 5.81E+00 | 2.32E+02 |
| Ethanolamine | mM | 5.00E−04 | 5.00E+01 | 5.00E−03 | 5.00E+00 | 2.50E−02 | 1.00E+00 |
| 2-ME | mM | 1.00E−03 | 1.00E+02 | 1.00E−02 | 1.00E+01 | 5.00E−02 | 2.00E+00 |
| Androstenedione | mM | 1.00E−05 | 1.00E+00 | 1.00E−04 | 1.00E−01 | 5.00E−04 | 2.00E−02 |
| Dextran | mg/L | 1.00E+01 | 1.00E+06 | 1.00E+02 | 1.00E+05 | 5.00E+02 | 2.00E+04 |
| L-Alanyl-L-Glutamine | mM | 2.50E−02 | 2.50E+03 | 2.50E−01 | 2.50E+02 | 1.25E+00 | 5.00E+01 |
| (Glutamax) | | | | | | | |
| Polyamine supplement | × | 0.01× | 1000× | 0.1× | 100× | 0.5× | 20× |
| (Sigma Catalog # P8483 | | | | | | | |
| at 1000× concentration) | | | | | | | |
| Antioxidant supplement | × | 0.01× | 1000× | 0.1× | 100× | 0.5× | 20× |
| (Sigma Catalog # A1345 | | | | | | | |
| at 1000× concentration) | | | | | | | |

Ingredient combination and concentrations for Part-02 sub-formulation (added to Part-01)

| | | | | | | | |
|---|---|---|---|---|---|---|---|
| Nicotinamide | mM | 2.50E−02 | 2.50E+03 | 2.50E−01 | 2.50E+02 | 1.25E+00 | 5.00E+01 |
| IGF-1 | mM | 2.60E−08 | 2.60E−03 | 2.60E−07 | 2.60E−04 | 1.30E−06 | 5.20E−05 |
| 4-1BBL | mM | 4.20E−09 | 4.20E−04 | 4.20E−08 | 4.20E−05 | 2.10E−07 | 8.40E−06 |
| Ashwagandha | mg/L | 6.00E−01 | 6.00E+04 | 6.00E+00 | 6.00E+03 | 3.00E+01 | 1.20E+03 |
| Kumquat | mg/L | 1.40E−22 | 1.40E−02 | 1.40E−19 | 1.40E−03 | 1.40E−16 | 1.40E−04 |
| Umckaloabo | mg/L | 5.00E−01 | 5.00E+05 | 5.00E+00 | 5.00E+04 | 2.50E+01 | 1.00E+04 |
| Echinacea | mg/L | 1.01E−01 | 1.00E+05 | 1.01E+00 | 1.01E+04 | 5.07E+00 | 2.03E+03 |
| Red Ginseng | mg/L | 8.43E−02 | 8.43E+03 | 8.43E−01 | 8.43E+02 | 4.22E+00 | 1.69E+02 |
| 2,3-Butanediol | mg/L | 9.01E−02 | 9.01E+03 | 9.01E−01 | 9.01E+02 | 4.51E+00 | 1.80E+02 |
| Elderberry | mg/L | 1.06E−01 | 1.06E+04 | 1.06E+00 | 1.06E+03 | 5.28E+00 | 2.11E+02 |

Ingredient combination and concentrations for Part-03 sub-formulation (added to Part-02)

| | | | | | | | |
|---|---|---|---|---|---|---|---|
| Ganoderma Lucidum | mg/L | 1.00E+00 | 1.00E+05 | 1.00E+01 | 1.00E+04 | 5.00E+01 | 2.00E+03 |
| Mistletoe | mg/L | 1.70E−01 | 1.70E+04 | 1.70E+00 | 1.70E+03 | 8.50E+00 | 3.40E+02 |

In Table 01, the following acronyms are used: HSA (Human Serum Albumin), PVA (PolyVinyl Acetate, 2-ME (2-MercaptoEthanol), IGF-1 (Insulin-like Growth Factor 1), 4-1BBL (4-1BB ligand or ligand for CD137), mM (milli-Molar), mg/L (milligrams per liter). In Table 01, for plant extracts, their concentration in the preferred embodiment of the invention (as summarized in Table 01) was determined based on the equivalent dry plant extract in the final volume of media, as expressed in the mg/L concentration units. The botanical names of the plant extract mentioned in Table 01 are as follows: Ashwagandha (botanical name: Withania Somnifera, family: Solanaceae); Kumquat (botanical name: Fortunella Japonica or Citrus Japonica, family: Rutaceae); Umckaloabo (botanical name: Pelargonium sidoides, family: Geraniaceae); Echinacea (botanical name: Echinacea Purpurea, family: Asteraceae); Red Ginseng (botanical name: Panax ginseng, family: Araliaceae); Elderberry (botanical name: Sambucus nigra, family: Adoxaceae); Ganoderma Lucidum (botanical name: Ganoderma Lucidum, family: Ganodermataceae); Mistletoe (botanical name: Viscum Album, family: Santalaceae). In alternative embodiments of the invention, the use of the above-listed plant extracts may be replaced by botanical variants of the above-listed plant extracts. Also, in alternative embodiments of the invention, the use of the above-listed plant extracts may be replaced by chemically synthesized phytochemicals, or combination of phytochemicals, or subset of phytochemicals which constitute the above-listed plant extracts.

The media formulation represented by the specific ingredient combination and concentration ranges summarized in Table 01, including the Part-01 sub-formulation, Part-02 sub-formulation and Part-03 sub-formulation, were created by the addition of some of these ingredients to DMEM/F12 medium (Dulbecco's Modified Eagle Medium/Nutrient Mixture F-12). The ingredient concentration ranges summarized in Table 01 are inclusive of the concentrations of some ingredients already contributed by the underlying medium, DMEM/F12 medium (Dulbecco's Modified Eagle Medium/Nutrient Mixture F-12). Therefore, while the DMEM/F12 underlying medium was used for media formulation development, alternative media, either basic/basal or complex (e.g. RPMI1640, MEM, IMDM etc.) can also be used instead as the underlying or starting off-the-shelf media into which additional ingredients can be added to generate alternative embodiments of the invention; RPMI-1640 (RPMI: Roswell Park Memorial Institute; MEM: Minimum Essential Media; IMDM: Iscove's Modified Dulbecco's Medium. The underlying DMEM/F12 media is interchangeable with other media because the final concentrations of ingredients, including added ingredients, can be achieved to the levels outlined in Tables 01, irrespective of which underlying/off-the-shelf media is used.

Alternative embodiments of the invention include unique combinations of subsets of ingredients listed in Table 01. Alternative embodiments of the invention also include unique combinations of ingredients listed in Table 01 (either the whole list or subsets of the list), when combined with ingredients not listed in Table 01. Alternative embodiments of the invention further include a multi-part media formulation, where the number of parts is more than two, such as a 3-part media formulation. An example of a 3-part media formulation is one where the first part is formulated using the ingredient list and concentration range from the “Part 01 sub-formulation” section of Table-01, the second part is formulated using the ingredient list and concentration range from the “Part 02 sub-formulation” section of Table-01, and the third part is formulated using the ingredient list and concentration range from the “Part 03 sub-formulation” section of Table-01. In this example, the first part of the 3-part media formulation is optimized for stepwise serum-reduction or serum-elimination, while it also supports other desirable attributes of NK cellular therapies (e.g. proliferation, or activation). Also, in this example, the second part of the 3-part media formulation is optimized for NK cell proliferation, while it also supports other desirable attributes of NK cellular therapies (e.g. serum-reduction, or activation). Also, in this example, the third part of the 3-part media formulation is optimized for NK cell activation, while it also supports other desirable attributes of NK cellular therapies (e.g. proliferation, or serum-reduction). In yet other alternative embodiments of the invention, other examples of multi-part media formulations may be created, where each part of the multi-part media formulation is optimized for a specific desirable attribute of NK cellular therapies, while also supporting other desirable attributes of NK cellular therapies.

Table-02 below is a subset of Table-01, and summarizes examples of some unique and atypical media formulation ingredients, which were combined at specific concentration ranges, and added to the underlying media of DMEM/F12, as part of the comprehensive media formulation represented by Table-01. Since Table-01 is a more comprehensive list of ingredients, it includes both the atypical/non-obvious ingredients (examples in Table 02), and also some of the more conventional/typical ingredients incorporated in media formulations to culture cells. The unique combinatorial incorporation of both atypical and conventional ingredients at specific concentration ranges (Table 01), and all the variations of media formulations which can emerge out of this unique combinatorial framework (Table 01) represent alternative embodiments of the invention. For this invention, the “primary” concentration range of each individual ingredient is wider and includes the “secondary” range, which in turn includes the “tertiary” range.

TABLE 02
Tabular summary of examples of some of the atypical/non-obvious ingredients and
their concentration ranges for each sub-formulation. Listed are 5 atypical/non-
obvious example ingredients for the Part-01 sub-formulation. For the Part-02 sub-
formulation, an additional 3 example ingredients are listed. For the Part-03 sub-
formulation, an additional example atypical/non-obvious ingredient is listed

| | | Primary | | Secondary | | Tertiary | |
|---|---|---|---|---|---|---|---|
| Ingredient | Unit | Low | High | Low | High | Low | High |

Ingredient combination and concentrations of some atypical ingredients for Part-01 sub-formulation

| | | | | | | | |
|---|---|---|---|---|---|---|---|
| L-Ornithine | mM | 5.00E−04 | 5.00E+01 | 5.00E−03 | 5.00E+00 | 2.50E−02 | 1.00E+00 |
| Creatine | mM | 1.00E−02 | 1.00E+03 | 1.00E−01 | 1.00E+02 | 5.00E−01 | 2.00E+01 |
| Anhydrous | | | | | | | |
| PVA | mM | 1.16E−01 | 1.16E+04 | 1.16E+00 | 1.16E+03 | 5.81E+00 | 2.32E+02 |
| Ethanolamine | mM | 5.00E−04 | 5.00E+01 | 5.00E−03 | 5.00E+00 | 2.50E−02 | 1.00E+00 |
| Androstenedione | mM | 1.00E−05 | 1.00E+00 | 1.00E−04 | 1.00E−01 | 5.00E−04 | 2.00E−02 |
| Dextran | mg/L | 1.00E+01 | 1.00E+06 | 1.00E+02 | 1.00E+05 | 5.00E+02 | 2.00E+04 |

Ingredient combination and concentrations of some atypical ingredients for Part-02 sub-formulation

| | | | | | | | |
|---|---|---|---|---|---|---|---|
| Ashwagandha | mg/L | 6.00E−01 | 6.00E+04 | 6.00E+00 | 6.00E+03 | 3.00E+01 | 1.20E+03 |
| Echinacea | mg/L | 1.01E−01 | 1.00E+05 | 1.01E+00 | 1.01E+04 | 5.07E+00 | 2.03E+03 |
| Red Ginseng | mg/L | 8.43E−02 | 8.43E+03 | 8.43E−01 | 8.43E+02 | 4.22E+00 | 1.69E+02 |

Ingredient combination and concentrations of some atypical ingredients for Part-03 sub-formulation

| | | | | | | | |
|---|---|---|---|---|---|---|---|
| Mistletoe | mg/L | 1.70E−01 | 1.70E+04 | 1.70E+00 | 1.70E+03 | 8.50E+00 | 3.40E+02 |

Possible embodiments of the invention may be represented by various individual media formulations which can be uniquely created by incorporating a specific combination of ingredients [derived from Table-01 at specific concentration ranges for ingredients], which may constitute stand-alone media product(s) for NK cells, or specific combination(s) of supplementary ingredients that can be added to any off-the-shelf media.

To further elaborate on Alternative embodiments, the invention covers not just the specific formulation details summarized in Table 01 (combinations and concentration ranges), but also derivative and/or alternative formulations that can be based on Table 01. Examples of alternative embodiments of the invention include, but are not limited to the following:
permutations and combinations of a subset of the ingredients listed in Table 01 in order to create derivative or alternative sub-formulations for the part-01 or part-02 or part-03 media, including but not limited to: subsets of only the molecular ingredients, subsets of only the plant extract ingredients, or plant extract variants from same botanical family
permutations and combinations of the subsets of the molecular components of plant extracts, whether plant-derived or chemically synthesized, or recombinantly synthesized. Examples include but are not limited to the following: the Iscador peptide from the molecular components of Mistletoe, flavonoid molecules from Elderberry, etc.
combining ingredients listed in Table 01 (and subsets thereof) with other ingredients not listed in Table 01, e.g. molecular ingredients and plant extracts not listed in Table 01. Examples of other plant extracts/ingredients include but are not limited to: Resveratrol, Carrageenan (e.g. Lambda Carrageenan), Neem (botanical name: Azadirachta Indica), Curcuma Longa, Euphorbia Peplus, Homalanthus Nutans (Euphorbiaceae family), Phyllanthus Poilanei, Ascophyllum Nodosum, Aphanizomenon flos-aquae, Nigella Sativum, Allium Sativum, Allium Cepa, Onopordum Acanthium, as well as specific compounds derived from plant/botanical sources including but not limited to the following: Diallyl Disulfide (derived from garlic), Genistein, Curcumin, Ingenol Mebutate, Prostratin, Phyllanthusmin C, Ascophyllan, Fucoidan.
higher or lower concentrations of the ingredients and their concentration ranges listed in Table 01, where the relative ratios between the concentrations of individual ingredients are maintained.
the same combinations of ingredients as those outlined in Table 01 (or their subsets), but at concentration levels differing from those summarized in Table 01
Dried whole plant/herb or plant extracts created with a wide range of extraction methods, e.g. solvent extraction (maceration/soxhlet), or using ultrasound, microwave or supercritical fluid
Use of any one sub-formulation of the described preferred embodiment for accomplishing all of the following 3 objectives: serum-reduction, proliferation, and enhanced cytotoxicity
Formulation of the ingredients listed in Table 01 as a supplement to media, either pre-mixed and/or with individual ingredients provided separately (e.g. as part of a kit, for instance in lyophilized form)—rather than pre-formulated with the underlying media
It will be apparent to one with skill in the art that the media formulation(s) of the invention may be provided using some or all of the mentioned features and components without departing from the spirit and scope of the present invention. It will also be apparent to the skilled artisan that the embodiments described above are specific examples of a single broader invention which may have greater scope than any of the singular descriptions taught. There may be many alterations made in the descriptions without departing from the spirit and scope of the present invention.

The invention can be used for cellular immunotherapy applications, resulting in multifaceted usefulness and utility, including but not limited to the context of: [a] research use of NK cells, and [b] clinical use of NK cellular therapies (e.g. following ex vivo/bioreactor expansion). Key desirable outcomes of the invention for NK cells include but are not limited to:
[i] higher cytotoxic activity of NK cells, resulting in enhanced killing of diseased cells (e.g. cancer cells or virus-infected cells or other diseased cells), which can lead to better therapeutic outcomes. Such utility of enhanced cytotoxic activity may be imparted through the mechanisms of apoptosis or necrosis, with or without enhanced natural cytotoxicity mechanisms of NK cells or ADCC (Antigen Dependent Cell-mediated Cytotoxicity) mechanism of NK cells or amplification of the overall immune response through NK cell signaling to other immune cells.
[ii] enhanced activation of NK cells, resulting in improved cytotoxicity but also overall biological function of the NK cells, which can lead to better research and clinical outcomes.
[iii] obviation or reduction in the dependence on cytokines or chemokines in the cell culture of NK cells for their cytotoxicity or proliferation, which can result in reduced cellular exhaustion, as well as reduced cost.
[iv] obviation or reduction of APCs (Antigen Presenting Cells, also called “feeder cells”) for the proliferation (i.e. ex vivo expansion or growth) or activation of NK cells.
[v] reduced serum-dependence or adaptation of NK cells (including complete serum elimination or serum obviation, leading to serum-free media) for cell maintenance and proliferation, resulting in improved batch-to-batch consistency and lower cost of the ex vivo/bioreactor expansion of NK cells. Enhanced cell proliferation at conventional serum levels, or enhanced cell viability at a range of serum conditions are other benefits which may be leveraged.
[vi] mitigation of cellular dysfunction (e.g. exhaustion or anergy or senescence), resulting in improved in vivo persistence of NK cells, especially in the tumor microenvironment.
[vii] enhanced metabolism of NK cells, resulting in better performance in all aspects of NK cell function, including but not limited to: NK cell cytotoxicity, reduced NK cell exhaustion, effective interfacing between the innate and adaptive immune systems, or modulation of the overall immune response through immune amplification or immune homeostasis.
[viii] improved cryopreservation of NK cells, leading to improved logistics, supply chain and efficacy of the NK cellular therapeutics. Related benefits include the facilitation of direct thawing of frozen NK cells into low serum (or serum-free) media conditions. The benefits also include enhanced cell motility or migration of NK cells, which in turn positively impact cryopreservation of NK cells.
[ix] improved transduction of NK cells, for e.g. better transduction efficiency using viral vectors, resulting in genetically engineered or genetically modified NK cell therapies (e.g. CAR-NK cells; CAR: Chimeric Antigen Receptor) which are more efficacious or for which the identification of the best performing clonal CAR-NK population is facilitated by the invention.
[x] improved transfection of NK cells, for e.g. using non-viral methods such as electroporation.
[xi] modulation of NK cell subset distribution and relative populations, for instance, based on the influence of the invention on NK cell surface receptors (e.g. activating and inhibitory receptors) or NK cell memory. This could offer the benefit of influencing NK cell performance in vivo (e.g. in the tumor microenvironment).
[xii] differentiation of stem cells or progenitor cells into NK cells, which may be made more effective and efficient by the invention—resulting in more effective NK cellular therapies (e.g. more biologically active iPSC-derived NK cells; iPSC is induced Pluripotent Stem Cell).

In addition to the above summarized utility of the invention pertaining specifically to NK cellular immunotherapy, additional utility of the invention may be offered through:
[xiii] enhancement of the biological functions of other cells of the immune system, i.e. cellular immunity displayed by immune cells other than NK cells which can impart cell-mediated immunity e.g. T-cells, monocytes, macrophages, neutrophils, dendritic cells, mast cells, eosinophils, basophils, including subsets and combinations thereof. Also included are specific subsets of immune cells such as Natural Killer T cells (NK-T cells), and gamma-delta(γγ) T cells. A specific example (amongst several possible examples) is enhanced phagocytosis exhibited by macrophages, when cultured in the preferred embodiment of the media formulation described in this invention. Better therapeutic outcomes could be achieved for monotherapy or combination therapy.
[xiv] enhancement of the effectiveness of humoral immunity, e.g. through improved biological functions of B-cells of the immune system that generate antibodies against specific antigens. Better therapeutic outcomes could be achieved for monotherapy or combination therapy.
[xv] use of the invention as a direct therapy, for example if the formulation(s) resulting from the invention were injected directly in vivo (e.g. intratumorally to induce better immune function against cancer), rather than to culture immune cells ex vivo.
[xvi] use of the invention for in vitro diagnostics using immune cells, where the biological functions of the immune cell(s) is/are enhanced, and improved sensitivity is achieved for in vitro diagnostics—for e.g. for the purpose of developing personalized immunotherapy regimen.
[xvii] enhancement of the biological functions of non-immune cells, specific examples include hybridoma cells, CHO cells (CHO: Chinese Hamster Ovary) etc, which can offer the benefit of improved productivity in biomanufacturing macromolecular biotechnology products.

Given the elucidation of the invention above, and as summarized in specific terms of the preferred embodiment corresponding to Table-01, the next section below highlights several working examples of reduction to practice, and description of the utility of the preferred embodiment and other embodiments of the invention, with illustrative experimental data.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1: Enhancement of NK cell Cytotoxicity by Enable-NK media

Enable-NK media enhances the cytotoxicity of Natural Killer (NK) cells against diseased cells such as cancer cells. The enhancement of NK cell cytotoxicity facilitated by Enable-NK media is comprehensive, and covers all three broad-based mechanisms of NK cell cytotoxicity: [a] natural/cellular cytotoxicity, independent of antigen recognition, [b] ADCC (Antibody Dependent Cell-mediated Cytotoxicity) based on antigen recognition, and [c] signaling to other immune cells for amplification of the immune response. Enable-NK media enhances the manifestation of all these three cytotoxicity mechanisms of NK cells, as illustrated by experimental data disclosed herein

Experimental methodology: For the experimental evaluation of the influence of Enable-NK media on Natural Killer (NK) cell cytotoxicity, the methodology centered on the in vitro quantification of the viability of cancer cells when co-cultured in the presence of the NK cells. The ratio of the effector NK cells to the target cancer cells (Effector:Target ratio, i.e. E:T ratio), as well as the duration of the co-culture are examples of experimental factors which directly influence the outcome of such a co-culture assay, with higher E:T ratios and longer durations of co-culture incubation resulting in higher cytotoxicity of the target cancer cells.

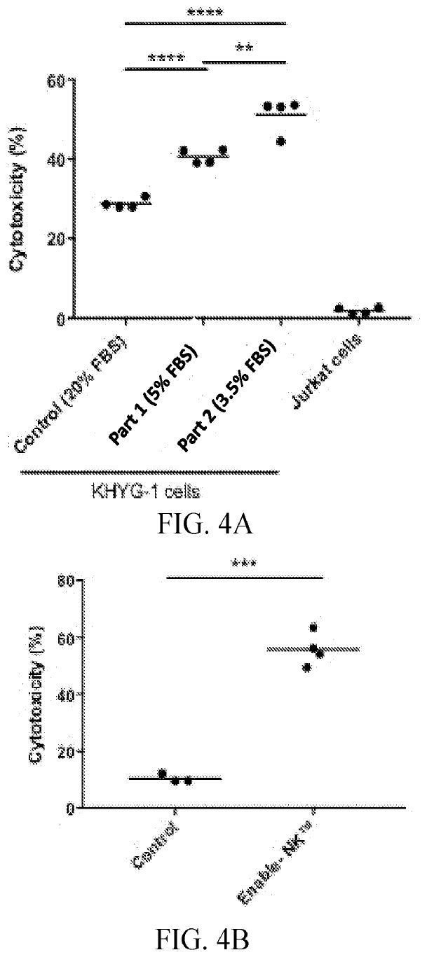

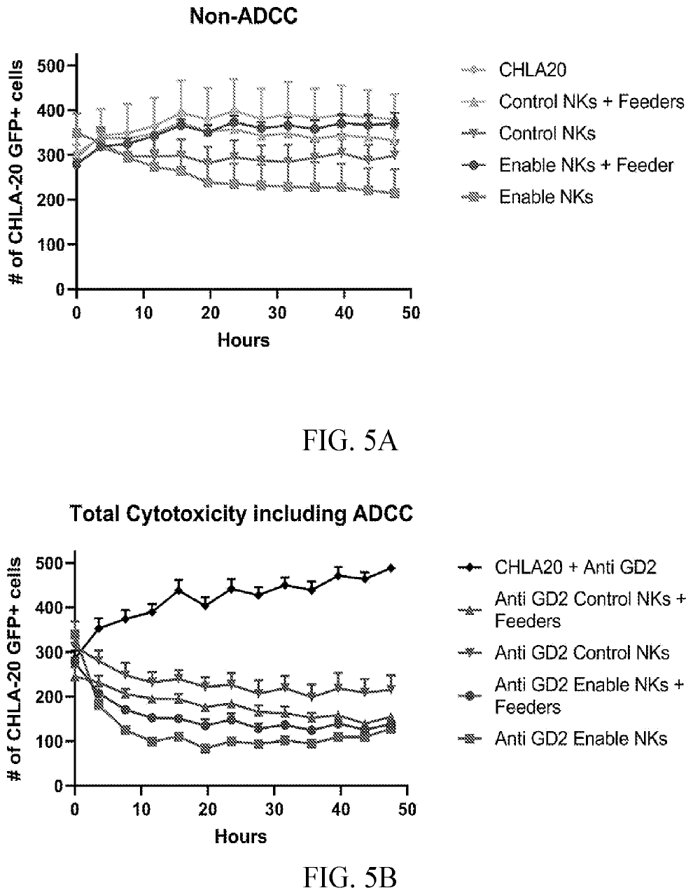

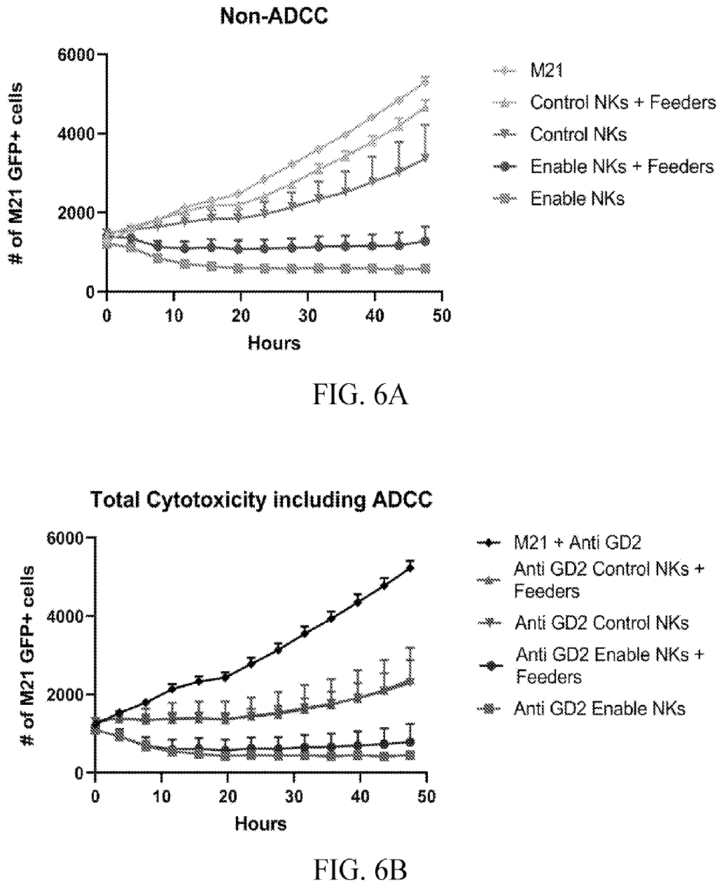

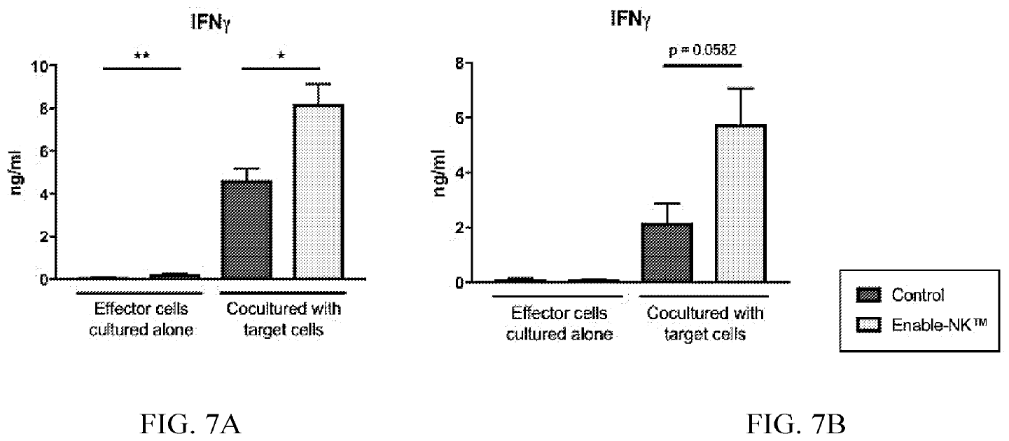

With the above described experimental framework at its core, the experimental determination of the specific influence of Enable-NK media on the cytotoxicity of NK cells was based on the quantification of relative cytotoxicity between experimental groups, where the experimental groups differed only in the media composition for the culturing/expansion of NK cells prior to use of the cultured/expanded NK cells in the above-described co-culture assay with cancer cells. As outlined in FIGS. 4-8, comparisons were made between media compositions—for e.g. DMEM-F12 media (at a higher serum concentration) was compared to DMEM-F12 media combined with Enable-NK ingredients (at a lower serum concentration), with the co-culture assay incubation (of NK effector cells with target cancer cells) occurring in their respective media conditions (FIG. 4, 7, 8). Another example is the comparison between RPMI-1640 media and Enable-NK media for the expansion of NK cells (both at the same serum levels), with the co-culture assay incubation (of NK effector cells with target cancer cells) occurring in identical media conditions (DMEM, which is different from either of the two experimental groups), as illustrated in FIGS. 5 and 6. The NK cells used in these co-culture assays were the human NK cell line KHYG1 (FIG. 4, 7), as well as primary human NK cells from healthy adult donors (FIG. 4-8). The target cancer cells used were K562 leukemia cells (FIGS. 4, 7), CHLA-20 neuroblastoma cells (FIG. 5), and M21 melanoma cells (FIG. 6).

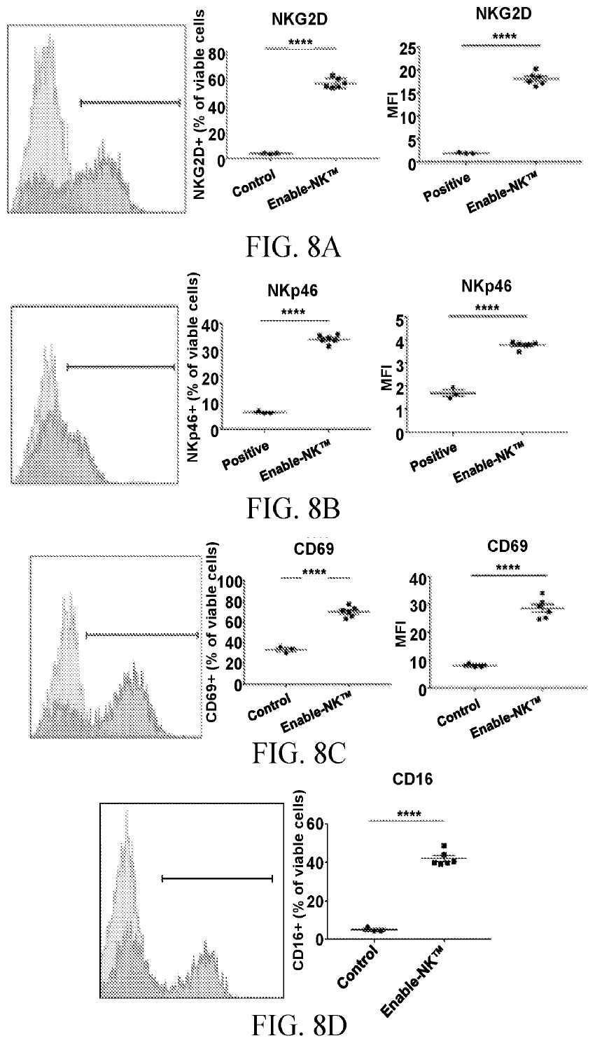

The end-point quantification for target cell viability was pursued with Propidium Iodide (P.I.) staining evaluated through flow cytometry (FIG. 4), as was biomarker quantification (FIG. 8)—where the target cells were differentiated from the effector cells using Carboxyfluorescein succinimidyl ester (CFSE) staining. Target cell viability was also determined by the quantification of Green Fluorescent Protein (GFP) in viable target cancer cells through the Sartorius Incucyte® live-cell analysis system (FIG. 5, 6). Quantification of soluble Interferon-gamma, IFN-γ (FIG. 7) in NK cell supernatant samples was pursued using the Luminex Magpix instrument.

Key results: The media conditions for FIG. 4 were as follows: Control media was DMEM/F12 [at 20% (v/v) fetal bovine serum (FBS) for FIG. 4A, and 10% human AB serum for FIG. 4B]. Enable-NK media for FIG. 4A (KHYG1 NK cells) was DMEM/F12 with the proprietary Enable-NK ingredients [at a final FBS concentration of 5% (v/v) for Part-I alone and 3.5% (v/v) for Part-II media (following Part-I incubation)]. Enable-NK media for FIG. 4B (primary NK cells) was DMEM/F12 with the proprietary Enable-NK ingredients [at a final human AB serum concentration of 2.5% (v/v)]. Co-culture incubation with K562 cells for different experimental groups was set up in their respective media at the same E:T ratio (5:1).

Data summarized in FIG. 5 illustrates that primary NK cell cytotoxicity observed against the target cancer cell line CHLA-20 was greater for Enable-NK media, in direct comparison to Control media (RPMI-1640). This is indicated by the lower values on the y-axis (number of target cells remaining in co-culture) for the Enable-NK media conditions when compared to the corresponding Control media conditions. Greater NK cell cytotoxicity corresponding to Enable-NK media holds true whether or not the expansion of primary NK cells was pursued in the presence of K562 feeder cells, and whether or not the anti-GD2 antibody was used (ADCC).

Data summarized in FIG. 6 illustrates that primary NK cell cytotoxicity observed against the target cancer cell line M21 was greater for Enable-NK media, in direct comparison to Control media (RPMI-1640). This is indicated by the lower values on the y-axis (number of target cells remaining in co-culture) for the Enable-NK media conditions when compared to the corresponding Control media conditions. Greater NK cell cytotoxicity corresponding to Enable-NK media holds true whether or not the expansion of primary NK cells was pursued in the presence of K562 feeder cells, and whether or not the anti-GD2 antibody was used (ADCC).

The media conditions for FIG. 7 were as follows: Control media was DMEM/F12 [at 20% (v/v) fetal bovine serum (FBS) for FIG. 7A, and 10% human AB serum for FIG. 7B]. Enable-NK media for FIG. 7A (KHYG1 NK cells) was DMEM/F12 with the proprietary Enable-NK ingredients [and at a final FBS concentration of 2% (v/v) in Part-II media (following gradual serum reduction in Part-I incubation from 20% down to 2%)]. Enable-NK media for FIG. 4B (primary NK cells) was DMEM/F12 with the proprietary Enable-NK ingredients [and at a final human AB serum concentration of 2.5% (v/v) in Part-II media (following gradual serum reduction in Part-I incubation from 10% down to 2.5%)]. NK cells were expanded for a week in their respective media before the overnight incubation. Corresponding to the primary NK cell data (FIG. 7B, monoculture), several cell-surface biomarkers related to NK cell activation were also quantified by flow cytometry, and these are summarized in FIG. 8 below.

The data summarized in FIG. 8 corresponds to primary NK cells from the same experiment that FIG. 7 refers to, with identical experimental conditions for media composition and NK cell incubation in these media conditions. While NKG2D, NKp46 and CD69 are activation markers on the surface of NK cells, CD16 also corresponds to NK cell cytotoxicity, being central to the mechanism of ADCC (Antibody Dependent Cell-mediated Cytotoxicity).

Discussion, Conclusions, and Implications for NK cellular therapies: As illustrated in FIGS. 4-8, three distinct but complementary aspects of NK cell cytotoxicity are enhanced by Enable-NK media for the KHYG1 NK cell line, as well as primary NK cells, i.e. [i] natural cytotoxicity [ii] Antibody Dependent Cell-mediated Cytotoxicity (ADCC), and [iii] NK signaling to other immune cells (to recruit them and amplify the overall immune response against cancer).

The biological significance of enhanced cytotoxicity of NK cells is enhanced potency of Natural Killer immunotherapies against cancer and other diseases (Myers et al, 2021; Shimasaki et al, 2020). Specifically, there are 3 broad mechanisms of NK cell cytotoxicity directed against diseased cells, which are distinct but complementary i.e. [i] natural/cellular cytotoxicity (Kumar et al, 2022), [ii] ADCC (Antibody Dependent Cell-mediated Cytotoxicity, Ochoa et al, 2017), and [iii] NK cell signaling and recruitment of other immune cells (e.g. T cells) through the secretion of cytokines such as Interferon-gamma (IFN-γ) (Hodgins et al, 2019). Natural cytotoxicity is enhanced when the balance of activating receptors and inhibiting receptors on the surface of NK cells shifts in favor of the activating receptors—data in this section (Example 1) and the next section (Example 2) illustrates this change in balance facilitated by Enable-NK media. Modulation of specific subpopulations of NK cells (Example 2 data) also influences NK cell cytotoxicity. The enhancement of all 3 of these mechanisms of NK cell cytotoxicity by Enable-NK media offers the opportunity for synergy as well as broad clinical applicability of NK cellular therapies against a range of diseases including cancer (Kumar et al, 2022; Nigro et al, 2019).

In summary, the experimental results summarized in this Example provide evidence that the Enable-NK media formulation can support greater effectiveness of NK cellular therapies, by virtue of enhancing the cytotoxicity of NK cells, and NK cell signaling to other immune cells.

### REFERENCES FOR EXAMPLE 1

Hodgins, J. J., Khan, S. T., Park, M. M., Auer, R. C. and Ardolino, M., 2019. Killers 2.0: NK cell therapies at the forefront of cancer control. The Journal of clinical investigation, 129(9), pp. 3499-3510.
Kumar, A., Khani, A. T. and Swaminathan, S., 2022. Harnessing Natural Killer Cell-Mediated Innate Immune Responses for Cancer Treatment: Advances and Challenges. Exploratory Research and Hypothesis in Medicine, (000), pp. 0-0.
Myers, J. A. and Miller, J. S., 2021. Exploring the NK cell platform for cancer immunotherapy. Nature reviews Clinical oncology, 18(2), pp. 85-100.
Nigro, C. L., Macagno, M., Sangiolo, D., Bertolaccini, L., Aglietta, M. and Merlano, M. C., 2019. NK-mediated antibody-dependent cell-mediated cytotoxicity in solid tumors: biological evidence and clinical perspectives. Annals of translational medicine, 7(5).
Ochoa, M. C., Minute, L., Rodriguez, I., Garasa, S., Perez-Ruiz, E., Inogés, S., Melero, I. and Berraondo, P., 2017. Antibody-dependent cell cytotoxicity: immunotherapy strategies enhancing effector NK cells. Immunology and cell biology, 95(4), pp. 347-355.
Shimasaki, N., Jain, A. and Campana, D., 2020. NK cells for cancer immunotherapy. Nature reviews Drug discovery, 19(3), pp. 200-218.

### Example 2: Mitigation of NK cell Exhaustion by Enable-NK media

Enable-NK media mitigates the exhaustion of Natural Killer (NK) cells as well as other mechanisms of NK cell dysfunction i.e. anergy and senescence. The dysfunction of NK cells entails cellular exhaustion and/or anergy and/or senescence as represented by: [a] reduced effector function i.e. reduced cytotoxic activity of NK cells against diseased cells (e.g. cancer cells), and [b] modified expression levels of biomarkers i.e. increase in PD-1, TIM-3, LAG-3, TIGIT, NKG2A, and a decrease in CD16 and CD57. Enable-NK media mitigates the manifestation of these outcomes of NK cell dysfunction, as illustrated by experimental data summarized below.

Experimental Methodology: The experimental methodology was based on running long-term co-cultures of NK cells with target cancer cells in G-Rex® [Gas Permeable Rapid Expansion] plates from Wilson Wolf Manufacturing Corporation in St Paul, MN. Since the membrane-based G-Rex® platform facilitates gentle media change without disturbing cells sitting on top of the membrane, it allowed for the evaluation of the same co-culture samples over a longer period of time.

In separate experiments, both KHYG-1 NK cells and primary NK cells (from 3 healthy adult donors) were cultured in either Control media (DMEM/F12) or Enable-NK media (DMEM/F12+Enable-NK ingredients/additives) prior to the setup of co-culture experimentation with K562-GFP leukemia cells at an Effector: Target (E:T) ratio of 1:1. For the experiment with the KHYG-1 cell line, the culture in Enable-NK media was pursued first in Enable-NK Part-I media (DMEM/F12+Enable-NK Part-I ingredients/additives) for 7 days, followed by Enable-NK Part-II media (DMEM/F12+Enable-NK Part-II ingredients/additives) for 1 day. The 1:1 co-cultures were then established in separate wells of G-Rex 24-well plates (Wilson Wolf, P/N 80192M), with the addition of 3 million effector cells and 3 million target cells into each well on day 0. The fetal bovine serum (FBS) content in Control media was maintained at 20% (v/v), and in Enable-NK Part-I media was gradually reduced from 20% (v/v) down to 5% (v/v), prior to the transition to Enable-NK Part-II media also at 5% FBS content. For the experiment with primary NK cells (from 3 donors), the culture in Enable-NK media was pursued first in Enable-NK Part-I media for 3 days, followed by Enable-NK Part-II media for 1 day. The 1:1 co-cultures were then established in separate wells of G-Rex 24-well plates, with the addition of 2 million effector cells and 2 million target cells into each well on day 0. Serum content was maintained at 10% (v/v) human AB serum for both the Control media (DMEM/F12), as well as Enable-NK media (DMEM/F12+Enable-NK ingredients/additives).

The target K562-GFP leukemia cells were differentiated from the effector NK cells, by virtue of Green Fluorescent Protein (GFP) fluorescence; the K562-GFP cells were procured from American Type Culture Collection (ATCC, catalog #CCL-243-GFP). Propidium Iodide (PI) staining was used for determination of cell death/viability. Fluorescently labeled antibodies against various cell-surface proteins (e.g. PD-1, TIM-3, LAG-3, TIGIT etc.) were used for detection, and flow cytometry analysis was performed (with adequate steps for gating and compensation) at the University of Connecticut Health Flow Cytometry Facility.

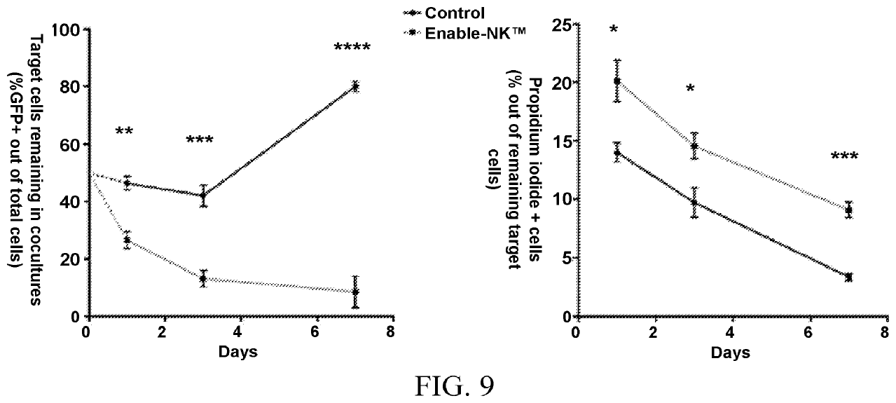

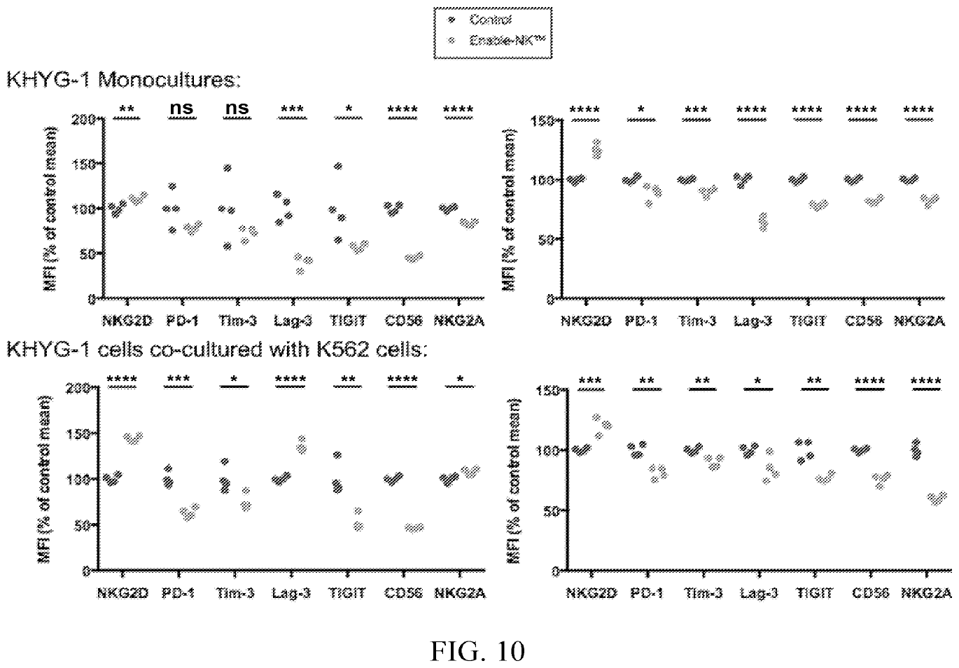

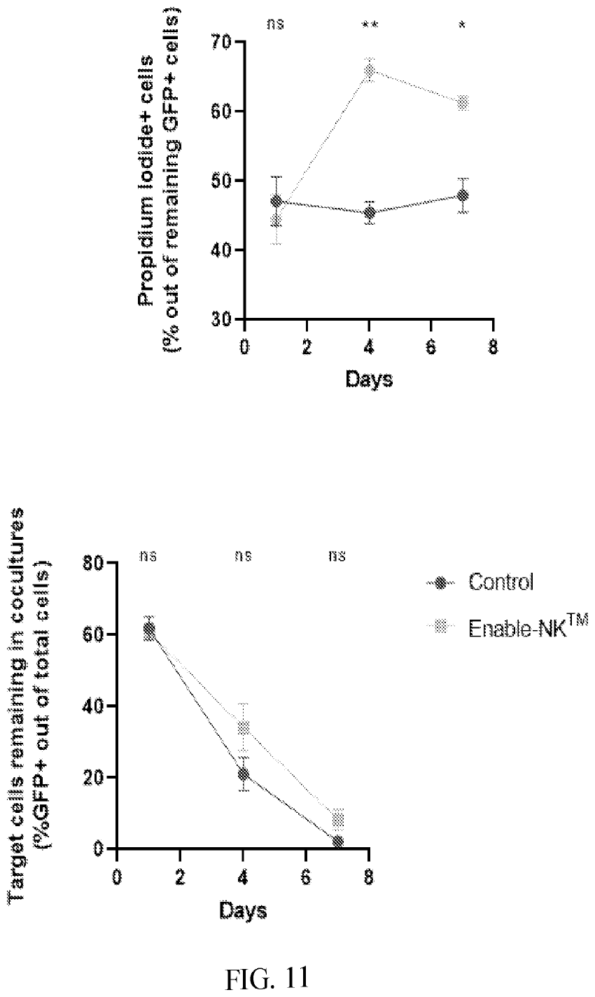

Key Results: The key experimental results are summarized in FIGS. 9 and 10 for the NK cell line, KHYG-1, and in FIGS. 11-16 for experimentation with primary NK cells.

The two media conditions for FIGS. 9 & 10 were as follows: Control media was DMEM/F12 at 20% (v/v) fetal bovine serum (FBS). Enable-NK media was DMEM/F12 with the proprietary Enable-NK additives, and at a final serum concentration of 5% (v/v) fetal bovine serum (FBS).

The two media conditions for FIGS. 11 & 12 were as follows: Control media was DMEM/F12 at 10% (v/v) human AB serum (HS). Enable-NK media was DMEM/F12 with the proprietary Enable-NK additives, and also at 10% (v/v) human AB serum (HS).

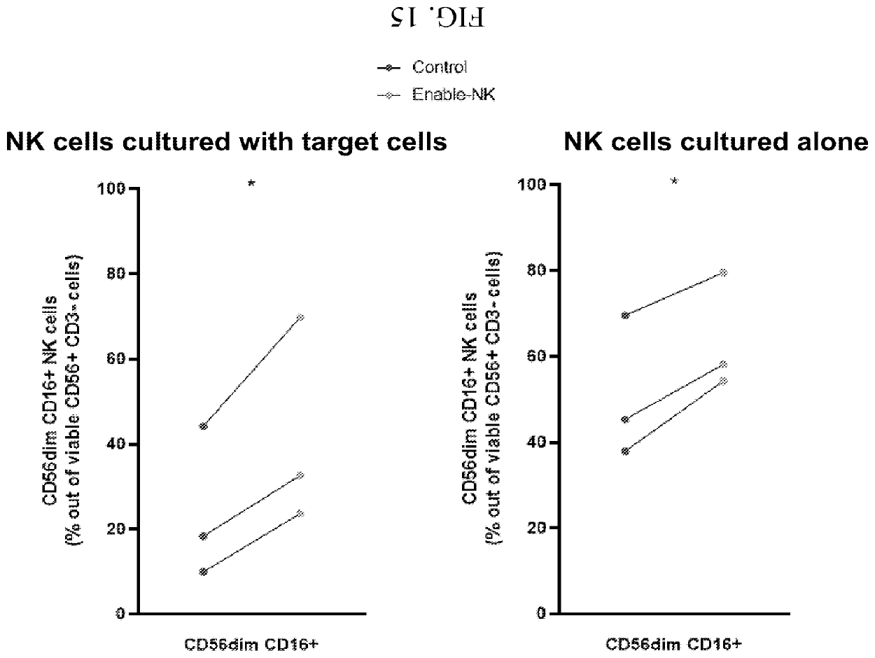

The two media conditions for FIGS. 13 & 14 were as follows: Control media was DMEM/F12 at 10% (v/v) human AB serum (HS). Enable-NK media was DMEM/F12 with the proprietary Enable-NK additives, and also at 10% (v/v) human AB serum (FBS).

The two media conditions for FIGS. 15 & 16 were as follows: Control media was DMEM/F12 at 10% (v/v) human AB serum (HS). Enable-NK media was DMEM/F12 with the proprietary Enable-NK additives, and also at 10% (v/v) human AB serum (FBS).

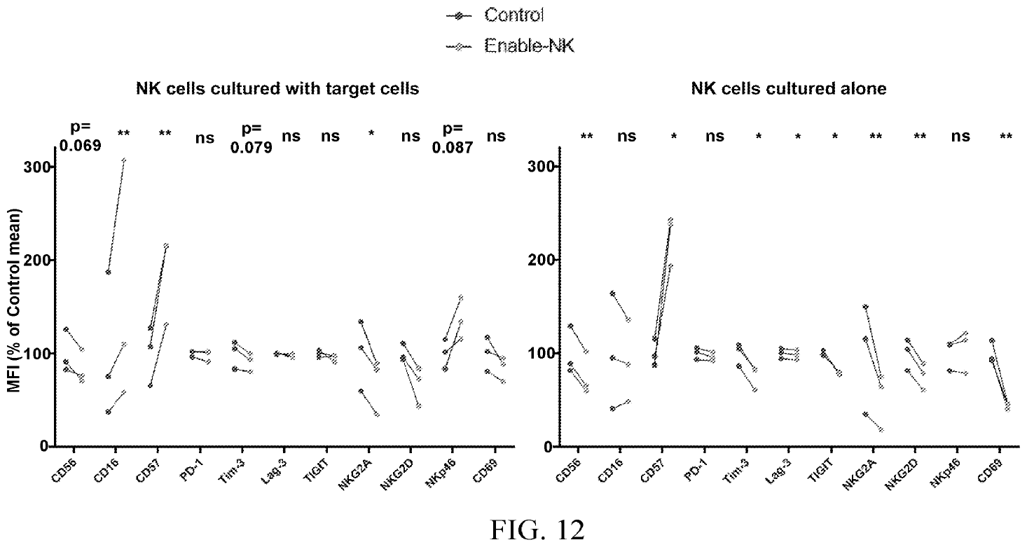

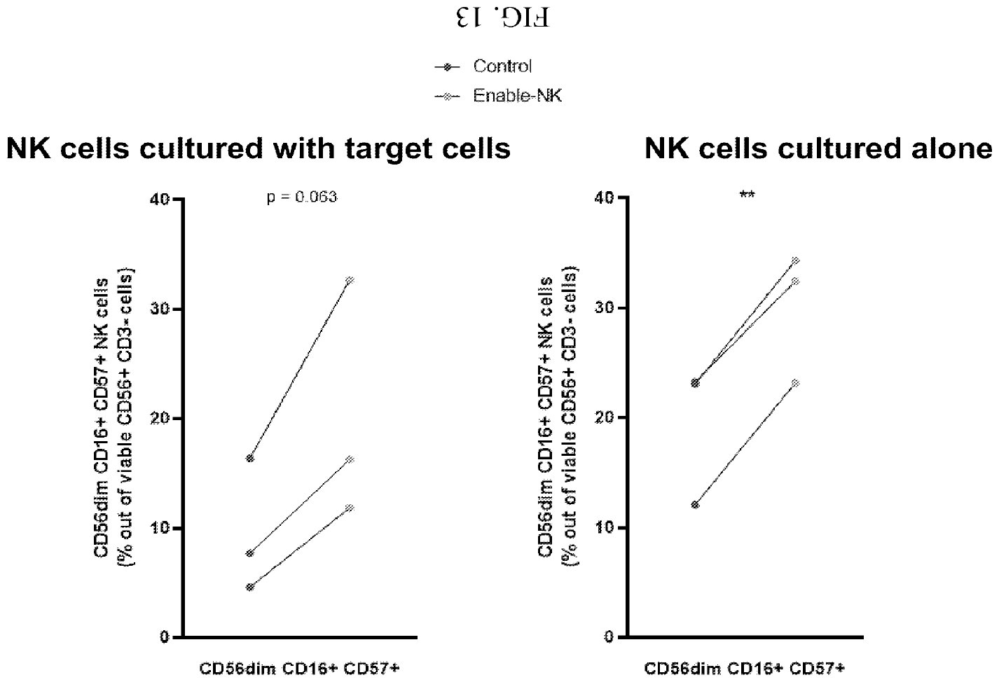

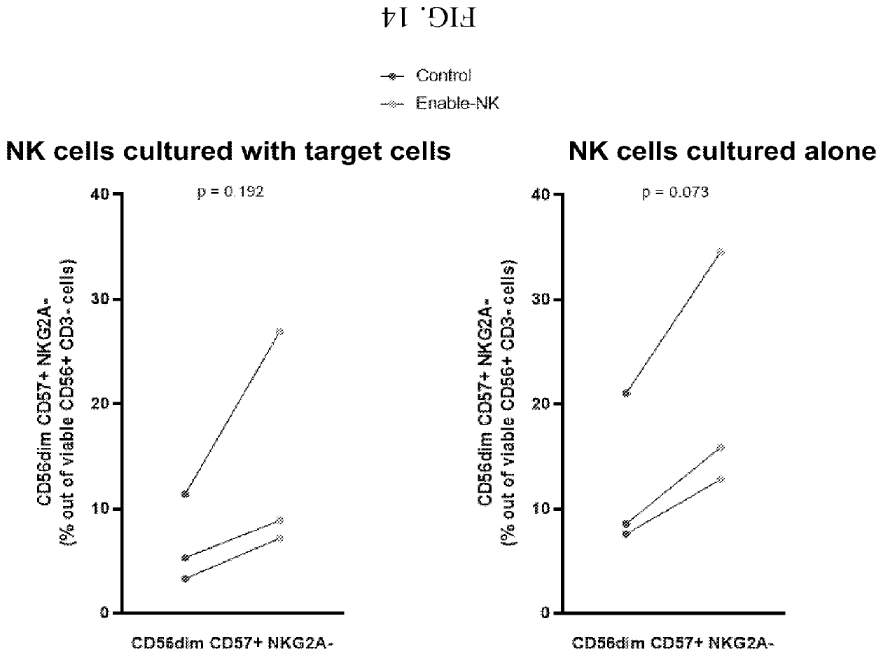

Discussion, Conclusions and Implications for NK cellular therapies: The key end-points representing cellular exhaustion are: [i] diminished cytotoxicity and [ii] increased expression levels of all or some of 5 (five) inhibitory cell-surface biomarkers i.e. PD-1, TIM-3, LAG-3, TIGIT and NKG2A. As illustrated in FIGS. 9-12, both these end-point trends are mitigated by virtue of culturing NK cells in the Enable-NK media, and this mitigation of the NK cell exhaustion phenotype was observed with the NK cell-line KHYG-1 (FIGS. 9-10), as well as primary NK cells (FIGS. 11-12).

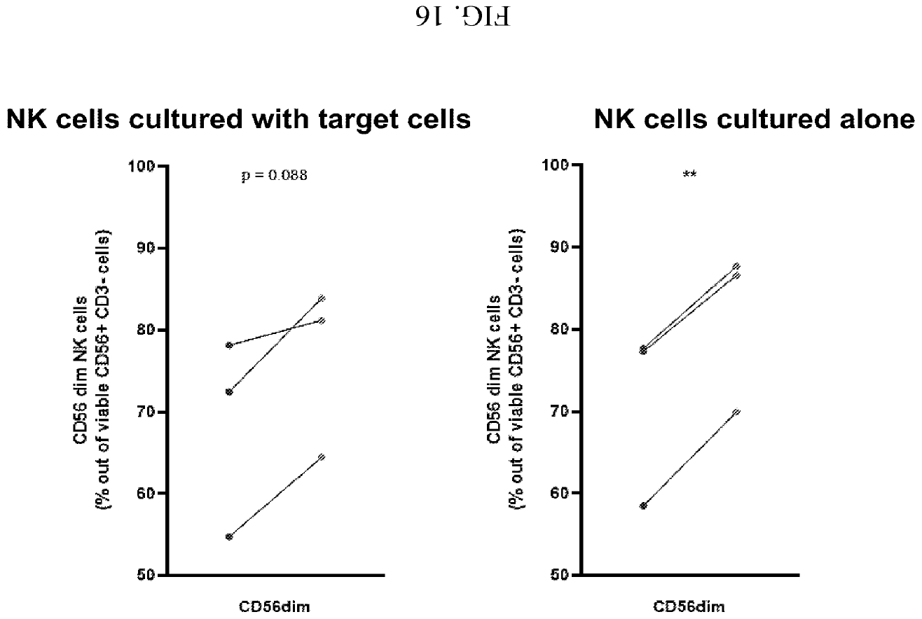

The biological significance of the mitigation of NK cell dysfunction (exhaustion and/or anergy and/or senescence) is increased potential for solid tumor infiltration by NK cells, as well as decreased dysfunction of NK cells in the tumor microenvironment (Judge et al, 2020 and Wang et al, 2020). Based on the data represented in FIGS. 9-14, Enable-NK mitigates NK cell exhaustion (reduced levels of PD-1, TIM-3, LAG-3, and TIGIT), it mitigates NK cell anergy (higher levels of CD16 expression), and also mitigates NK cell senescence (reduced NKG2A expression and increased CD57 expression). Additionally, the biological significance of higher levels of the CD56^{dim} sub-population (FIG. 10, 12, 16), CD56_{dim}CD16+CD57⁺ sub-population (FIG. 13), CD56^{dim}CD57⁺NKG2A⁻ sub-population (FIG. 14) and CD56_{dim}CD16+ (FIG. 15) sub-population supported by Enable-NK is that of higher cytotoxicity levels represented by these sub-populations of NK cells (Lopez-Verges et al, 2010; Beziat et al, 2010, Haanen et al, 2018).

In summary, the experimental results summarized in this Example provide evidence that the Enable-NK media formulation can support greater effectiveness of NK cellular therapies, by virtue of the mitigation of NK cell dysfunction represented by exhaustion and/or anergy and/or senescence.

### REFERENCES FOR EXAMPLE 2

Béziat, V., Descours, B., Parizot, C., Debré, P. and Vieillard, V., 2010. NK cell terminal differentiation: correlated stepwise decrease of NKG2A and acquisition of KIRs. PloS one, 5(8), p.e11966.
Haanen, J. B. and Cerundolo, V., 2018. NKG2A, a new kid on the immune checkpoint block. Cell, 175(7), pp. 1720-1722.
Judge, S. J., Murphy, W. J. and Canter, R. J., 2020. Characterizing the dysfunctional NK cell: assessing the clinical relevance of exhaustion, anergy, and senescence. Frontiers in cellular and infection microbiology, 10, p.49.
Lopez-Vergés, S., Milush, J. M., Pandey, S., York, V. A., Arakawa-Hoyt, J., Pircher, H., Norris, P. J., Nixon, D. F. and Lanier, L. L., 2010. CD57 defines a functionally distinct population of mature NK cells in the human CD56dimCD16⁺ NK-cell subset. Blood, The Journal of the American Society of Hematology, 116(19), pp. 3865-3874.
Wang, J. and Matosevic, S., 2020. Functional and metabolic targeting of natural killer cells to solid tumors. Cellular Oncology, 43(4), pp. 577-600.

### Example 3: Enhancement of NK Cell Metabolism by Enable-NK Media

Enable-NK media enhances the metabolism of Natural Killer (NK) cells, and this effect is consistent with its mitigation of NK cell exhaustion (covered in Example 2). The metabolic activity of NK cells is represented by the levels of intracellular NAD (Nicotinamide Adenine Dinucleotide). Metabolic activity of NK cells, as represented by NAD levels, is enhanced by Enable-NK media, as illustrated by experimental data summarized below.

Experimental Methodology: KHYG-1 NK cells were cultured in either Control media (DMEM/F12) or Enable-NK media (DMEM/F12+Enable-NK ingredients/additives). The culturing of NK cells in the Enable-NK media was pursued first in Enable-NK Part-I media (DMEM/F12+Enable-NK Part-I ingredients/additives) for 3 days, followed by Enable-NK Part-II media (DMEM/F12+Enable-NK Part-II ingredients/additives) for another 4 days (total incubation time of one week). For the experiment with KHYG-1 NK cells, the fetal bovine serum (FBS) content in Control media was maintained at 20% (v/v), and in Enable-NK Part-I media, was gradually reduced from 20% (v/v) down to 5% (v/v), prior to the transition to Enable-NK Part-II media also at 5% FBS content.

The method of detection for NAD (Nicotinamide Adenine Dinucleotide) was through the total quantification of both the oxidized and reduced forms of NAD (NAD+/NADH), as measured by a bioluminescence based kit from Promega Corporation (Madison WI, Catalog #G9071). The total intracellular NAD was normalized to that observed in the Control media (NT, i.e. No Treatment).

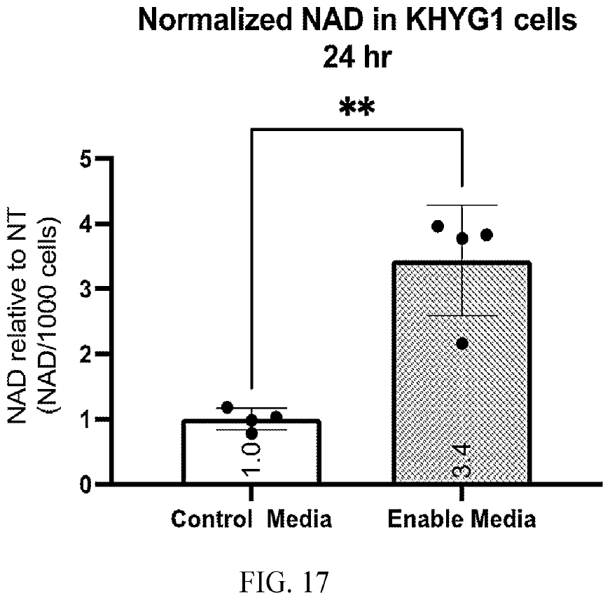

Results: The levels of intracellular NAD (Nicotinamide Adenine Dinucleotide) i.e. NAD values normalized to Control media are summarized in FIG. 17 for the two media conditions, with more than a 3× difference between the two media conditions.

The two media conditions for FIG. 17 were as follows: Control media was DMEM/F12 at 20% (v/v) fetal bovine serum (FBS). Enable-NK media was DMEM/F12 with the proprietary Enable-NK additives, and at a final serum concentration of 5% (v/v) fetal bovine serum (FBS).

Discussion, Conclusions and Implications for NK cellular therapies: The normalized NAD measurements in this study are an indicator of the metabolic robustness of the NK cells. Nicotinamide adenine dinucleotide (NAD) is a coenzyme involved in the regulation of various metabolic pathways, including glycolysis, p-oxidation, and oxidative phosphorylation. NAD also functions as a substrate for poly(ADP-ribose) polymerase (PARP), sirtuin, and NAD glycohydrolase. Moreover, NAD regulates DNA repair, gene expression, energy metabolism, and stress responses (Yaku et al, 2019). In general, Nicotinamide Adenine Dinucleotide (NAD) and its metabolome (NADome) play an important role in maintaining cellular homeostasis (Braidy et al, 2021). The relevance of NAD to the immunometabolism of NK cells is well established, for example in the context of energy metabolism in general and mitochondrial metabolism in particular (O'Brien et al, 201, Choi et al, 2021, and Stein et al, 2012)—providing the basis for the therapeutic potential of NAD metabolism (Xie et al, 2020, Takeda et al, 2021).

The biological significance of enhanced metabolism of NK cells is enhanced potency of Natural Killer immunotherapies against solid tumors, given the negative impacts of the tumor microenvironment (TME) on NK cell metabolism (Terren et al, 2019). Because the activation of NK cells for antitumor effector functions is manifested as enhanced metabolic activity such as glycolysis and the upregulation of nutrient transporters, therefore enhancement of the immunometabolism of NK cells in pathophysiologic conditions can enhance the clinical effectiveness of NK cellular immunotherapies (Wang et al, 2020, Terren et al, 2020).

In summary, the experimental results summarized in this Section C provide evidence that the Enable-NK media formulation can support greater effectiveness of NK cellular therapies, by virtue of enhanced metabolism of NK cells.

### REFERENCES FOR EXAMPLE 3

Braidy, N., Villalva, M. D. and Grant, R., 2021. NADomics: Measuring NAD+ and Related Metabolites Using Liquid Chromatography Mass Spectrometry. Life, 11(6), p.512.
Choi, C. and Finlay, D. K., 2021. Optimising NK cell metabolism to increase the efficacy of cancer immunotherapy. Stem Cell Research & Therapy, 12(1), pp. 1-10.
O'Brien, K. L. and Finlay, D. K., 2019. Immunometabolism and natural killer cell responses. Nature Reviews Immunology, 19(5), pp. 282-290.
Stein, L. R. and Imai, S. I., 2012. The dynamic regulation of NAD metabolism in mitochondria. Trends in Endocrinology & Metabolism, 23(9), pp. 420-428.
Takeda, K. and Okumura, K., 2021. Nicotinamide mononucleotide augments the cytotoxic activity of natural killer cells in young and elderly mice. Biomedical Research, 42(5), pp. 173-179.
Terrén, I., Orrantia, A., Vitallé, J., Zenarruzabeitia, O. and Borrego, F., 2019. NK cell metabolism and tumor microenvironment. Frontiers in Immunology, 10, p.2278.
Terrén, I., Orrantia, A., Vitallé, J., Astarloa-Pando, G., Zenarruzabeitia, O. and Borrego, F., 2020, October. Modulating NK cell metabolism for cancer immunotherapy. In Seminars in Hematology (Vol. 57, No. 4, pp. 213-224). WB Saunders.
Wang, J. and Matosevic, S., 2020. Functional and metabolic targeting of natural killer cells to solid tumors. Cellular Oncology, 43(4), pp. 577-600.
Xie, N., Zhang, L., Gao, W., Huang, C., Huber, P. E., Zhou, X., Li, C., Shen, G. and Zou, B., 2020. NAD+ metabolism: pathophysiologic mechanisms and therapeutic potential. Signal transduction and targeted therapy, 5(1), pp. 1-37.
Yaku, K., Okabe, K., Gulshan, M., Takatsu, K., Okamoto, H. and Nakagawa, T., 2019. Metabolism and biochemical properties of nicotinamide adenine dinucleotide (NAD) analogs, nicotinamide guanine dinucleotide (NGD) and nicotinamide hypoxanthine dinucleotide (NHD). Scientific reports, 9(1), pp. 1-12.

### Example 4: Enhancement of NK cell Viral Transduction by Enable-NK Media

Enable-NK enhances the efficiency of viral transduction of Natural Killer (NK) cells, exemplified by the retroviral transduction of the NK cell line NK-92, and as illustrated by the experimental data summarized below.

Experimental methodology: Viral transduction of NKp44-FLAG constructs into NK-92 cells was pursued via a bicistronic retroviral vector containing an internal ribosome entry site (IRES) [NKp44 tagged with FLAG (extracellular end) was put into the pBMN-IRES-GFP vector (with the GFP edited out), Phoenix-Ampho viral packaging cell line]. NK-92 cells were cultured in α-MEM medium or Enable-NK Part-II medium for 2 weeks, prior to transduction, then cultured for another 6 days in the two media. Both media conditions had 100 IU/ml IL-2. α-MEM medium had 10% (v/v) heat-inactivated FBS and 10% (v/v) horse serum, while the Enable-NK Phase-II medium had 5% (v/v) heat-inactivated FBS. Cells were then stained for both NKp44 and FLAG, and analyzed (flow cytometry) for determining transduction efficiency (percent double-stained cells for both NKp44 and FLAG out of the total NK cell population).

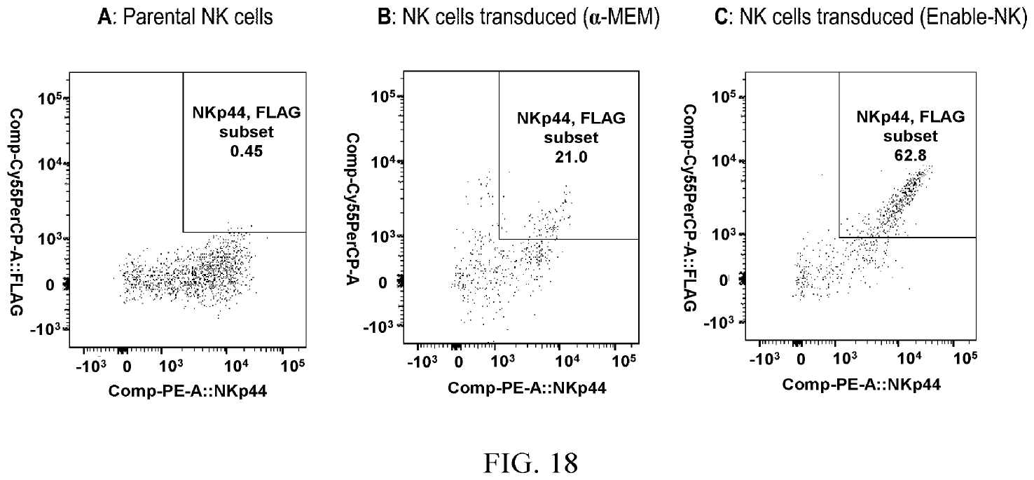

Results: FIG. 18 demonstrates a comparison of the efficiency of transduction on NK-92 cells in α-MEM medium (21.0%) compared to Enable-NK Part-II medium (62.8%), as confirmed by the double-staining of both NKp-44 and FLAG. In contrast, the parental cells displayed no significant staining (<1%).

Discussion, Conclusions and Implications for NK cellular therapies: Significantly higher transduction efficiency (62.8%) was accomplished for retroviral transduction of NK-92 cells with Enable-NK Part-II media, compared to alpha-MEM media (21.0%).

The biological significance of enhanced viral transduction of NK cells is the opportunity for enhanced potency of Natural Killer immunotherapies through the genetic manipulation of NK cells (Childs et al, 2015; Matosevic, 2018). Examples of specific genetic manipulation strategies for NK cells include: [a] better autocrine cytokine stimulation of NK cells by introduction of genes (e.g. IL-2, IL-15, which can obviate dependence on exogenous cytokines) [b] better tumor targeting induced by gene-silencing of inhibitory NK cell receptors (e.g. NKG2A), and [c] improved tumor targeting against specific antigens by the genetic engineering of CARs (Chimeric Antigen Receptors) on the surface of NK cells (Carlsten et al, 2015; Xie et al, 2020). Compared to T-cells, it is more challenging to achieve the viral transduction of Natural Killer (NK) cells, for the purpose of their genetic manipulation (Allan et al, 2021; Carlsten et al, 2016). Specific examples include the low efficiency of viral transduction as well as reduced viability of virally-transduced NK cells, which are hurdles to generating the next generation NK cellular immunotherapies. Therefore, improvements in the efficiency and effectiveness of the viral transduction of NK cells can lead to a wider range of NK cellular therapies, as well as improved effectiveness of NK cellular therapies.

In summary, the experimental results summarized in this Example provide evidence that the Enable-NK media formulation can support greater effectiveness of NK cellular therapies, by virtue of enhanced efficiency of viral transduction of NK cells

### References for Example 4

Allan, D. S., Chakraborty, M., Waller, G. C., Hochman, M. J., Poolcharoen, A., Reger, R. N. and Childs, R. W., 2021. Systematic improvements in lentiviral transduction of primary human natural killer cells undergoing ex vivo expansion. Molecular Therapy-Methods & Clinical Development, 20, pp. 559-571.

Carlsten, M. and Childs, R. W., 2015. Genetic manipulation of NK cells for cancer immunotherapy: techniques and clinical implications. Frontiers in immunology, 6, p.266.

Carlsten, M., Levy, E., Karambelkar, A., Li, L., Reger, R., Berg, M., Peshwa, M. V. and Childs, R. W., 2016. Efficient mRNA-based genetic engineering of human NK cells with high-affinity CD16 and CCR7 augments rituximab-induced ADCC against lymphoma and targets NK cell migration toward the lymph node-associated chemokine CCL19. Frontiers in immunology, 7, p.105.

Childs, R. W. and Carlsten, M., 2015. Therapeutic approaches to enhance natural killer cell cytotoxicity against cancer: the force awakens. Nature reviews Drug discovery, 14(7), pp. 487-498.

Matosevic, S., 2018. Viral and nonviral engineering of natural killer cells as emerging adoptive cancer immunotherapies. Journal of immunology research, 2018.

Xie, G., Dong, H., Liang, Y., Ham, J. D., Rizwan, R. and Chen, J., 2020. CAR-NK cells: A promising cellular immunotherapy for cancer. EBioMedicine, 59, p.102975.

### Example 5: improved Cryopreservation of NK cells by Enable-NK media

Enable-NK improves the cryopreservation of Natural Killer (NK) cells, as illustrated by the experimental data summarized below for primary NK cell cryopreservation performance.

Experimental methodology: Primary NK cells were isolated from a healthy adult human donor, expanded in either Control medium [RPMI-1640, with 1:1 K562 feeder cells (with membrane bound IL-15 and 4-1 BBL)] or Enable-NK Phase-I media (no feeder cells) for a period of twoone weeks with fresh media added every 2-3 days (switching between Enable-NK Phase-I and Phase-II media on day-12). Both media conditions had 10% v/v human AB serumFBS, and 100 IU/ml IL-2 (and 10 ng/ml IL-15 for Enable-NK media). Expanded NK cells (magnetically separated after Control media expansion with feeder cells) were frozen in their respective freezing media. NK cells expanded in the Control medium were frozen in the freezing media recipe of [10% v/v DMSO+45% v/v FBS+45% v/v Control media], and NK cells expanded in the Enable-NK Part-I medium were frozen in the freezing media recipe of [10% v/v DMSO+45% v/v FBS+45% v/v Enable Part-I media]. NK cells frozen in the freezing media recipe of [10% v/v DMSO+45% v/v FBS+45% v/v Control media] were thawed overnight into Control media (no feeder cells), and those frozen in the freezing media recipe of [10% v/v DMSO+45% v/v FBS+45% v/v Enable Part-I media] were thawed overnight in Enable Part-II media (no feeder cells). 10 IU/ml IL-2 was added to the overnight cultures. The next day, NK effector cells from both experimental groups were co-cultured with the human pediatric neuroblastoma cell line, CHLA-20-GFP target cells at an Effector: Target ratio (E:T ratio of 2.5:1). These co-culture studies were pursued under identical DMEM media conditions (w 10% v/v FBS, 100 IU/ml IL-2), with and without the anti-disialoganglioside (anti-GD2) antibody, for cytotoxicity analysis via the Sartorius Incucyte® live-cell analysis instrument.

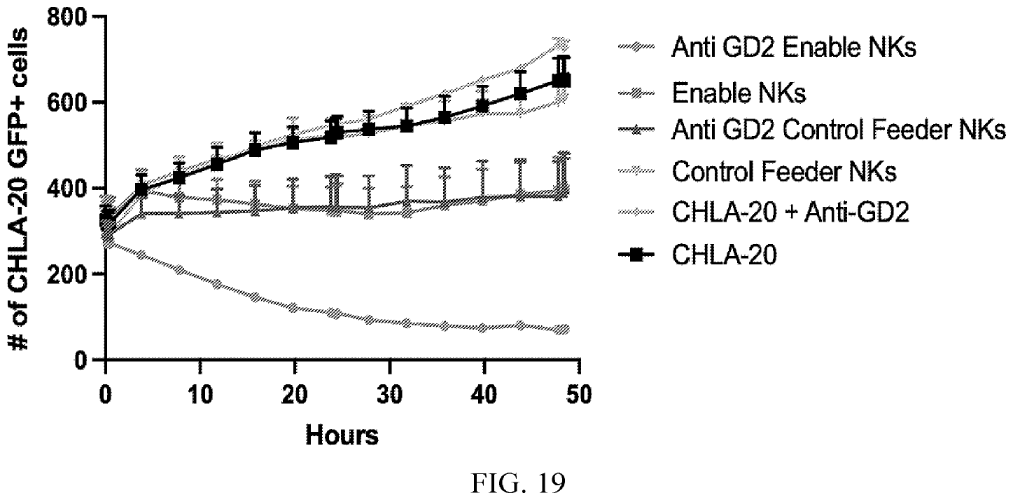

Results: Data procured from the Sartorius Incucyte® instrument is summarized in FIG. 19.

Discussion, Conclusions and Implications for NK cellular therapies: Significantly higher cryopreservation Performance was demonstrated by Enable-NK™ media compared to Control Media, both without the anti-GD2 antibody (natural cytotoxicity), as well as in presence of the anti-GD2 antibody (natural+Antibody Dependent Cell-mediated Cytotoxicity, ADCC). The biological significance of improved cryopreservation of NK cells is the opportunity for better performing off-the-shelf NK cellular therapies for clinical use (Fang et al, 2022). It has been well documented in the published literature that based on in vitro (Mark et al, 2020; Damodharan et al, 2020; Min et al, 2018), in vivo (Miller et al, 2014) as well as clinical studies (Szmania et al, 2015), that cryopreservation (i.e. freezing) and subsequent thawing of NK cells negatively impacts their key functions (e.g. cytotoxicity) and viability, much more so than the cryopreservation of T cells (Fang et al, 2019). These considerations, along with the much shorter half-lives of NK cells, represent significant limitations on the logistics related to the clinical use of NK cellular therapies. There is consensus in the NK research community that continued improvements pertaining to the cryopreservation of NK cells are critical for NK cellular therapeutics to realize their full clinical potential (Kundu et al, 2021).

In summary, the experimental results summarized in this Section E provide evidence that the Enable-NK media formulation can support greater effectiveness of NK cellular therapies, by virtue of improved cryopreservation of NK cells.

### REFERENCES FOR EXAMPLE 5

Damodharan, S. N., Walker, K. L., Forsberg, M. H., McDowell, K. A., Bouchlaka, M. N., Drier, D. A., Sondel, P. M., DeSantes, K. B. and Capitini, C. M., 2020. Analysis of ex vivo expanded and activated clinical-grade human NK cells after cryopreservation. Cytotherapy, 22(8), pp. 450-457.
Fang, F., Wang, W., Chen, M., Tian, Z. and Xiao, W., 2019. Technical advances in NK cell-based cellular immunotherapy. Cancer Biology & Medicine, 16(4), p.647.
Fang, F., Xie, S., Chen, M., Li, Y., Yue, J., Ma, J., Shu, X., He, Y., Xiao, W. and Tian, Z., 2022. Advances in NK cell production. Cellular & Molecular Immunology, 19(4), pp. 460-481.
Kundu, S., Gurney, M. and O'Dwyer, M., 2021. Generating natural killer cells for adoptive transfer: expanding horizons. Cytotherapy, 23(7), pp. 559-566.
Mark, C., Czerwinski, T., Roessner, S., Mainka, A., Hörsch, F., Heublein, L., Winterl, A., Sanokowski, S., Richter, S., Bauer, N. and Angelini, T. E., 2020. Cryopreservation impairs 3-D migration and cytotoxicity of natural killer cells. Nature communications, 11(1), pp. 1-8.
Miller, J. S., Rooney, C. M., Curtsinger, J., McElmurry, R., McCullar, V., Verneris, M. R., Lapteva, N., McKenna, D., Wagner, J. E., Blazar, B. R. and Tolar, J., 2014. Expansion and homing of adoptively transferred human natural killer cells in immunodeficient mice varies with product preparation and in vivo cytokine administration: implications for clinical therapy. Biology of Blood and Marrow Transplantation, 20(8), pp. 1252-1257.
Min, B., Choi, H., Her, J. H., Jung, M. Y., Kim, H. J., Jung, M. Y., Lee, E. K., Cho, S. Y., Hwang, Y. K. and Shin, E. C., 2018. Optimization of large-scale expansion and cryopreservation of human natural killer cells for anti-tumor therapy. Immune network, 18(4).
Szmania, S., Lapteva, N., Garg, T., Greenway, A., Lingo, J., Nair, B., Stone, K., Woods, E., Khan, J., Stivers, J. and Panozzo, S., 2015. Ex vivo expanded natural killer cells demonstrate robust proliferation in vivo in high-risk relapsed multiple myeloma patients. Journal of immunotherapy (Hagerstown, Md.: 1997), 38(1), p.24.

### Example 6: Mitigation of Serum Dependence and Feeder-Cell Use for NK Cell Culturing and Expansion by Enable-NK Media

Enable-NK media supports the culturing and expansion of Natural Killer (NK) cells in a manner that: [a] animal serum dependence is minimized, and [b] the need for feeder cells is minimized or obviated. These benefits for the culturing and expansion of Natural Killer (NK) cells are illustrated by the ex vivo expansion of the human NK cell line KHYG1, as well as primary human NK cells (from healthy adult donors) using Enable-NK media, as summarized by the experimental results delineated below.

Experimental methodology: NK cells lines NK3.3 and KHYG1, as well as primary NK cells from a healthy adult donor were cultured in media conditions summarized below. NK cells were counted using a hemocytometer or a Countess-II instrument (Thermo Fisher, Waltham MA).

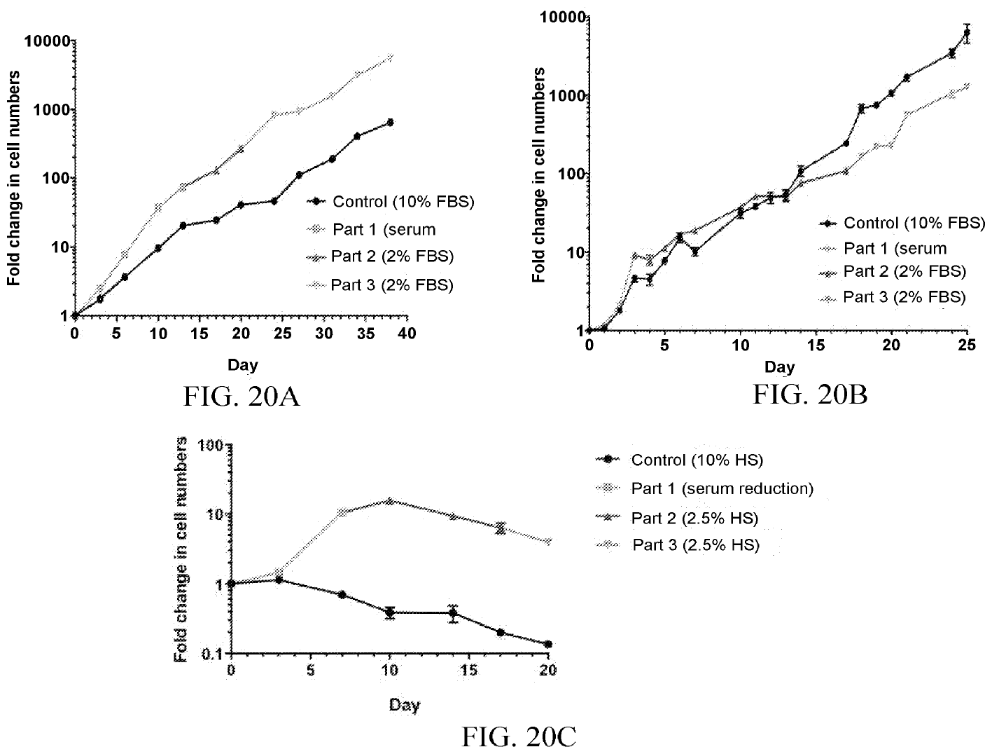

Results: Some key NK cell proliferation results are displayed in FIG. 20. For FIG. 20A, Control media was a standard media [at 10% (v/v) human AB serum], and Enable-NK media was DMEM/F12 with the proprietary Enable-NK additives at reduced serum levels as outlined in FIG. 20A—both with 50 IU/ml IL-2. For FIGS. 20B and 20C, Control media was DMEM/F12 [at 20% (v/v) fetal bovine serum (FBS) for FIG. 20B and at 10% (v/v) human AB serum for FIG. 20C], and Enable-NK media was DMEM/F12 with the proprietary Enable-NK additives at reduced serum levels as outlined in FIGS. 20B and 20C—both with 100 IU/ml IL-2.

Discussion, Conclusions and Implications for NK cellular therapies: Cumulatively, the experimental results for this Example demonstrate that NK cell culturing and expansion is possible in Enable-NK media with significantly reduced serum, and in the absence of feeder cells.

The biological significance of mitigating serum dependence in the culturing and expansion of Natural Killer (NK) cells is the opportunity to avoid the challenges presented by serum content, such as batch-to-batch variability, cost, and risk of pathogen contamination (Karnieli et al, 2017). Due to these considerations, mitigation of serum-dependence is a key process development goal of cellular therapies in general (Campbell et al, 2015) and NK cellular therapies in particular (Moseman et al, 2020). In a similar context, for ex vivo culturing and expansion of Natural Killer (NK) cells, while the use of feeder cells facilitates the achievement of large fold expansion following proliferation of NK cells (Gurney et al, 2022), the use of feeder cells does introduce disadvantages such as suboptimal cytotoxic performance of expanded NK cells (Childs et al, 2013) and possible contamination of the resulting NK cellular therapies with live feeder cells (Kundu et al, 2021).

In summary, the experimental results summarized in this Example provide evidence that the Enable-NK media formulation can support greater effectiveness of NK cellular therapies, by virtue of the culturing and expansion of NK cells with reduced serum or in the absence of serum. These results also provide evidence that the Enable-NK media formulation can support greater effectiveness of NK cellular therapies, by virtue of the culturing and expansion of NK cells in the absence of feeder cells, or with reduced feeder cells. The opportunity to culture and expand NK cells with reduced/absent serum as well as reduced/absent feeder cells i.e. a combined synergistic benefit is also offered by Enable-NK media.

### REFERENCES FOR EXAMPLE 6

Campbell, A., Brieva, T., Raviv, L., Rowley, J., Niss, K., Brandwein, H., Oh, S. and Karnieli, O., 2015. Concise review: process development considerations for cell therapy. Stem cells translational medicine, 4(10), pp. 1155-1163.
Childs, R. W. and Berg, M., 2013. Bringing natural killer cells to the clinic: ex vivo manipulation. Hematology 2013, the American Society of Hematology Education Program Book, 2013(1), pp. 234-246.
Gurney, M., Kundu, S., Pandey, S. and O'Dwyer, M., 2022. Feeder Cells at the Interface of Natural Killer Cell Activation, Expansion and Gene Editing. Frontiers in Immunology, 13.
Karnieli, O., Friedner, O. M., Allickson, J. G., Zhang, N., Jung, S., Fiorentini, D., Abraham, E., Eaker, S. S., Chan, A., Griffiths, S. and Wehn, A. K., 2017. A consensus introduction to serum replacements and serum-free media for cellular therapies. Cytotherapy, 19(2), pp. 155-169.
Kundu, S., Gurney, M. and O'Dwyer, M., 2021. Generating natural killer cells for adoptive transfer: expanding horizons. Cytotherapy, 23(7), pp. 559-566.
Moseman, J. E., Foltz, J. A., Sorathia, K., Heipertz, E. L. and Lee, D. A., 2020. Evaluation of serum-free media formulations in feeder cell-stimulated expansion of natural killer cells. Cytotherapy, 22(6), pp. 322-328.

### ENUMERATED EMBODIMENTS

The following enumerated embodiments are provided, the numbering of which is not to be construed as designating levels of importance.
Embodiment 1 provides a two-part media formulation for culturing Natural Killer (NK) cells comprising:
   a. a first media formulation comprising at least six of following components:
      i. Glycine at a concentration between 3.50E−03 mM and 3.50E+02 mM;
      ii. L-Alanine at a concentration between 1.10E−02 mM and 1.10E+03 mM;
      iii. L-Asparagine at a concentration between 1.50E−03 mM and 1.50E+02 mM;
      iv. L-Aspartic acid at a concentration between 1.50E−03 mM and 1.50E+02 mM;
      v. L-Glutamic Acid at a concentration between 1.50E−03 mM and 1.50E+02 mM;
      vi. L-Proline at a concentration between 2.50E−03 mM and 2.50E+02 mM;
      vii. L-Serine at a concentration between 3.50E−03 mM and 3.50E+02 mM;
      viii. L-Arginine HCl at a concentration between 1.30E−02 mM and 1.30E+03 mM;
      ix. L-Cystine at a concentration between 2.00E−03 mM and 2.00E+02 mM;
      x. L-Histidine HCl—H₂O at a concentration between 3.50E−03 mM and 3.50E+02 mM;
      xi. L-Isoleucine at a concentration between 8.20E−03 mM and 8.20E+02 mM;
      xii. L-Leucine at a concentration between 8.50E−03 mM and 8.50E+02 mM;
      xiii. L-Lysine hydrochloride at a concentration between 8.96E−03 mM and 8.96E+02 mM;
      xiv. L-Methionine at a concentration between 2.21E−03 mM and 2.21E+02 mM;
      xv. L-Phenylalanine at a concentration between 4.20E−03 mM and 4.20E+02 mM;
      xvi. L-Threonine at a concentration between 8.50E−03 mM and 8.50E+02 mM;
      xvii. L-Tryptophan at a concentration between 9.00E−04 mM and 9.00E+01 mM;
      xviii. L-Tyrosine at a concentration between 4.09E−03 mM and 4.09E+02 mM;
      xix. L-Valine at a concentration between 8.50E−03 mM and 8.50E+02 mM;
      xx. Sodium Pyruvate at a concentration between 1.00E−02 mM and 1.00E+03 mM;
      xxi. Arachidonic acid at a concentration between 3.28E−07 mM and 3.28E−02 mM;
      xxii. Linoleic acid at a concentration between 3.78E−06 mM and 3.78E−01 mM;
      xxiii. Linolenic acid at a concentration between 1.80E−06 mM and 1.80E−01 mM;
      xxiv. Myristic acid at a concentration between 2.19E−06 mM and 2.19E−01 mM;
      xxv. Oleic acid at a concentration between 1.77E−06 mM and 1.77E−01 mM;
      xxvi. Palmitic acid at a concentration between 1.95E−06 mM and 1.95E−01 mM;
      xxvii. Stearic acid at a concentration between 1.76E−06 mM and 1.76E−01 mM;
      xxviii. Cholesterol at a concentration between 2.84E−05 mM and 2.84E+00 mM;
      xxix. Tween-80 at a concentration between 8.40E−05 mM and 8.40E+00 mM;
      xxx. Tocopherol acetate at a concentration between 7.40E−06 mM and 7.40E−01 mM;
      xxxi. Pluronic F-68 at a concentration between 5.95E−04 mM and 5.95E+01 mM;
      xxxii. Insulin at a concentration between 1.72E−05 mM and 1.72E+00 mM;
      xxxiii. Transferrin at a concentration between 6.88E−07 mM and 6.88E−02 mM;
      xxxiv. Sodium Selenite at a concentration between 3.87E−07 mM and 3.87E−02 mM;
      xxxv. myo-Inositol at a concentration between 2.00E−03 mM and 2.00E+02 mM;
      xxxvi. Glucose at a concentration between 2.22E−01 mM and 2.22E+04 mM;
      xxxvii. Human Serum Albumin (HSA) at a concentration between 1.50E−05 mM and 1.50E+00 mM;
      xxxviii. Biotin at a concentration between 8.10E−06 mM and 8.10E−01 mM;
      xxxix. Folic Acid at a concentration between 2.40E−04 mM and 2.40E+01 mM;
      xL. Vitamin B12 at a concentration between 1.00E−02 mM and 1.00E+03 mM;
      xLi. Vitamin C at a concentration between 2.84E−03 mM and 2.84E+02 mM;
      xLii. Vitamin E at a concentration between 2.40E−03 mM and 2.40E+02 mM;
      xLiii. Zinc at a concentration between 1.50E−08 mM and 1.50E−03 mM;
      xLiv. L-Ornithine at a concentration between 5.00E−04 mM and 5.00E+01 mM;
      xLv. Creatine Anhydrous at a concentration between 1.00E−02 mM and 1.00E+03 mM;
      xLvi. PolyVinyl Alcohol (PVA) at a concentration between 1.16E−01 and 1.16E+04 mM;
      xLvii. Ethanolamine at a concentration between 5.00E−04 and 5.00E+01 mM;
      xLviii. 2-mercaptoethanol (2-ME) at a concentration between 1.00E−03 mM and 1.00E+02 mM;
      xLix. Androstenedione at a concentration between 1.00E−05 and 1.00E+00 mM;
      L. Dextran at a concentration between 1.00E+01 and 1.00E+06 mg/L;
      Li. L-Alanyl-L-Glutamine (Glutamax) at a concentration between 2.50E−02 mM and 2.50E+03 mM;
      Lii. a polyamine supplement at a concentration between 0.01× and 1000× of a 1000× stock (polyamine supplement being Sigma-Aldrich Catalog #P8483 or equivalent); and
      Liii. an antioxidant supplement at a concentration between 0.01× and 1000× of a 1000× (antioxidant supplement being Sigma-Aldrich Catalog #A1345 or equivalent); and
   b. a second media formulation comprising the first media formulation and at least three of the following components:
      Liv. Nicotinamide at a concentration between 2.50E−02 mM and 2.50E+03 mM;
      Lv. IGF-1 (Insulin like Growth Factor 1) at a concentration between 2.60E−08 mM and 2.60E−03 mM;
      Lvi. 4-1BBL [4-1BB Ligand or ligand for Cluster of Differentiation 137 (CD137)] at a concentration between 4.20E−09 mM and 4.20E−04 mM;
      Lvii. Ashwagandha (botanical name: Withania Somnifera) plant extract at a concentration between 6.00E−01 mg/L and 6.00E+04 mg/L;
      Lviii. Kumquat (botanical name: Fortunella Japonica) plant extract at a concentration between 1.40E−22 mg/L and 1.40E−02 mg/L;
      Lix. Umckaloabo (botanical name: Pelargonium sidoides) plant extract at a concentration between 5.00E−01 mg/L and 5.00E+05 mg/L;
      Lx. Echinacea (botanical name: Echinacea Purpurea) plant extract at a concentration between 1.01E−01 mg/L and 1.00E+05 mg/L;
      Lxi. Red Ginseng (botanical name: Panax ginseng) plant extract at a concentration between 8.43E−02 mg/L and 8.43E+03 mg/L;
      Lxii. 2,3-Butanediol at a concentration between 9.01E−02 mg/L and 9.01E+03 mg/L; and
      Lxiii. Elderberry (botanical name: Sambucus nigra) plant extract at a concentration between 1.06E−01 mg/L and 1.06E+04 mg/L;
   wherein the NK cells are cultured in the first media formulation prior to culture in the second media formulation.
Embodiment 2 provides a one-part media formulation for culturing Natural Killer (NK) cells comprising at least six of the following components:
   i. Glycine at a concentration between 3.50E−03 mM and 3.50E+02 mM;
   ii. L-Alanine at a concentration between 1.10E−02 mM and 1.10E+03 mM;
   iii. L-Asparagine at a concentration between 1.50E−03 mM and 1.50E+02 mM;
   iv. L-Aspartic acid at a concentration between 1.50E−03 mM and 1.50E+02 mM;
   v. L-Glutamic Acid at a concentration between 1.50E−03 mM and 1.50E+02 mM;
   vi. L-Proline at a concentration between 2.50E−03 mM and 2.50E+02 mM;
   vii. L-Serine at a concentration between 3.50E−03 mM and 3.50E+02 mM;
   viii. L-Arginine HCl at a concentration between 1.30E−02 mM and 1.30E+03 mM;
   ix. L-Cystine at a concentration between 2.00E−03 mM and 2.00E+02 mM;
   x. L-Histidine HCl—H₂O at a concentration between 3.50E−03 mM and 3.50E+02 mM;
   xi. L-Isoleucine at a concentration between 8.20E−03 mM and 8.20E+02 mM;
   xii. L-Leucine at a concentration between 8.50E−03 mM and 8.50E+02 mM;
   xiii. L-Lysine hydrochloride at a concentration between 8.96E−03 mM and 8.96E+02 mM;
   xiv. L-Methionine at a concentration between 2.21E−03 mM and 2.21E+02 mM;
   xv. L-Phenylalanine at a concentration between 4.20E−03 mM and 4.20E+02 mM;
   xvi. L-Threonine at a concentration between 8.50E−03 mM and 8.50E+02 mM;
   xvii. L-Tryptophan at a concentration between 9.00E−04 mM and 9.00E+01 mM;
   xviii. L-Tyrosine at a concentration between 4.09E−03 mM and 4.09E+02 mM;
   xix. L-Valine at a concentration between 8.50E−03 mM and 8.50E+02 mM;
   xx. Sodium Pyruvate at a concentration between 1.00E−02 mM and 1.00E+03 mM;
   xxi. Arachidonic acid at a concentration between 3.28E−07 mM and 3.28E−02 mM;
   xxii. Linoleic acid at a concentration between 3.78E−06 mM and 3.78E−01 mM;
   xxiii. Linolenic acid at a concentration between 1.80E−06 mM and 1.80E−01 mM;
   xxiv. Myristic acid at a concentration between 2.19E−06 mM and 2.19E−01 mM;
   xxv. Oleic acid at a concentration between 1.77E−06 mM and 1.77E−01 mM;
   xxvi. Palmitic acid at a concentration between 1.95E−06 mM and 1.95E−01 mM;
   xxvii. Stearic acid at a concentration between 1.76E−06 mM and 1.76E−01 mM;
   xxviii. Cholesterol at a concentration between 2.84E−05 mM and 2.84E+00 mM;
   xxix. Tween-80 at a concentration between 8.40E−05 mM and 8.40E+00 mM;
   xxx. Tocopherol acetate at a concentration between 7.40E−06 mM and 7.40E−01 mM;
   xxxi. Pluronic F-68 at a concentration between 5.95E−04 mM and 5.95E+01 mM;
   xxxii. Insulin at a concentration between 1.72E−05 mM and 1.72E+00 mM;
   xxxiii. Transferrin at a concentration between 6.88E−07 mM and 6.88E−02 mM;
   xxxiv. Sodium Selenite at a concentration between 3.87E−07 mM and 3.87E−02 mM;
   xxxv. myo-Inositol at a concentration between 2.00E−03 mM and 2.00E+02 mM;
   xxxvi. Glucose at a concentration between 2.22E−01 mM and 2.22E+04 mM;
   xxxvii. Human Serum Albumin (HSA) at a concentration between 1.50E−05 mM and 1.50E+00 mM;
   xxxviii. Biotin at a concentration between 8.10E−06 mM and 8.10E−01 mM;
   xxxix. Folic Acid at a concentration between 2.40E−04 mM and 2.40E+01 mM;
   xL. Vitamin B12 at a concentration between 1.00E−02 mM and 1.00E+03 mM;
   xLi. Vitamin C at a concentration between 2.84E−03 mM and 2.84E+02 mM;
   xLii. Vitamin E at a concentration between 2.40E−03 mM and 2.40E+02 mM;
   xLiii. Zinc at a concentration between 1.50E−08 mM and 1.50E−03 mM;
   xLiv. L-Ornithine at a concentration between 5.00E−04 mM and 5.00E+01 mM;
   xLv. Creatine Anhydrous at a concentration between 1.00E−02 mM and 1.00E+03 mM;
   xLvi. PolyVinyl Alcohol (PVA) at a concentration between 1.16E−01 and 1.16E+04 mM;
   xLvii. Ethanolamine at a concentration between 5.00E−04 and 5.00E+01 mM;
   xLviii. 2-mercaptoethanol (2-ME) at a concentration between 1.00E−03 mM and 1.00E+02 mM;
   xLix. Androstenedione at a concentration between 1.00E−05 and 1.00E+00 mM;
   L. Dextran at a concentration between 1.00E+01 and 1.00E+06 mg/L;
   Li. L-Alanyl-L-Glutamine (Glutamax) at a concentration between 2.50E−02 mM and 2.50E+03 mM;
   Lii. a polyamine supplement at a concentration between 0.01× and 1000× of a 1000× stock (polyamine supplement being Sigma-Aldrich Catalog #P8483 or equivalent); and
   Liii. an antioxidant supplement at a concentration between 0.01× and 1000× of a 1000× (antioxidant supplement being Sigma-Aldrich Catalog #A1345 or equivalent).
Embodiment 3 provides a one-part media formulation culturing Natural Killer (NK) cells comprising at least nine of the following components:
   i. Glycine at a concentration between 3.50E−03 mM and 3.50E+02 mM;
   ii. L-Alanine at a concentration between 1.10E−02 mM and 1.10E+03 mM;
   iii. L-Asparagine at a concentration between 1.50E−03 mM and 1.50E+02 mM;
   iv. L-Aspartic acid at a concentration between 1.50E−03 mM and 1.50E+02 mM;
   v. L-Glutamic Acid at a concentration between 1.50E−03 mM and 1.50E+02 mM;
   vi. L-Proline at a concentration between 2.50E−03 mM and 2.50E+02 mM;
   vii. L-Serine at a concentration between 3.50E−03 mM and 3.50E+02 mM;
   viii. L-Arginine HCl at a concentration between 1.30E−02 mM and 1.30E+03 mM;
   ix. L-Cystine at a concentration between 2.00E−03 mM and 2.00E+02 mM;
   x. L-Histidine HCl—H₂O at a concentration between 3.50E−03 mM and 3.50E+02 mM;
   xi. L-Isoleucine at a concentration between 8.20E−03 mM and 8.20E+02 mM;
   xii. L-Leucine at a concentration between 8.50E−03 mM and 8.50E+02 mM;
   xiii. L-Lysine hydrochloride at a concentration between 8.96E−03 mM and 8.96E+02 mM;
   xiv. L-Methionine at a concentration between 2.21E−03 mM and 2.21E+02 mM;
   xv. L-Phenylalanine at a concentration between 4.20E−03 mM and 4.20E+02 mM;
   xvi. L-Threonine at a concentration between 8.50E−03 mM and 8.50E+02 mM;
   xvii. L-Tryptophan at a concentration between 9.00E−04 mM and 9.00E+01 mM;
   xviii. L-Tyrosine at a concentration between 4.09E−03 mM and 4.09E+02 mM;
   xix. L-Valine at a concentration between 8.50E−03 mM and 8.50E+02 mM;
   xx. Sodium Pyruvate at a concentration between 1.00E−02 mM and 1.00E+03 mM;
   xxi. Arachidonic acid at a concentration between 3.28E−07 mM and 3.28E−02 mM;
   xxii. Linoleic acid at a concentration between 3.78E−06 mM and 3.78E−01 mM;
   xxiii. Linolenic acid at a concentration between 1.80E−06 mM and 1.80E−01 mM;
   xxiv. Myristic acid at a concentration between 2.19E−06 mM and 2.19E−01 mM;
   xxv. Oleic acid at a concentration between 1.77E−06 mM and 1.77E−01 mM;
   xxvi. Palmitic acid at a concentration between 1.95E−06 mM and 1.95E−01 mM;
   xxvii. Stearic acid at a concentration between 1.76E−06 mM and 1.76E−01 mM;
   xxviii. Cholesterol at a concentration between 2.84E−05 mM and 2.84E+00 mM;
   xxix. Tween-80 at a concentration between 8.40E−05 mM and 8.40E+00 mM;
   xxx. Tocopherol acetate at a concentration between 7.40E−06 mM and 7.40E−01 mM;
   xxxi. Pluronic F-68 at a concentration between 5.95E−04 mM and 5.95E+01 mM;
   xxxii. Insulin at a concentration between 1.72E−05 mM and 1.72E+00 mM;
   xxxiii. Transferrin at a concentration between 6.88E−07 mM and 6.88E−02 mM;
   xxxiv. Sodium Selenite at a concentration between 3.87E−07 mM and 3.87E−02 mM;
   xxxv. myo-Inositol at a concentration between 2.00E−03 mM and 2.00E+02 mM;
   xxxvi. Glucose at a concentration between 2.22E−01 mM and 2.22E+04 mM;
   xxxvii. Human Serum Albumin (HSA) at a concentration between 1.50E−05 mM and 1.50E+00 mM;
   xxxviii. Biotin at a concentration between 8.10E−06 mM and 8.10E−01 mM;
   xxxix. Folic Acid at a concentration between 2.40E−04 mM and 2.40E+01 mM;
   xL. Vitamin B12 at a concentration between 1.00E−02 mM and 1.00E+03 mM;
   xLi. Vitamin C at a concentration between 2.84E−03 mM and 2.84E+02 mM;
   xLii. Vitamin E at a concentration between 2.40E−03 mM and 2.40E+02 mM;
   xLiii. Zinc at a concentration between 1.50E−08 mM and 1.50E−03 mM;
   xLiv. L-Ornithine at a concentration between 5.00E−04 mM and 5.00E+01 mM;
   xLv. Creatine Anhydrous at a concentration between 1.00E−02 mM and 1.00E+03 mM;
   xLvi. PolyVinyl Alcohol (PVA) at a concentration between 1.16E−01 and 1.16E+04 mM;
   xLvii. Ethanolamine at a concentration between 5.00E−04 and 5.00E+01 mM;
   xLviii. 2-mercaptoethanol (2-ME) at a concentration between 1.00E−03 mM and 1.00E+02 mM;
   xLix. Androstenedione at a concentration between 1.00E−05 and 1.00E+00 mM;
   L. Dextran at a concentration between 1.00E+01 and 1.00E+06 mg/L;
   Li. L-Alanyl-L-Glutamine (Glutamax) at a concentration between 2.50E−02 mM and 2.50E+03 mM;
   Lii. a polyamine supplement at a concentration between 0.01× and 1000× of a 1000× stock (polyamine supplement being Sigma-Aldrich Catalog #P8483 or equivalent);
   Liii. an antioxidant supplement at a concentration between 0.01× and 1000× of a 1000× (antioxidant supplement being Sigma-Aldrich Catalog #A1345 or equivalent);
   Liv. Nicotinamide at a concentration between 2.50E−02 mM and 2.50E+03 mM;
   Lv. IGF-1 (Insulin like Growth Factor 1) at a concentration between 2.60E−08 mM and 2.60E−03 mM;
   Lvi. 4-1BBL [4-1BB Ligand or ligand for Cluster of Differentiation 137 (CD137)] at a concentration between 4.20E−09 mM and 4.20E−04 mM;
   Lvii. Ashwagandha (botanical name: Withania Somnifera) plant extract at a concentration between 6.00E−01 mg/L and 6.00E+04 mg/L;
   Lviii. Kumquat (botanical name: Fortunella Japonica) plant extract at a concentration between 1.40E−22 mg/L and 1.40E−02 mg/L;
   Lix. Umckaloabo (botanical name: Pelargonium sidoides) plant extract at a concentration between 5.00E−01 mg/L and 5.00E+05 mg/L;
   Lx. Echinacea (botanical name: Echinacea Purpurea) plant extract at a concentration between 1.01E−01 mg/L and 1.00E+05 mg/L;
   Lxi. Red Ginseng (botanical name: Panax ginseng) plant extract at a concentration between 8.43E−02 mg/L and 8.43E+03 mg/L;
   Lxii. 2,3-Butanediol at a concentration between 9.01E−02 mg/L and 9.01E+03 mg/L; and
   Lxiii. Elderberry (botanical name: Sambucus nigra) plant extract at a concentration between 1.06E−01 mg/L and 1.06E+04 mg/L
Embodiment 4 provides the formulation of any one of embodiments 1-3, wherein the formulation is added to a base cell culture media as a supplement.
Embodiment 5 provides a method for culturing Natural Killer (NK) cells or precursor cells thereof comprising culturing the NK cells or precursor cells thereof in a cell culture media formulation comprising:
a. a first media formulation comprising at least six of the following components:
   i. Glycine at a concentration between 3.50E−03 mM and 3.50E+02 mM;
   ii. L-Alanine at a concentration between 1.10E−02 mM and 1.10E+03 mM;
   iii. L-Asparagine at a concentration between 1.50E−03 mM and 1.50E+02 mM;
   iv. L-Aspartic acid at a concentration between 1.50E−03 mM and 1.50E+02 mM;
   v. L-Glutamic Acid at a concentration between 1.50E−03 mM and 1.50E+02 mM;
   vi. L-Proline at a concentration between 2.50E−03 mM and 2.50E+02 mM;
   vii. L-Serine at a concentration between 3.50E−03 mM and 3.50E+02 mM;
   viii. L-Arginine HCl at a concentration between 1.30E−02 mM and 1.30E+03 mM;
   ix. L-Cystine at a concentration between 2.00E−03 mM and 2.00E+02 mM;
   x. L-Histidine HCl—H₂O at a concentration between 3.50E−03 mM and 3.50E+02 mM;
   xi. L-Isoleucine at a concentration between 8.20E−03 mM and 8.20E+02 mM;
   xii. L-Leucine at a concentration between 8.50E−03 mM and 8.50E+02 mM;
   xiii. L-Lysine hydrochloride at a concentration between 8.96E−03 mM and 8.96E+02 mM;
   xiv. L-Methionine at a concentration between 2.21E−03 mM and 2.21E+02 mM;
   xv. L-Phenylalanine at a concentration between 4.20E−03 mM and 4.20E+02 mM;
   xvi. L-Threonine at a concentration between 8.50E−03 mM and 8.50E+02 mM;
   xvii. L-Tryptophan at a concentration between 9.00E−04 mM and 9.00E+01 mM;
   xviii. L-Tyrosine at a concentration between 4.09E−03 mM and 4.09E+02 mM;
   xix. L-Valine at a concentration between 8.50E−03 mM and 8.50E+02 mM;
   xx. Sodium Pyruvate at a concentration between 1.00E−02 mM and 1.00E+03 mM;
   xxi. Arachidonic acid at a concentration between 3.28E−07 mM and 3.28E−02 mM;
   xxii. Linoleic acid at a concentration between 3.78E−06 mM and 3.78E−01 mM;
   xxiii. Linolenic acid at a concentration between 1.80E−06 mM and 1.80E−01 mM;
   xxiv. Myristic acid at a concentration between 2.19E−06 mM and 2.19E−01 mM;
   xxv. Oleic acid at a concentration between 1.77E−06 mM and 1.77E−01 mM;
   xxvi. Palmitic acid at a concentration between 1.95E−06 mM and 1.95E−01 mM;
   xxvii. Stearic acid at a concentration between 1.76E−06 mM and 1.76E−01 mM;
   xxviii. Cholesterol at a concentration between 2.84E−05 mM and 2.84E+00 mM;
   xxix. Tween-80 at a concentration between 8.40E−05 mM and 8.40E+00 mM;
   xxx. Tocopherol acetate at a concentration between 7.40E−06 mM and 7.40E−01 mM;
   xxxi. Pluronic F-68 at a concentration between 5.95E−04 mM and 5.95E+01 mM;
   xxxii. Insulin at a concentration between 1.72E−05 mM and 1.72E+00 mM;
   xxxiii. Transferrin at a concentration between 6.88E−07 mM and 6.88E−02 mM;
   xxxiv. Sodium Selenite at a concentration between 3.87E−07 mM and 3.87E−02 mM;
   xxxv. myo-Inositol at a concentration between 2.00E−03 mM and 2.00E+02 mM;
   xxxvi. Glucose at a concentration between 2.22E−01 mM and 2.22E+04 mM;
   xxxvii. Human Serum Albumin (HSA) at a concentration between 1.50E−05 mM and 1.50E+00 mM;
   xxxviii. Biotin at a concentration between 8.10E−06 mM and 8.10E−01 mM;
   xxxix. Folic Acid at a concentration between 2.40E−04 mM and 2.40E+01 mM;
   xL. Vitamin B12 at a concentration between 1.00E−02 mM and 1.00E+03 mM;
   xLi. Vitamin C at a concentration between 2.84E−03 mM and 2.84E+02 mM;
   xLii. Vitamin E at a concentration between 2.40E−03 mM and 2.40E+02 mM;
   xLiii. Zinc at a concentration between 1.50E−08 mM and 1.50E−03 mM;
   xLiv. L-Ornithine at a concentration between 5.00E−04 mM and 5.00E+01 mM;
   xLv. Creatine Anhydrous at a concentration between 1.00E−02 mM and 1.00E+03 mM;
   xLvi. PolyVinyl Alcohol (PVA) at a concentration between 1.16E−01 and 1.16E+04 mM;
   xLvii. Ethanolamine at a concentration between 5.00E−04 and 5.00E+01 mM;
   xLviii. 2-mercaptoethanol (2-ME) at a concentration between 1.00E−03 mM and 1.00E+02 mM;
   xLix. Androstenedione at a concentration between 1.00E−05 and 1.00E+00 mM;
   L. Dextran at a concentration between 1.00E+01 and 1.00E+06 mg/L;
   Li. L-Alanyl-L-Glutamine (Glutamax) at a concentration between 2.50E−02 mM and 2.50E+03 mM;
   Lii. a polyamine supplement at a concentration between 0.01× and 1000× of a 1000× stock (polyamine supplement being Sigma-Aldrich Catalog #P8483 or equivalent); and
   Liii. an antioxidant supplement at a concentration between 0.01× and 1000× of a 1000× (antioxidant supplement being Sigma-Aldrich Catalog #A1345 or equivalent); and
b. a second media formulation comprising the first media formulation and at least three of the following components:
   Liv. Nicotinamide at a concentration between 2.50E−02 mM and 2.50E+03 mM;
   Lv. IGF-1 (Insulin like Growth Factor 1) at a concentration between 2.60E−08 mM and 2.60E−03 mM;
   Lvi. 4-1BBL [4-1BB Ligand or ligand for Cluster of Differentiation 137 (CD137)] at a concentration between 4.20E−09 mM and 4.20E−04 mM;
   Lvii. Ashwagandha (botanical name: Withania Somnifera) plant extract at a concentration between 6.00E−01 mg/L and 6.00E+04 mg/L;
   Lviii. Kumquat (botanical name: Fortunella Japonica) plant extract at a concentration between 1.40E−22 mg/L and 1.40E−02 mg/L;
   Lix. Umckaloabo (botanical name: Pelargonium sidoides) plant extract at a concentration between 5.00E−01 mg/L and 5.00E+05 mg/L;
   Lx. Echinacea (botanical name: Echinacea Purpurea) plant extract at a concentration between 1.01E−01 mg/L and 1.00E+05 mg/L;
   Lxi. Red Ginseng (botanical name: Panax ginseng) plant extract at a concentration between 8.43E−02 mg/L and 8.43E+03 mg/L;
   Lxii. 2,3-Butanediol at a concentration between 9.01E−02 mg/L and 9.01E+03 mg/L; and
   Lxiii. Elderberry (botanical name: Sambucus nigra) plant extract at a concentration between 1.06E−01 mg/L and 1.06E+04 mg/L.
Embodiment 6 provides the method of embodiment 5, wherein the NK cells or precursor cells thereof are cultured in the first media formulation.
Embodiment 7 provides the method of embodiment 5, wherein the NK cells or precursor cells thereof are cultured in the second media formulation.
Embodiment 8 provides the method of embodiment 5, wherein the NK cells or precursor cells thereof are cultured in the first media formulation followed by the second formulation.
Embodiment 9 provides the method of embodiment 5, wherein the NK cells or precursor cells thereof are cultured ex vivo.
Embodiment 10 provides the method of embodiment 5, wherein the NK cells or precursor cells thereof are cultured in vitro.
Embodiment 11 provides the method of embodiment 5, wherein cytotoxic activity of the NK cells against target cells is enhanced.
Embodiment 12 provides the method of embodiment 11, wherein the target cells are cancer cells.
Embodiment 13 provides the method of embodiment 5, wherein activation of the NK cells is enhanced.
Embodiment 14 provides the method of embodiment 5, wherein dysfunction of the NK cells is reduced.
Embodiment 15 provides the method of embodiment 14, wherein the dysfunction is exhaustion.
Embodiment 16 provides the method of embodiment 5, wherein metabolic function of the NK cells is enhanced.
Embodiment 17 provides the method of embodiment 5, wherein high viability and cell-proliferation of NK cells is achieved.
Embodiment 18 provides the method of embodiment 5, wherein use of serum is eliminated or reduced.
Embodiment 19 provides the method of embodiment 5, wherein use of cytokine supplements is eliminated or reduced.
Embodiment 20 provides the method of embodiment 5, wherein the use of Antigen Presenting Cells (APCs) or feeder cells is eliminated or reduced.
Embodiment 21 provides the method of embodiment 5, wherein the culture of NK precursor cells induces their differentiation.
Embodiment 22 provides the method of embodiment 5, wherein culture of NK cells or precursor cells modulates the distribution and relative populations of NK cell subsets.
Embodiment 23 provides the method of embodiment 22, wherein the NK cell subsets are memory NK cells.
Embodiment 24 provides the method of embodiment 22, wherein the NK cell subsets are defined by relative expression of activating and inhibitory receptors.
Embodiment 25 provides a method for cryopreservation of Natural Killer (NK) cells, wherein freezing the NK cells is accomplished in a cryopreservation medium comprising:
   a. a first media formulation comprising at least six of the following components:
      i. Glycine at a concentration between 3.50E−03 mM and 3.50E+02 mM;
      ii. L-Alanine at a concentration between 1.10E−02 mM and 1.10E+03 mM;
      iii. L-Asparagine at a concentration between 1.50E−03 mM and 1.50E+02 mM;
      iv. L-Aspartic acid at a concentration between 1.50E−03 mM and 1.50E+02 mM;
      v. L-Glutamic Acid at a concentration between 1.50E−03 mM and 1.50E+02 mM;
      vi. L-Proline at a concentration between 2.50E−03 mM and 2.50E+02 mM;
      vii. L-Serine at a concentration between 3.50E−03 mM and 3.50E+02 mM;
      viii. L-Arginine HCl at a concentration between 1.30E−02 mM and 1.30E+03 mM;
      ix. L-Cystine at a concentration between 2.00E−03 mM and 2.00E+02 mM;
      x. L-Histidine HCl—H₂O at a concentration between 3.50E−03 mM and 3.50E+02 mM;
      xi. L-Isoleucine at a concentration between 8.20E−03 mM and 8.20E+02 mM;
      xii. L-Leucine at a concentration between 8.50E−03 mM and 8.50E+02 mM;
      xiii. L-Lysine hydrochloride at a concentration between 8.96E−03 mM and 8.96E+02 mM;
      xiv. L-Methionine at a concentration between 2.21E−03 mM and 2.21E+02 mM;
      xv. L-Phenylalanine at a concentration between 4.20E−03 mM and 4.20E+02 mM;
      xvi. L-Threonine at a concentration between 8.50E−03 mM and 8.50E+02 mM;
      xvii. L-Tryptophan at a concentration between 9.00E−04 mM and 9.00E+01 mM;
      xviii. L-Tyrosine at a concentration between 4.09E−03 mM and 4.09E+02 mM;
      xix. L-Valine at a concentration between 8.50E−03 mM and 8.50E+02 mM;
      xx. Sodium Pyruvate at a concentration between 1.00E−02 mM and 1.00E+03 mM;
      xxi. Arachidonic acid at a concentration between 3.28E−07 mM and 3.28E−02 mM;
      xxii. Linoleic acid at a concentration between 3.78E−06 mM and 3.78E−01 mM;
      xxiii. Linolenic acid at a concentration between 1.80E−06 mM and 1.80E−01 mM;
      xxiv. Myristic acid at a concentration between 2.19E−06 mM and 2.19E−01 mM;
      xxv. Oleic acid at a concentration between 1.77E−06 mM and 1.77E−01 mM;
      xxvi. Palmitic acid at a concentration between 1.95E−06 mM and 1.95E−01 mM;
      xxvii. Stearic acid at a concentration between 1.76E−06 mM and 1.76E−01 mM;
      xxviii. Cholesterol at a concentration between 2.84E−05 mM and 2.84E+00 mM;
      xxix. Tween-80 at a concentration between 8.40E−05 mM and 8.40E+00 mM;
      xxx. Tocopherol acetate at a concentration between 7.40E−06 mM and 7.40E−01 mM;
      xxxi. Pluronic F-68 at a concentration between 5.95E−04 mM and 5.95E+01 mM;
      xxxii. Insulin at a concentration between 1.72E−05 mM and 1.72E+00 mM;
      xxxiii. Transferrin at a concentration between 6.88E−07 mM and 6.88E−02 mM;
      xxxiv. Sodium Selenite at a concentration between 3.87E−07 mM and 3.87E−02 mM;
      xxxv. myo-Inositol at a concentration between 2.00E−03 mM and 2.00E+02 mM;
      xxxvi. Glucose at a concentration between 2.22E−01 mM and 2.22E+04 mM;
      xxxvii. Human Serum Albumin (HSA) at a concentration between 1.50E−05 mM and 1.50E+00 mM;
      xxxviii. Biotin at a concentration between 8.10E−06 mM and 8.10E−01 mM;
      xxxix. Folic Acid at a concentration between 2.40E−04 mM and 2.40E+01 mM;
      xL. Vitamin B12 at a concentration between 1.00E−02 mM and 1.00E+03 mM;
      xLi. Vitamin C at a concentration between 2.84E−03 mM and 2.84E+02 mM;
      xLii. Vitamin E at a concentration between 2.40E−03 mM and 2.40E+02 mM;
      xLiii. Zinc at a concentration between 1.50E−08 mM and 1.50E−03 mM;
      xLiv. L-Ornithine at a concentration between 5.00E−04 mM and 5.00E+01 mM;
      xLv. Creatine Anhydrous at a concentration between 1.00E−02 mM and 1.00E+03 mM;
      xLvi. PolyVinyl Alcohol (PVA) at a concentration between 1.16E−01 and 1.16E+04 mM;
      xLvii. Ethanolamine at a concentration between 5.00E−04 and 5.00E+01 mM;
      xLviii. 2-mercaptoethanol (2-ME) at a concentration between 1.00E−03 mM and 1.00E+02 mM;
      xLix. Androstenedione at a concentration between 1.00E−05 and 1.00E+00 mM;
      L. Dextran at a concentration between 1.00E+01 and 1.00E+06 mg/L;
      Li. L-Alanyl-L-Glutamine (Glutamax) at a concentration between 2.50E−02 mM and 2.50E+03 mM;
      Lii. a polyamine supplement at a concentration between 0.01× and 1000× of a 1000× stock (polyamine supplement being Sigma-Aldrich Catalog #P8483 or equivalent); and
      Liii. an antioxidant supplement at a concentration between 0.01× and 1000× of a 1000× (antioxidant supplement being Sigma-Aldrich Catalog #A1345 or equivalent); and
   b. a second media formulation comprising the first media formulation and at least three of the following components:
      Liv. Nicotinamide at a concentration between 2.50E−02 mM and 2.50E+03 mM;
      Lv. IGF-1 (Insulin like Growth Factor 1) at a concentration between 2.60E−08 mM and 2.60E−03 mM;
      Lvi. 4-1BBL [4-1BB Ligand or ligand for Cluster of Differentiation 137 (CD137)] at a concentration between 4.20E−09 mM and 4.20E−04 mM;
      Lvii. Ashwagandha (botanical name: Withania Somnifera) plant extract at a concentration between 6.00E−01 mg/L and 6.00E+04 mg/L;
      Lviii. Kumquat (botanical name: Fortunella Japonica) plant extract at a concentration between 1.40E−22 mg/L and 1.40E−02 mg/L;
      Lix. Umckaloabo (botanical name: Pelargonium sidoides) plant extract at a concentration between 5.00E−01 mg/L and 5.00E+05 mg/L;
      Lx. Echinacea (botanical name: Echinacea Purpurea) plant extract at a concentration between 1.01E−01 mg/L and 1.00E+05 mg/L;
      Lxi. Red Ginseng (botanical name: Panax ginseng) plant extract at a concentration between 8.43E−02 mg/L and 8.43E+03 mg/L;
      Lxii. 2,3-Butanediol at a concentration between 9.01E−02 mg/L and 9.01E+03 mg/L; and
      Lxiii. Elderberry (botanical name: Sambucus nigra) plant extract at a concentration between 1.06E−01 mg/L and 1.06E+04 mg/L.
Embodiment 26 provides the method of embodiment 25, wherein the cryopreservation medium comprises the first media formulation.
Embodiment 27 provides the method of embodiment 25, wherein the cryopreservation medium comprises the second media formulation.
Embodiment 28 provides a method for transduction of Natural Killer (NK) immune cells comprising
a first cell culture step comprising culturing the NK cells in a cell culture medium comprising:
a. a first media formulation comprising at least six of the following components:
   i. Glycine at a concentration between 3.50E−03 mM and 3.50E+02 mM;
   ii. L-Alanine at a concentration between 1.10E−02 mM and 1.10E+03 mM;
   iii. L-Asparagine at a concentration between 1.50E−03 mM and 1.50E+02 mM;
   iv. L-Aspartic acid at a concentration between 1.50E−03 mM and 1.50E+02 mM;
   v. L-Glutamic Acid at a concentration between 1.50E−03 mM and 1.50E+02 mM;
   vi. L-Proline at a concentration between 2.50E−03 mM and 2.50E+02 mM;
   vii. L-Serine at a concentration between 3.50E−03 mM and 3.50E+02 mM;
   viii. L-Arginine HCl at a concentration between 1.30E−02 mM and 1.30E+03 mM;
   ix. L-Cystine at a concentration between 2.00E−03 mM and 2.00E+02 mM;
   x. L-Histidine HCl—H₂O at a concentration between 3.50E−03 mM and 3.50E+02 mM;
   xi. L-Isoleucine at a concentration between 8.20E−03 mM and 8.20E+02 mM;
   xii. L-Leucine at a concentration between 8.50E−03 mM and 8.50E+02 mM;
   xiii. L-Lysine hydrochloride at a concentration between 8.96E−03 mM and 8.96E+02 mM;
   xiv. L-Methionine at a concentration between 2.21E−03 mM and 2.21E+02 mM;
   xv. L-Phenylalanine at a concentration between 4.20E−03 mM and 4.20E+02 mM;
   xvi. L-Threonine at a concentration between 8.50E−03 mM and 8.50E+02 mM;
   xvii. L-Tryptophan at a concentration between 9.00E−04 mM and 9.00E+01 mM;
   xviii. L-Tyrosine at a concentration between 4.09E−03 mM and 4.09E+02 mM;
   xix. L-Valine at a concentration between 8.50E−03 mM and 8.50E+02 mM;
   xx. Sodium Pyruvate at a concentration between 1.00E−02 mM and 1.00E+03 mM;
   xxi. Arachidonic acid at a concentration between 3.28E−07 mM and 3.28E−02 mM;
   xxii. Linoleic acid at a concentration between 3.78E−06 mM and 3.78E−01 mM;
   xxiii. Linolenic acid at a concentration between 1.80E−06 mM and 1.80E−01 mM;
   xxiv. Myristic acid at a concentration between 2.19E−06 mM and 2.19E−01 mM;
   xxv. Oleic acid at a concentration between 1.77E−06 mM and 1.77E−01 mM;
   xxvi. Palmitic acid at a concentration between 1.95E−06 mM and 1.95E−01 mM;
   xxvii. Stearic acid at a concentration between 1.76E−06 mM and 1.76E−01 mM;
   xxviii. Cholesterol at a concentration between 2.84E−05 mM and 2.84E+00 mM;
   xxix. Tween-80 at a concentration between 8.40E−05 mM and 8.40E+00 mM;
   xxx. Tocopherol acetate at a concentration between 7.40E−06 mM and 7.40E−01 mM;
   xxxi. Pluronic F-68 at a concentration between 5.95E−04 mM and 5.95E+01 mM;
   xxxii. Insulin at a concentration between 1.72E−05 mM and 1.72E+00 mM;
   xxxiii. Transferrin at a concentration between 6.88E−07 mM and 6.88E−02 mM;
   xxxiv. Sodium Selenite at a concentration between 3.87E−07 mM and 3.87E−02 mM;
   xxxv. myo-Inositol at a concentration between 2.00E−03 mM and 2.00E+02 mM;
   xxxvi. Glucose at a concentration between 2.22E−01 mM and 2.22E+04 mM;
   xxxvii. Human Serum Albumin (HSA) at a concentration between 1.50E−05 mM and 1.50E+00 mM;
   xxxviii. Biotin at a concentration between 8.10E−06 mM and 8.10E−01 mM;
   xxxix. Folic Acid at a concentration between 2.40E−04 mM and 2.40E+01 mM;
   xL. Vitamin B12 at a concentration between 1.00E−02 mM and 1.00E+03 mM;
   xLi. Vitamin C at a concentration between 2.84E−03 mM and 2.84E+02 mM;
   xLii. Vitamin E at a concentration between 2.40E−03 mM and 2.40E+02 mM;
   xLiii. Zinc at a concentration between 1.50E−08 mM and 1.50E−03 mM;
   xLiv. L-Ornithine at a concentration between 5.00E−04 mM and 5.00E+01 mM;
   xLv. Creatine Anhydrous at a concentration between 1.00E−02 mM and 1.00E+03 mM;
   xLvi. PolyVinyl Alcohol (PVA) at a concentration between 1.16E−01 and 1.16E+04 mM;
   xLvii. Ethanolamine at a concentration between 5.00E−04 and 5.00E+01 mM;
   xLviii. 2-mercaptoethanol (2-ME) at a concentration between 1.00E−03 mM and 1.00E+02 mM;
   xLix. Androstenedione at a concentration between 1.00E−05 and 1.00E+00 mM;
   L. Dextran at a concentration between 1.00E+01 and 1.00E+06 mg/L;
   Li. L-Alanyl-L-Glutamine (Glutamax) at a concentration between 2.50E−02 mM and 2.50E+03 mM;
   Lii. a polyamine supplement at a concentration between 0.01× and 1000× of a 1000× stock (polyamine supplement being Sigma-Aldrich Catalog #P8483 or equivalent); and
   Liii. an antioxidant supplement at a concentration between 0.01× and 1000× of a 1000× (antioxidant supplement being Sigma-Aldrich Catalog #A1345 or equivalent); and a second media formulation comprising the first media formulation and at least three of the following components:
   Liv. Nicotinamide at a concentration between 2.50E−02 mM and 2.50E+03 mM;
   Lv. IGF-1 (Insulin like Growth Factor 1) at a concentration between 2.60E−08 mM and 2.60E−03 mM;
   Lvi. 4-1BBL [4-1BB Ligand or ligand for Cluster of Differentiation 137 (CD137)] at a concentration between 4.20E−09 mM and 4.20E−04 mM;
   Lvii. Ashwagandha (botanical name: Withania Somnifera) plant extract at a concentration between 6.00E−01 mg/L and 6.00E+04 mg/L;
   Lviii. Kumquat (botanical name: Fortunella Japonica) plant extract at a concentration between 1.40E−22 mg/L and 1.40E−02 mg/L;
   Lix. Umckaloabo (botanical name: Pelargonium sidoides) plant extract at a concentration between 5.00E−01 mg/L and 5.00E+05 mg/L;
   Lx. Echinacea (botanical name: Echinacea Purpurea) plant extract at a concentration between 1.01E−01 mg/L and 1.00E+05 mg/L;
   Lxi. Red Ginseng (botanical name: Panax ginseng) plant extract at a concentration between 8.43E−02 mg/L and 8.43E+03 mg/L;
   Lxii. 2,3-Butanediol at a concentration between 9.01E−02 mg/L and 9.01E+03 mg/L; and
   Lxiii. Elderberry (botanical name: Sambucus nigra) plant extract at a concentration between 1.06E−01 mg/L and 1.06E+04 mg/L;
b. contacting the NK cells with a viral vector; and
c. a second cell culture step comprising culturing the transduced NK cells in a cell culture medium comprising the cell culture medium formulation of step a.
Embodiment 29 provides the method of embodiment 28, wherein the first and second cell cultures steps are carried out in the first cell culture formulation.
Embodiment 30 provides the method of embodiment 28, wherein the first and second cell culture steps are carried out in the second cell culture formulation.
Embodiment 31 provides the method of embodiment 28, wherein the first cell culture step is carried out in the first cell culture formulation and the second cell culture step is carried out in the second cell culture formulation.
Embodiment 32 provides the method of embodiment 28, wherein the vector encodes a chimeric antigen receptor (CAR).
Embodiment 33 provides a method for differentiating Natural Killer (NK) cells from progenitor cells comprising culturing the progenitor cells in a cell culture media comprising:
   a. a first media formulation comprising at least six of the following components:
      i. Glycine at a concentration between 3.50E−03 mM and 3.50E+02 mM;
      ii. L-Alanine at a concentration between 1.10E−02 mM and 1.10E+03 mM;
      iii. L-Asparagine at a concentration between 1.50E−03 mM and 1.50E+02 mM;
      iv. L-Aspartic acid at a concentration between 1.50E−03 mM and 1.50E+02 mM;
      v. L-Glutamic Acid at a concentration between 1.50E−03 mM and 1.50E+02 mM;
      vi. L-Proline at a concentration between 2.50E−03 mM and 2.50E+02 mM;
      vii. L-Serine at a concentration between 3.50E−03 mM and 3.50E+02 mM;
      viii. L-Arginine HCl at a concentration between 1.30E−02 mM and 1.30E+03 mM;
      ix. L-Cystine at a concentration between 2.00E−03 mM and 2.00E+02 mM;
      x. L-Histidine HCl—H₂O at a concentration between 3.50E−03 mM and 3.50E+02 mM;
      xi. L-Isoleucine at a concentration between 8.20E−03 mM and 8.20E+02 mM;
      xii. L-Leucine at a concentration between 8.50E−03 mM and 8.50E+02 mM;
      xiii. L-Lysine hydrochloride at a concentration between 8.96E−03 mM and 8.96E+02 mM;
      xiv. L-Methionine at a concentration between 2.21E−03 mM and 2.21E+02 mM;
      xv. L-Phenylalanine at a concentration between 4.20E−03 mM and 4.20E+02 mM;
      xvi. L-Threonine at a concentration between 8.50E−03 mM and 8.50E+02 mM;
      xvii. L-Tryptophan at a concentration between 9.00E−04 mM and 9.00E+01 mM;
      xviii. L-Tyrosine at a concentration between 4.09E−03 mM and 4.09E+02 mM;
      xix. L-Valine at a concentration between 8.50E−03 mM and 8.50E+02 mM;
      xx. Sodium Pyruvate at a concentration between 1.00E−02 mM and 1.00E+03 mM;
      xxi. Arachidonic acid at a concentration between 3.28E−07 mM and 3.28E−02 mM;
      xxii. Linoleic acid at a concentration between 3.78E−06 mM and 3.78E−01 mM;
      xxiii. Linolenic acid at a concentration between 1.80E−06 mM and 1.80E−01 mM;
      xxiv. Myristic acid at a concentration between 2.19E−06 mM and 2.19E−01 mM;
      xxv. Oleic acid at a concentration between 1.77E−06 mM and 1.77E−01 mM;
      xxvi. Palmitic acid at a concentration between 1.95E−06 mM and 1.95E−01 mM;
      xxvii. Stearic acid at a concentration between 1.76E−06 mM and 1.76E−01 mM;
      xxviii. Cholesterol at a concentration between 2.84E−05 mM and 2.84E+00 mM;
      xxix. Tween-80 at a concentration between 8.40E−05 mM and 8.40E+00 mM;
      xxx. Tocopherol acetate at a concentration between 7.40E−06 mM and 7.40E−01 mM;
      xxxi. Pluronic F-68 at a concentration between 5.95E−04 mM and 5.95E+01 mM;
      xxxii. Insulin at a concentration between 1.72E−05 mM and 1.72E+00 mM;
      xxxiii. Transferrin at a concentration between 6.88E−07 mM and 6.88E−02 mM;
      xxxiv. Sodium Selenite at a concentration between 3.87E−07 mM and 3.87E−02 mM;
      xxxv. myo-Inositol at a concentration between 2.00E−03 mM and 2.00E+02 mM;
      xxxvi. Glucose at a concentration between 2.22E−01 mM and 2.22E+04 mM;
      xxxvii. Human Serum Albumin (HSA) at a concentration between 1.50E−05 mM and 1.50E+00 mM;
      xxxviii. Biotin at a concentration between 8.10E−06 mM and 8.10E−01 mM;
      xxxix. Folic Acid at a concentration between 2.40E−04 mM and 2.40E+01 mM;
      xL. Vitamin B12 at a concentration between 1.00E−02 mM and 1.00E+03 mM;
      xLi. Vitamin C at a concentration between 2.84E−03 mM and 2.84E+02 mM;
      xLii. Vitamin E at a concentration between 2.40E−03 mM and 2.40E+02 mM;
      xLiii. Zinc at a concentration between 1.50E−08 mM and 1.50E−03 mM;
      xLiv. L-Ornithine at a concentration between 5.00E−04 mM and 5.00E+01 mM;
      xLv. Creatine Anhydrous at a concentration between 1.00E−02 mM and 1.00E+03 mM;
      xLvi. PolyVinyl Alcohol (PVA) at a concentration between 1.16E−01 and 1.16E+04 mM;
      xLvii. Ethanolamine at a concentration between 5.00E−04 and 5.00E+01 mM;
      xLviii. 2-mercaptoethanol (2-ME) at a concentration between 1.00E−03 mM and 1.00E+02 mM;
      xLix. Androstenedione at a concentration between 1.00E−05 and 1.00E+00 mM;
      L. Dextran at a concentration between 1.00E+01 and 1.00E+06 mg/L;
      Li. L-Alanyl-L-Glutamine (Glutamax) at a concentration between 2.50E−02 mM and 2.50E+03 mM;
      Lii. a polyamine supplement at a concentration between 0.01× and 1000× of a 1000× stock (polyamine supplement being Sigma-Aldrich Catalog #P8483 or equivalent); and
      Liii. an antioxidant supplement at a concentration between 0.01× and 1000× of a 1000× (antioxidant supplement being Sigma-Aldrich Catalog #A1345 or equivalent); and
   b. a second media formulation comprising the first media formulation and at least three of the following components:
      Liv. Nicotinamide at a concentration between 2.50E−02 mM and 2.50E+03 mM;
      Lv. IGF-1 (Insulin like Growth Factor 1) at a concentration between 2.60E−08 mM and 2.60E−03 mM;
      Lvi. 4-1BBL [4-1BB Ligand or ligand for Cluster of Differentiation 137 (CD137)] at a concentration between 4.20E−09 mM and 4.20E−04 mM;
      Lvii. Ashwagandha (botanical name: Withania Somnifera) plant extract at a concentration between 6.00E−01 mg/L and 6.00E+04 mg/L;
      Lviii. Kumquat (botanical name: Fortunella Japonica) plant extract at a concentration between 1.40E−22 mg/L and 1.40E−02 mg/L;
      Lix. Umckaloabo (botanical name: Pelargonium sidoides) plant extract at a concentration between 5.00E−01 mg/L and 5.00E+05 mg/L;
      Lx. Echinacea (botanical name: Echinacea Purpurea) plant extract at a concentration between 1.01E−01 mg/L and 1.00E+05 mg/L;
      Lxi. Red Ginseng (botanical name: Panax ginseng) plant extract at a concentration between 8.43E−02 mg/L and 8.43E+03 mg/L;
      Lxii. 2,3-Butanediol at a concentration between 9.01E−02 mg/L and 9.01E+03 mg/L; and
      Lxiii. Elderberry (botanical name: Sambucus nigra) plant extract at a concentration between 1.06E−01 mg/L and 1.06E+04 mg/L.
Embodiment 34 provides the method of embodiment 33, wherein the culture media comprises the first media formulation.
Embodiment 35 provides the method of embodiment 33, wherein the culture media comprises the second media formulation.
Embodiment 36 provides the method of embodiment 35, wherein the culture media comprises the first media formulation followed by the second media formulation.
Embodiment 37 provides the method of embodiment 33, wherein the culture of Natural Killer (NK) cells modulates the distribution and relative populations of NK cell subsets.
Embodiment 38 provides the method of embodiment 33, wherein the NK subsets are memory NK cells.
Embodiment 39 provides the method of embodiment 33, wherein the NK cell subsets are defined by relative expression of activating and inhibitory receptors.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A two-part media formulation for culturing Natural Killer (NK) cells comprising:
a. a first media formulation comprising at least six of following components:
i. Glycine at a concentration between 3.50E−03 mM and 3.50E+02 mM;
ii. L-Alanine at a concentration between 1.10E−02 mM and 1.10E+03 mM;
iii. L-Asparagine at a concentration between 1.50E−03 mM and 1.50E+02 mM;
iv. L-Aspartic acid at a concentration between 1.50E−03 mM and 1.50E+02 mM;
v. L-Glutamic Acid at a concentration between 1.50E−03 mM and 1.50E+02 mM;
vi. L-Proline at a concentration between 2.50E−03 mM and 2.50E+02 mM;
vii. L-Serine at a concentration between 3.50E−03 mM and 3.50E+02 mM;
viii. L-Arginine HCl at a concentration between 1.30E−02 mM and 1.30E+03 mM;
ix. L-Cystine at a concentration between 2.00E−03 mM and 2.00E+02 mM;
x. L-Histidine HCl—H₂O at a concentration between 3.50E−03 mM and 3.50E+02 mM;
xi. L-Isoleucine at a concentration between 8.20E−03 mM and 8.20E+02 mM;
xii. L-Leucine at a concentration between 8.50E−03 mM and 8.50E+02 mM;
xiii. L-Lysine hydrochloride at a concentration between 8.96E−03 mM and 8.96E+02 mM;
xiv. L-Methionine at a concentration between 2.21E−03 mM and 2.21E+02 mM;
xv. L-Phenylalanine at a concentration between 4.20E−03 mM and 4.20E+02 mM;
xvi. L-Threonine at a concentration between 8.50E−03 mM and 8.50E+02 mM;
xvii. L-Tryptophan at a concentration between 9.00E−04 mM and 9.00E+01 mM;
xviii. L-Tyrosine at a concentration between 4.09E−03 mM and 4.09E+02 mM;
xix. L-Valine at a concentration between 8.50E−03 mM and 8.50E+02 mM;
xx. Sodium Pyruvate at a concentration between 1.00E−02 mM and 1.00E+03 mM;
xxi. Arachidonic acid at a concentration between 3.28E−07 mM and 3.28E−02 mM;
xxii. Linoleic acid at a concentration between 3.78E−06 mM and 3.78E−01 mM;
xxiii. Linolenic acid at a concentration between 1.80E−06 mM and 1.80E−01 mM;
xxiv. Myristic acid at a concentration between 2.19E−06 mM and 2.19E−01 mM;
xxv. Oleic acid at a concentration between 1.77E−06 mM and 1.77E−01 mM;
xxvi. Palmitic acid at a concentration between 1.95E−06 mM and 1.95E−01 mM;
xxvii. Stearic acid at a concentration between 1.76E−06 mM and 1.76E−01 mM;
xxviii. Cholesterol at a concentration between 2.84E−05 mM and 2.84E+00 mM;
xxix. Tween-80 at a concentration between 8.40E−05 mM and 8.40E+00 mM;
xxx. Tocopherol acetate at a concentration between 7.40E−06 mM and 7.40E−01 mM;
xxxi. Pluronic F-68 at a concentration between 5.95E−04 mM and 5.95E+01 mM;
xxxii. Insulin at a concentration between 1.72E−05 mM and 1.72E+00 mM;
xxxiii. Transferrin at a concentration between 6.88E−07 mM and 6.88E−02 mM;
xxxiv. Sodium Selenite at a concentration between 3.87E−07 mM and 3.87E−02 mM;
xxxv. myo-Inositol at a concentration between 2.00E−03 mM and 2.00E+02 mM;
xxxvi. Glucose at a concentration between 2.22E−01 mM and 2.22E+04 mM;
xxxvii. Human Serum Albumin (HSA) at a concentration between 1.50E−05 mM and 1.50E+00 mM;
xxxviii. Biotin at a concentration between 8.10E−06 mM and 8.10E−01 mM;
xxxix. Folic Acid at a concentration between 2.40E−04 mM and 2.40E+01 mM;
xL. Vitamin B12 at a concentration between 1.00E−02 mM and 1.00E+03 mM;
xLi. Vitamin C at a concentration between 2.84E−03 mM and 2.84E+02 mM;
xLii. Vitamin E at a concentration between 2.40E−03 mM and 2.40E+02 mM;
xLiii. Zinc at a concentration between 1.50E−08 mM and 1.50E−03 mM;
xLiv. L-Ornithine at a concentration between 5.00E−04 mM and 5.00E+01 mM;
xLv. Creatine Anhydrous at a concentration between 1.00E−02 mM and 1.00E+03 mM;
xLvi. PolyVinyl Alcohol (PVA) at a concentration between 1.16E−01 and 1.16E+04 mM;
xLvii. Ethanolamine at a concentration between 5.00E−04 and 5.00E+01 mM;
xLviii. 2-mercaptoethanol (2-ME) at a concentration between 1.00E−03 mM and 1.00E+02 mM;
xLix. Androstenedione at a concentration between 1.00E−05 and 1.00E+00 mM;
L. Dextran at a concentration between 1.00E+01 and 1.00E+06 mg/L;
Li. L-Alanyl-L-Glutamine (Glutamax) at a concentration between 2.50E−02 mM and 2.50E+03 mM;
Lii. a polyamine supplement at a concentration between 0.01× and 1000× of a 1000× stock (polyamine supplement being Sigma-Aldrich Catalog #P8483 or equivalent); and
Liii. an antioxidant supplement at a concentration between 0.01× and 1000× of a 1000× (antioxidant supplement being Sigma-Aldrich Catalog #A1345 or equivalent); and
b. a second media formulation comprising the first media formulation and at least three of the following components:
Liv. Nicotinamide at a concentration between 2.50E−02 mM and 2.50E+03 mM;
Lv. IGF-1 (Insulin like Growth Factor 1) at a concentration between 2.60E−08 mM and 2.60E−03 mM;
Lvi. 4-1BBL [4-1BB Ligand or ligand for Cluster of Differentiation 137 (CD137)] at a concentration between 4.20E−09 mM and 4.20E−04 mM;
Lvii. Ashwagandha (botanical name: Withania Somnifera) plant extract at a concentration between 6.00E−01 mg/L and 6.00E+04 mg/L;
Lviii. Kumquat (botanical name: Fortunella Japonica) plant extract at a concentration between 1.40E−22 mg/L and 1.40E−02 mg/L;
Lix. Umckaloabo (botanical name: Pelargonium sidoides) plant extract at a concentration between 5.00E−01 mg/L and 5.00E+05 mg/L;
Lx. Echinacea (botanical name: Echinacea Purpurea) plant extract at a concentration between 1.01E−01 mg/L and 1.00E+05 mg/L;
Lxi. Red Ginseng (botanical name: Panax ginseng) plant extract at a concentration between 8.43E−02 mg/L and 8.43E+03 mg/L;
Lxii. 2,3-Butanediol at a concentration between 9.01E−02 mg/L and 9.01E+03 mg/L; and
Lxiii. Elderberry (botanical name: Sambucus nigra) plant extract at a concentration between 1.06E−01 mg/L and 1.06E+04 mg/L;
wherein the NK cells are cultured in the first media formulation prior to culture in the second media formulation.

2. A one-part media formulation for culturing Natural Killer (NK) cells comprising at least six of the following components:
i. Glycine at a concentration between 3.50E−03 mM and 3.50E+02 mM;
ii. L-Alanine at a concentration between 1.10E−02 mM and 1.10E+03 mM;
iii. L-Asparagine at a concentration between 1.50E−03 mM and 1.50E+02 mM;
iv. L-Aspartic acid at a concentration between 1.50E−03 mM and 1.50E+02 mM;
v. L-Glutamic Acid at a concentration between 1.50E−03 mM and 1.50E+02 mM;
vi. L-Proline at a concentration between 2.50E−03 mM and 2.50E+02 mM;
vii. L-Serine at a concentration between 3.50E−03 mM and 3.50E+02 mM;
viii. L-Arginine HCl at a concentration between 1.30E−02 mM and 1.30E+03 mM;
ix. L-Cystine at a concentration between 2.00E−03 mM and 2.00E+02 mM;
x. L-Histidine HCl—H₂O at a concentration between 3.50E−03 mM and 3.50E+02 mM;
xi. L-Isoleucine at a concentration between 8.20E−03 mM and 8.20E+02 mM;
xii. L-Leucine at a concentration between 8.50E−03 mM and 8.50E+02 mM;
xiii. L-Lysine hydrochloride at a concentration between 8.96E−03 mM and 8.96E+02 mM;
xiv. L-Methionine at a concentration between 2.21E−03 mM and 2.21E+02 mM;
xv. L-Phenylalanine at a concentration between 4.20E−03 mM and 4.20E+02 mM;
xvi. L-Threonine at a concentration between 8.50E−03 mM and 8.50E+02 mM;
xvii. L-Tryptophan at a concentration between 9.00E−04 mM and 9.00E+01 mM;
xviii. L-Tyrosine at a concentration between 4.09E−03 mM and 4.09E+02 mM;
xix. L-Valine at a concentration between 8.50E−03 mM and 8.50E+02 mM;
xx. Sodium Pyruvate at a concentration between 1.00E−02 mM and 1.00E+03 mM;
xxi. Arachidonic acid at a concentration between 3.28E−07 mM and 3.28E−02 mM;
xxii. Linoleic acid at a concentration between 3.78E−06 mM and 3.78E−01 mM;
xxiii. Linolenic acid at a concentration between 1.80E−06 mM and 1.80E−01 mM;
xxiv. Myristic acid at a concentration between 2.19E−06 mM and 2.19E−01 mM;
xxv. Oleic acid at a concentration between 1.77E−06 mM and 1.77E−01 mM;
xxvi. Palmitic acid at a concentration between 1.95E−06 mM and 1.95E−01 mM;
xxvii. Stearic acid at a concentration between 1.76E−06 mM and 1.76E−01 mM;
xxviii. Cholesterol at a concentration between 2.84E−05 mM and 2.84E+00 mM;
xxix. Tween-80 at a concentration between 8.40E−05 mM and 8.40E+00 mM;
xxx. Tocopherol acetate at a concentration between 7.40E−06 mM and 7.40E−01 mM;
xxxi. Pluronic F-68 at a concentration between 5.95E−04 mM and 5.95E+01 mM;
xxxii. Insulin at a concentration between 1.72E−05 mM and 1.72E+00 mM;
xxxiii. Transferrin at a concentration between 6.88E−07 mM and 6.88E−02 mM;
xxxiv. Sodium Selenite at a concentration between 3.87E−07 mM and 3.87E−02 mM;
xxxv. myo-Inositol at a concentration between 2.00E−03 mM and 2.00E+02 mM;
xxxvi. Glucose at a concentration between 2.22E−01 mM and 2.22E+04 mM;
xxxvii. Human Serum Albumin (HSA) at a concentration between 1.50E−05 mM and 1.50E+00 mM;
xxxviii. Biotin at a concentration between 8.10E−06 mM and 8.10E−01 mM;
xxxix. Folic Acid at a concentration between 2.40E−04 mM and 2.40E+01 mM;
xL. Vitamin B12 at a concentration between 1.00E−02 mM and 1.00E+03 mM;
xLi. Vitamin C at a concentration between 2.84E−03 mM and 2.84E+02 mM;
xLii. Vitamin E at a concentration between 2.40E−03 mM and 2.40E+02 mM;
xLiii. Zinc at a concentration between 1.50E−08 mM and 1.50E−03 mM;
xLiv. L-Ornithine at a concentration between 5.00E−04 mM and 5.00E+01 mM;
xLv. Creatine Anhydrous at a concentration between 1.00E−02 mM and 1.00E+03 mM;
xLvi. PolyVinyl Alcohol (PVA) at a concentration between 1.16E−01 and 1.16E+04 mM;
xLvii. Ethanolamine at a concentration between 5.00E−04 and 5.00E+01 mM;
xLviii. 2-mercaptoethanol (2-ME) at a concentration between 1.00E−03 mM and 1.00E+02 mM;
xLix. Androstenedione at a concentration between 1.00E−05 and 1.00E+00 mM;
L. Dextran at a concentration between 1.00E+01 and 1.00E+06 mg/L;
Li. L-Alanyl-L-Glutamine (Glutamax) at a concentration between 2.50E−02 mM and 2.50E+03 mM;
Lii. a polyamine supplement at a concentration between 0.01× and 1000× of a 1000× stock (polyamine supplement being Sigma-Aldrich Catalog #P8483 or equivalent); and
Liii. an antioxidant supplement at a concentration between 0.01× and 1000× of a 1000× (antioxidant supplement being Sigma-Aldrich Catalog #A1345 or equivalent).

3. A one-part media formulation culturing Natural Killer (NK) cells comprising at least nine of the following components:
i. Glycine at a concentration between 3.50E−03 mM and 3.50E+02 mM;
ii. L-Alanine at a concentration between 1.10E−02 mM and 1.10E+03 mM;
iii. L-Asparagine at a concentration between 1.50E−03 mM and 1.50E+02 mM;
iv. L-Aspartic acid at a concentration between 1.50E−03 mM and 1.50E+02 mM;
v. L-Glutamic Acid at a concentration between 1.50E−03 mM and 1.50E+02 mM;
vi. L-Proline at a concentration between 2.50E−03 mM and 2.50E+02 mM;
vii. L-Serine at a concentration between 3.50E−03 mM and 3.50E+02 mM;
viii. L-Arginine HCl at a concentration between 1.30E−02 mM and 1.30E+03 mM;
ix. L-Cystine at a concentration between 2.00E−03 mM and 2.00E+02 mM;
x. L-Histidine HCl—H₂O at a concentration between 3.50E−03 mM and 3.50E+02 mM;
xi. L-Isoleucine at a concentration between 8.20E−03 mM and 8.20E+02 mM;
xii. L-Leucine at a concentration between 8.50E−03 mM and 8.50E+02 mM;
xiii. L-Lysine hydrochloride at a concentration between 8.96E−03 mM and 8.96E+02 mM;
xiv. L-Methionine at a concentration between 2.21E−03 mM and 2.21E+02 mM;
xv. L-Phenylalanine at a concentration between 4.20E−03 mM and 4.20E+02 mM;
xvi. L-Threonine at a concentration between 8.50E−03 mM and 8.50E+02 mM;
xvii. L-Tryptophan at a concentration between 9.00E−04 mM and 9.00E+01 mM;
xviii. L-Tyrosine at a concentration between 4.09E−03 mM and 4.09E+02 mM;
xix. L-Valine at a concentration between 8.50E−03 mM and 8.50E+02 mM;
xx. Sodium Pyruvate at a concentration between 1.00E−02 mM and 1.00E+03 mM;
xxi. Arachidonic acid at a concentration between 3.28E−07 mM and 3.28E−02 mM;
xxii. Linoleic acid at a concentration between 3.78E−06 mM and 3.78E−01 mM;
xxiii. Linolenic acid at a concentration between 1.80E−06 mM and 1.80E−01 mM;
xxiv. Myristic acid at a concentration between 2.19E−06 mM and 2.19E−01 mM;
xxv. Oleic acid at a concentration between 1.77E−06 mM and 1.77E−01 mM;
xxvi. Palmitic acid at a concentration between 1.95E−06 mM and 1.95E−01 mM;
xxvii. Stearic acid at a concentration between 1.76E−06 mM and 1.76E−01 mM;
xxviii. Cholesterol at a concentration between 2.84E−05 mM and 2.84E+00 mM;
xxix. Tween-80 at a concentration between 8.40E−05 mM and 8.40E+00 mM;
xxx. Tocopherol acetate at a concentration between 7.40E−06 mM and 7.40E−01 mM;
xxxi. Pluronic F-68 at a concentration between 5.95E−04 mM and 5.95E+01 mM;
xxxii. Insulin at a concentration between 1.72E−05 mM and 1.72E+00 mM;
xxxiii. Transferrin at a concentration between 6.88E−07 mM and 6.88E−02 mM;
xxxiv. Sodium Selenite at a concentration between 3.87E−07 mM and 3.87E−02 mM;
xxxv. myo-Inositol at a concentration between 2.00E−03 mM and 2.00E+02 mM;
xxxvi. Glucose at a concentration between 2.22E−01 mM and 2.22E+04 mM;
xxxvii. Human Serum Albumin (HSA) at a concentration between 1.50E−05 mM and 1.50E+00 mM;
xxxviii. Biotin at a concentration between 8.10E−06 mM and 8.10E−01 mM;
xxxix. Folic Acid at a concentration between 2.40E−04 mM and 2.40E+01 mM;
xL. Vitamin B12 at a concentration between 1.00E−02 mM and 1.00E+03 mM;
xLi. Vitamin C at a concentration between 2.84E−03 mM and 2.84E+02 mM;
xLii. Vitamin E at a concentration between 2.40E−03 mM and 2.40E+02 mM;
xLiii. Zinc at a concentration between 1.50E−08 mM and 1.50E−03 mM;
xLiv. L-Ornithine at a concentration between 5.00E−04 mM and 5.00E+01 mM;
xLv. Creatine Anhydrous at a concentration between 1.00E−02 mM and 1.00E+03 mM;
xLvi. PolyVinyl Alcohol (PVA) at a concentration between 1.16E−01 and 1.16E+04 mM;
xLvii. Ethanolamine at a concentration between 5.00E−04 and 5.00E+01 mM;
xLviii. 2-mercaptoethanol (2-ME) at a concentration between 1.00E−03 mM and 1.00E+02 mM;
xLix. Androstenedione at a concentration between 1.00E−05 and 1.00E+00 mM;
L. Dextran at a concentration between 1.00E+01 and 1.00E+06 mg/L;
Li. L-Alanyl-L-Glutamine (Glutamax) at a concentration between 2.50E−02 mM and 2.50E+03 mM;
Lii. a polyamine supplement at a concentration between 0.01× and 1000× of a 1000× stock (polyamine supplement being Sigma-Aldrich Catalog #P8483 or equivalent);
Liii. an antioxidant supplement at a concentration between 0.01× and 1000× of a 1000× (antioxidant supplement being Sigma-Aldrich Catalog #A1345 or equivalent);
Liv. Nicotinamide at a concentration between 2.50E−02 mM and 2.50E+03 mM;
Lv. IGF-1 (Insulin like Growth Factor 1) at a concentration between 2.60E−08 mM and 2.60E−03 mM;
Lvi. 4-1BBL [4-1BB Ligand or ligand for Cluster of Differentiation 137 (CD137)] at a concentration between 4.20E−09 mM and 4.20E−04 mM;
Lvii. Ashwagandha (botanical name: Withania Somnifera) plant extract at a concentration between 6.00E−01 mg/L and 6.00E+04 mg/L;
Lviii. Kumquat (botanical name: Fortunella Japonica) plant extract at a concentration between 1.40E−22 mg/L and 1.40E−02 mg/L;
Lix. Umckaloabo (botanical name: Pelargonium sidoides) plant extract at a concentration between 5.00E−01 mg/L and 5.00E+05 mg/L;
Lx. Echinacea (botanical name: Echinacea Purpurea) plant extract at a concentration between 1.01E−01 mg/L and 1.00E+05 mg/L;
Lxi. Red Ginseng (botanical name: Panax ginseng) plant extract at a concentration between 8.43E−02 mg/L and 8.43E+03 mg/L;
Lxii. 2,3-Butanediol at a concentration between 9.01E−02 mg/L and 9.01E+03 mg/L; and
Lxiii. Elderberry (botanical name: Sambucus nigra) plant extract at a concentration between 1.06E−01 mg/L and 1.06E+04 mg/L

4. The formulation of any one of claims 1-3, wherein the formulation is added to a base cell culture media as a supplement.

5. A method for culturing Natural Killer (NK) cells or precursor cells thereof comprising culturing the NK cells or precursor cells thereof in a cell culture media formulation comprising:
a. a first media formulation comprising at least six of the following components:
i. Glycine at a concentration between 3.50E−03 mM and 3.50E+02 mM;
ii. L-Alanine at a concentration between 1.10E−02 mM and 1.10E+03 mM;
iii. L-Asparagine at a concentration between 1.50E−03 mM and 1.50E+02 mM;
iv. L-Aspartic acid at a concentration between 1.50E−03 mM and 1.50E+02 mM;
v. L-Glutamic Acid at a concentration between 1.50E−03 mM and 1.50E+02 mM;
vi. L-Proline at a concentration between 2.50E−03 mM and 2.50E+02 mM;
vii. L-Serine at a concentration between 3.50E−03 mM and 3.50E+02 mM;
viii. L-Arginine HCl at a concentration between 1.30E−02 mM and 1.30E+03 mM;
ix. L-Cystine at a concentration between 2.00E−03 mM and 2.00E+02 mM;
x. L-Histidine HCl—H₂O at a concentration between 3.50E−03 mM and 3.50E+02 mM;
xi. L-Isoleucine at a concentration between 8.20E−03 mM and 8.20E+02 mM;
xii. L-Leucine at a concentration between 8.50E−03 mM and 8.50E+02 mM;
xiii. L-Lysine hydrochloride at a concentration between 8.96E−03 mM and 8.96E+02 mM;
xiv. L-Methionine at a concentration between 2.21E−03 mM and 2.21E+02 mM;
xv. L-Phenylalanine at a concentration between 4.20E−03 mM and 4.20E+02 mM;
xvi. L-Threonine at a concentration between 8.50E−03 mM and 8.50E+02 mM;
xvii. L-Tryptophan at a concentration between 9.00E−04 mM and 9.00E+01 mM;
xviii. L-Tyrosine at a concentration between 4.09E−03 mM and 4.09E+02 mM;
xix. L-Valine at a concentration between 8.50E−03 mM and 8.50E+02 mM;
xx. Sodium Pyruvate at a concentration between 1.00E−02 mM and 1.00E+03 mM;
xxi. Arachidonic acid at a concentration between 3.28E−07 mM and 3.28E−02 mM;
xxii. Linoleic acid at a concentration between 3.78E−06 mM and 3.78E−01 mM;
xxiii. Linolenic acid at a concentration between 1.80E−06 mM and 1.80E−01 mM;
xxiv. Myristic acid at a concentration between 2.19E−06 mM and 2.19E−01 mM;
xxv. Oleic acid at a concentration between 1.77E−06 mM and 1.77E−01 mM;
xxvi. Palmitic acid at a concentration between 1.95E−06 mM and 1.95E−01 mM;
xxvii. Stearic acid at a concentration between 1.76E−06 mM and 1.76E−01 mM;
xxviii. Cholesterol at a concentration between 2.84E−05 mM and 2.84E+00 mM;
xxix. Tween-80 at a concentration between 8.40E−05 mM and 8.40E+00 mM;
xxx. Tocopherol acetate at a concentration between 7.40E−06 mM and 7.40E−01 mM;
xxxi. Pluronic F-68 at a concentration between 5.95E−04 mM and 5.95E+01 mM;
xxxii. Insulin at a concentration between 1.72E−05 mM and 1.72E+00 mM;
xxxiii. Transferrin at a concentration between 6.88E−07 mM and 6.88E−02 mM;
xxxiv. Sodium Selenite at a concentration between 3.87E−07 mM and 3.87E−02 mM;
xxxv. myo-Inositol at a concentration between 2.00E−03 mM and 2.00E+02 mM;
xxxvi. Glucose at a concentration between 2.22E−01 mM and 2.22E+04 mM;
xxxvii. Human Serum Albumin (HSA) at a concentration between 1.50E−05 mM and 1.50E+00 mM;
xxxviii. Biotin at a concentration between 8.10E−06 mM and 8.10E−01 mM;
xxxix. Folic Acid at a concentration between 2.40E−04 mM and 2.40E+01 mM;
xL. Vitamin B12 at a concentration between 1.00E−02 mM and 1.00E+03 mM;
xLi. Vitamin C at a concentration between 2.84E−03 mM and 2.84E+02 mM;
xLii. Vitamin E at a concentration between 2.40E−03 mM and 2.40E+02 mM;
xLiii. Zinc at a concentration between 1.50E−08 mM and 1.50E−03 mM;
xLiv. L-Ornithine at a concentration between 5.00E−04 mM and 5.00E+01 mM;
xLv. Creatine Anhydrous at a concentration between 1.00E−02 mM and 1.00E+03 mM;
xLvi. PolyVinyl Alcohol (PVA) at a concentration between 1.16E−01 and 1.16E+04 mM;
xLvii. Ethanolamine at a concentration between 5.00E−04 and 5.00E+01 mM;
xLviii. 2-mercaptoethanol (2-ME) at a concentration between 1.00E−03 mM and 1.00E+02 mM;
xLix. Androstenedione at a concentration between 1.00E−05 and 1.00E+00 mM;
L. Dextran at a concentration between 1.00E+01 and 1.00E+06 mg/L;
Li. L-Alanyl-L-Glutamine (Glutamax) at a concentration between 2.50E−02 mM and 2.50E+03 mM;
Lii. a polyamine supplement at a concentration between 0.01× and 1000× of a 1000× stock (polyamine supplement being Sigma-Aldrich Catalog #P8483 or equivalent); and
Liii. an antioxidant supplement at a concentration between 0.01× and 1000× of a 1000× (antioxidant supplement being Sigma-Aldrich Catalog #A1345 or equivalent); and
b. a second media formulation comprising the first media formulation and at least three of the following components:
Liv. Nicotinamide at a concentration between 2.50E−02 mM and 2.50E+03 mM;
Lv. IGF-1 (Insulin like Growth Factor 1) at a concentration between 2.60E−08 mM and 2.60E−03 mM;
Lvi. 4-1BBL [4-1BB Ligand or ligand for Cluster of Differentiation 137 (CD137)] at a concentration between 4.20E−09 mM and 4.20E−04 mM;
Lvii. Ashwagandha (botanical name: Withania Somnifera) plant extract at a concentration between 6.00E−01 mg/L and 6.00E+04 mg/L;
Lviii. Kumquat (botanical name: Fortunella Japonica) plant extract at a concentration between 1.40E−22 mg/L and 1.40E−02 mg/L;
Lix. Umckaloabo (botanical name: Pelargonium sidoides) plant extract at a concentration between 5.00E−01 mg/L and 5.00E+05 mg/L;
Lx. Echinacea (botanical name: Echinacea Purpurea) plant extract at a concentration between 1.01E−01 mg/L and 1.00E+05 mg/L;
Lxi. Red Ginseng (botanical name: Panax ginseng) plant extract at a concentration between 8.43E−02 mg/L and 8.43E+03 mg/L;
Lxii. 2,3-Butanediol at a concentration between 9.01E−02 mg/L and 9.01E+03 mg/L; and
Lxiii. Elderberry (botanical name: Sambucus nigra) plant extract at a concentration between 1.06E−01 mg/L and 1.06E+04 mg/L.

6. The method of claim 5, wherein the NK cells or precursor cells thereof are cultured in the first media formulation.

7. The method of claim 5, wherein the NK cells or precursor cells thereof are cultured in the second media formulation.

8. The method of claim 5, wherein the NK cells or precursor cells thereof are cultured in the first media formulation followed by the second formulation.

9. The method of claim 5, wherein the NK cells or precursor cells thereof are cultured ex vivo.

10. The method of claim 5, wherein the NK cells or precursor cells thereof are cultured in vitro.

11. The method of claim 5, wherein cytotoxic activity of the NK cells against target cells is enhanced.

12. The method of claim 11, wherein the target cells are cancer cells.

13. The method of claim 5, wherein activation of the NK cells is enhanced.

14. The method of claim 5, wherein dysfunction of the NK cells is reduced.

15. The method of claim 14, wherein the dysfunction is exhaustion.

16. The method of claim 5, wherein metabolic function of the NK cells is enhanced.

17. The method of claim 5, wherein high viability and cell-proliferation of NK cells is achieved.

18. The method of claim 5, wherein use of serum is eliminated or reduced.

19. The method of claim 5, wherein use of cytokine supplements is eliminated or reduced.

20. The method of claim 5, wherein the use of Antigen Presenting Cells (APCs) or feeder cells is eliminated or reduced.

21. The method of claim 5, wherein the culture of NK precursor cells induces their differentiation.

22. The method of claim 5, wherein the culture of NK cells or precursor cells modulates the distribution and relative populations of NK cell subsets.

23. The method of claim 22, wherein the NK cell subsets are memory NK cells.

24. The method of claim 22, wherein the NK cell subsets are defined by relative expression of activating and inhibitory receptors.

25. A method for cryopreservation of Natural Killer (NK) cells, wherein freezing the NK cells is accomplished in a cryopreservation medium comprising:
a. a first media formulation comprising at least six of the following components:
i. Glycine at a concentration between 3.50E−03 mM and 3.50E+02 mM;
ii. L-Alanine at a concentration between 1.10E−02 mM and 1.10E+03 mM;
iii. L-Asparagine at a concentration between 1.50E−03 mM and 1.50E+02 mM;
iv. L-Aspartic acid at a concentration between 1.50E−03 mM and 1.50E+02 mM;
v. L-Glutamic Acid at a concentration between 1.50E−03 mM and 1.50E+02 mM;
vi. L-Proline at a concentration between 2.50E−03 mM and 2.50E+02 mM;
vii. L-Serine at a concentration between 3.50E−03 mM and 3.50E+02 mM;
viii. L-Arginine HCl at a concentration between 1.30E−02 mM and 1.30E+03 mM;
ix. L-Cystine at a concentration between 2.00E−03 mM and 2.00E+02 mM;
x. L-Histidine HCl—H₂O at a concentration between 3.50E−03 mM and 3.50E+02 mM;
xi. L-Isoleucine at a concentration between 8.20E−03 mM and 8.20E+02 mM;
xii. L-Leucine at a concentration between 8.50E−03 mM and 8.50E+02 mM;
xiii. L-Lysine hydrochloride at a concentration between 8.96E−03 mM and 8.96E+02 mM;
xiv. L-Methionine at a concentration between 2.21E−03 mM and 2.21E+02 mM;
xv. L-Phenylalanine at a concentration between 4.20E−03 mM and 4.20E+02 mM;
xvi. L-Threonine at a concentration between 8.50E−03 mM and 8.50E+02 mM;
xvii. L-Tryptophan at a concentration between 9.00E−04 mM and 9.00E+01 mM;
xviii. L-Tyrosine at a concentration between 4.09E−03 mM and 4.09E+02 mM;
xix. L-Valine at a concentration between 8.50E−03 mM and 8.50E+02 mM;
xx. Sodium Pyruvate at a concentration between 1.00E−02 mM and 1.00E+03 mM;
xxi. Arachidonic acid at a concentration between 3.28E−07 mM and 3.28E−02 mM;
xxii. Linoleic acid at a concentration between 3.78E−06 mM and 3.78E−01 mM;
xxiii. Linolenic acid at a concentration between 1.80E−06 mM and 1.80E−01 mM;
xxiv. Myristic acid at a concentration between 2.19E−06 mM and 2.19E−01 mM;
xxv. Oleic acid at a concentration between 1.77E−06 mM and 1.77E−01 mM;
xxvi. Palmitic acid at a concentration between 1.95E−06 mM and 1.95E−01 mM;
xxvii. Stearic acid at a concentration between 1.76E−06 mM and 1.76E−01 mM;
xxviii. Cholesterol at a concentration between 2.84E−05 mM and 2.84E+00 mM;
xxix. Tween-80 at a concentration between 8.40E−05 mM and 8.40E+00 mM;
xxx. Tocopherol acetate at a concentration between 7.40E−06 mM and 7.40E−01 mM;
xxxi. Pluronic F-68 at a concentration between 5.95E−04 mM and 5.95E+01 mM;
xxxii. Insulin at a concentration between 1.72E−05 mM and 1.72E+00 mM;
xxxiii. Transferrin at a concentration between 6.88E−07 mM and 6.88E−02 mM;
xxxiv. Sodium Selenite at a concentration between 3.87E−07 mM and 3.87E−02 mM;
xxxv. myo-Inositol at a concentration between 2.00E−03 mM and 2.00E+02 mM;
xxxvi. Glucose at a concentration between 2.22E−01 mM and 2.22E+04 mM;
xxxvii. Human Serum Albumin (HSA) at a concentration between 1.50E−05 mM and 1.50E+00 mM;
xxxviii. Biotin at a concentration between 8.10E−06 mM and 8.10E−01 mM;
xxxix. Folic Acid at a concentration between 2.40E−04 mM and 2.40E+01 mM;
xL. Vitamin B12 at a concentration between 1.00E−02 mM and 1.00E+03 mM;
xLi. Vitamin C at a concentration between 2.84E−03 mM and 2.84E+02 mM;
xLii. Vitamin E at a concentration between 2.40E−03 mM and 2.40E+02 mM;
xLiii. Zinc at a concentration between 1.50E−08 mM and 1.50E−03 mM;
xLiv. L-Ornithine at a concentration between 5.00E−04 mM and 5.00E+01 mM;
xLv. Creatine Anhydrous at a concentration between 1.00E−02 mM and 1.00E+03 mM;
xLvi. PolyVinyl Alcohol (PVA) at a concentration between 1.16E−01 and 1.16E+04 mM;
xLvii. Ethanolamine at a concentration between 5.00E−04 and 5.00E+01 mM;
xLviii. 2-mercaptoethanol (2-ME) at a concentration between 1.00E−03 mM and 1.00E+02 mM;
xLix. Androstenedione at a concentration between 1.00E−05 and 1.00E+00 mM;
L. Dextran at a concentration between 1.00E+01 and 1.00E+06 mg/L;
Li. L-Alanyl-L-Glutamine (Glutamax) at a concentration between 2.50E−02 mM and 2.50E+03 mM;
Lii. a polyamine supplement at a concentration between 0.01× and 1000× of a 1000× stock (polyamine supplement being Sigma-Aldrich Catalog #P8483 or equivalent); and
Liii. an antioxidant supplement at a concentration between 0.01× and 1000× of a 1000× (antioxidant supplement being Sigma-Aldrich Catalog #A1345 or equivalent); and
b. a second media formulation comprising the first media formulation and at least three of the following components:
Liv. Nicotinamide at a concentration between 2.50E−02 mM and 2.50E+03 mM;
Lv. IGF-1 (Insulin like Growth Factor 1) at a concentration between 2.60E−08 mM and 2.60E−03 mM;
Lvi. 4-1BBL [4-1BB Ligand or ligand for Cluster of Differentiation 137 (CD137)] at a concentration between 4.20E−09 mM and 4.20E−04 mM;
Lvii. Ashwagandha (botanical name: Withania Somnifera) plant extract at a concentration between 6.00E−01 mg/L and 6.00E+04 mg/L;
Lviii. Kumquat (botanical name: Fortunella Japonica) plant extract at a concentration between 1.40E−22 mg/L and 1.40E−02 mg/L;
Lix. Umckaloabo (botanical name: Pelargonium sidoides) plant extract at a concentration between 5.00E−01 mg/L and 5.00E+05 mg/L;
Lx. Echinacea (botanical name: Echinacea Purpurea) plant extract at a concentration between 1.01E−01 mg/L and 1.00E+05 mg/L;
Lxi. Red Ginseng (botanical name: Panax ginseng) plant extract at a concentration between 8.43E−02 mg/L and 8.43E+03 mg/L;
Lxii. 2,3-Butanediol at a concentration between 9.01E−02 mg/L and 9.01E+03 mg/L; and
Lxiii. Elderberry (botanical name: Sambucus nigra) plant extract at a concentration between 1.06E−01 mg/L and 1.06E+04 mg/L.

26. The method of claim 25, wherein the cryopreservation medium comprises the first media formulation.

27. The method of claim 25, wherein the cryopreservation medium comprises the second media formulation.

28. A method for transduction of Natural Killer (NK) immune cells comprising
a. a first cell culture step comprising culturing the NK cells in a cell culture medium comprising:
a first media formulation comprising at least six of the following components:
i. Glycine at a concentration between 3.50E−03 mM and 3.50E+02 mM;
ii. L-Alanine at a concentration between 1.10E−02 mM and 1.10E+03 mM;
iii. L-Asparagine at a concentration between 1.50E−03 mM and 1.50E+02 mM;
iv. L-Aspartic acid at a concentration between 1.50E−03 mM and 1.50E+02 mM;
v. L-Glutamic Acid at a concentration between 1.50E−03 mM and 1.50E+02 mM;
vi. L-Proline at a concentration between 2.50E−03 mM and 2.50E+02 mM;
vii. L-Serine at a concentration between 3.50E−03 mM and 3.50E+02 mM;
viii. L-Arginine HCl at a concentration between 1.30E−02 mM and 1.30E+03 mM;
ix. L-Cystine at a concentration between 2.00E−03 mM and 2.00E+02 mM;
x. L-Histidine HCl—H₂O at a concentration between 3.50E−03 mM and 3.50E+02 mM;
xi. L-Isoleucine at a concentration between 8.20E−03 mM and 8.20E+02 mM;
xii. L-Leucine at a concentration between 8.50E−03 mM and 8.50E+02 mM;
xiii. L-Lysine hydrochloride at a concentration between 8.96E−03 mM and 8.96E+02 mM;
xiv. L-Methionine at a concentration between 2.21E−03 mM and 2.21E+02 mM;
xv. L-Phenylalanine at a concentration between 4.20E−03 mM and 4.20E+02 mM;
xvi. L-Threonine at a concentration between 8.50E−03 mM and 8.50E+02 mM;
xvii. L-Tryptophan at a concentration between 9.00E−04 mM and 9.00E+01 mM;
xviii. L-Tyrosine at a concentration between 4.09E−03 mM and 4.09E+02 mM;
xix. L-Valine at a concentration between 8.50E−03 mM and 8.50E+02 mM;
xx. Sodium Pyruvate at a concentration between 1.00E−02 mM and 1.00E+03 mM;
xxi. Arachidonic acid at a concentration between 3.28E−07 mM and 3.28E−02 mM;
xxii. Linoleic acid at a concentration between 3.78E−06 mM and 3.78E−01 mM;
xxiii. Linolenic acid at a concentration between 1.80E−06 mM and 1.80E−01 mM;
xxiv. Myristic acid at a concentration between 2.19E−06 mM and 2.19E−01 mM;
xxv. Oleic acid at a concentration between 1.77E−06 mM and 1.77E−01 mM;
xxvi. Palmitic acid at a concentration between 1.95E−06 mM and 1.95E−01 mM;
xxvii. Stearic acid at a concentration between 1.76E−06 mM and 1.76E−01 mM;
xxviii. Cholesterol at a concentration between 2.84E−05 mM and 2.84E+00 mM;
xxix. Tween-80 at a concentration between 8.40E−05 mM and 8.40E+00 mM;
xxx. Tocopherol acetate at a concentration between 7.40E−06 mM and 7.40E−01 mM;
xxxi. Pluronic F-68 at a concentration between 5.95E−04 mM and 5.95E+01 mM;
xxxii. Insulin at a concentration between 1.72E−05 mM and 1.72E+00 mM;
xxxiii. Transferrin at a concentration between 6.88E−07 mM and 6.88E−02 mM;
xxxiv. Sodium Selenite at a concentration between 3.87E−07 mM and 3.87E−02 mM;
xxxv. myo-Inositol at a concentration between 2.00E−03 mM and 2.00E+02 mM;
xxxvi. Glucose at a concentration between 2.22E−01 mM and 2.22E+04 mM;
xxxvii. Human Serum Albumin (HSA) at a concentration between 1.50E−05 mM and 1.50E+00 mM;
xxxviii. Biotin at a concentration between 8.10E−06 mM and 8.10E−01 mM;
xxxix. Folic Acid at a concentration between 2.40E−04 mM and 2.40E+01 mM;
xL. Vitamin B12 at a concentration between 1.00E−02 mM and 1.00E+03 mM;
xLi. Vitamin C at a concentration between 2.84E−03 mM and 2.84E+02 mM;
xLii. Vitamin E at a concentration between 2.40E−03 mM and 2.40E+02 mM;
xLiii. Zinc at a concentration between 1.50E−08 mM and 1.50E−03 mM;
xLiv. L-Ornithine at a concentration between 5.00E−04 mM and 5.00E+01 mM;
xLv. Creatine Anhydrous at a concentration between 1.00E−02 mM and 1.00E+03 mM;
xLvi. PolyVinyl Alcohol (PVA) at a concentration between 1.16E−01 and 1.16E+04 mM;
xLvii. Ethanolamine at a concentration between 5.00E−04 and 5.00E+01 mM;
xLviii. 2-mercaptoethanol (2-ME) at a concentration between 1.00E−03 mM and 1.00E+02 mM;
xLix. Androstenedione at a concentration between 1.00E−05 and 1.00E+00 mM;
L. Dextran at a concentration between 1.00E+01 and 1.00E+06 mg/L;
Li. L-Alanyl-L-Glutamine (Glutamax) at a concentration between 2.50E−02 mM and 2.50E+03 mM;
Lii. a polyamine supplement at a concentration between 0.01× and 1000× of a 1000× stock (polyamine supplement being Sigma-Aldrich Catalog #P8483 or equivalent); and
Liii. an antioxidant supplement at a concentration between 0.01× and 1000× of a 1000× (antioxidant supplement being Sigma-Aldrich Catalog #A1345 or equivalent); and
a second media formulation comprising the first media formulation and at least three of the following components:
Liv. Nicotinamide at a concentration between 2.50E−02 mM and 2.50E+03 mM;
Lv. IGF-1 (Insulin like Growth Factor 1) at a concentration between 2.60E−08 mM and 2.60E−03 mM;
Lvi. 4-1BBL [4-1BB Ligand or ligand for Cluster of Differentiation 137 (CD137)] at a concentration between 4.20E−09 mM and 4.20E−04 mM;
Lvii. Ashwagandha (botanical name: Withania Somnifera) plant extract at a concentration between 6.00E−01 mg/L and 6.00E+04 mg/L;
Lviii. Kumquat (botanical name: Fortunella Japonica) plant extract at a concentration between 1.40E−22 mg/L and 1.40E−02 mg/L;
Lix. Umckaloabo (botanical name: Pelargonium sidoides) plant extract at a concentration between 5.00E−01 mg/L and 5.00E+05 mg/L;
Lx. Echinacea (botanical name: Echinacea Purpurea) plant extract at a concentration between 1.01E−01 mg/L and 1.00E+05 mg/L;
Lxi. Red Ginseng (botanical name: Panax ginseng) plant extract at a concentration between 8.43E−02 mg/L and 8.43E+03 mg/L;
Lxii. 2,3-Butanediol at a concentration between 9.01E−02 mg/L and 9.01E+03 mg/L; and
Lxiii. Elderberry (botanical name: Sambucus nigra) plant extract at a concentration between 1.06E−01 mg/L and 1.06E+04 mg/L;
b. contacting the NK cells with a viral vector; and
c. a second cell culture step comprising culturing the transduced NK cells in a cell culture medium comprising the cell culture medium formulation of step a.

29. The method of claim 28, wherein the first and second cell cultures steps are carried out in the first cell culture formulation.

30. The method of claim 28, wherein the first and second cell culture steps are carried out in the second cell culture formulation.

31. The method of claim 28, wherein the first cell culture step is carried out in the first cell culture formulation and the second cell culture step is carried out in the second cell culture formulation.

32. The method of claim 28, wherein the vector encodes a chimeric antigen receptor (CAR).
